SEQUENCE LISTING (79 sequences)

SEQ ID NO: 1 - protein, 349 aa, Homo sapiens
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA

SEQ ID NO: 2 - protein, 349 aa, synthetic construct
  REGION 1..349: note = N-glycosylation knocked-out NTCP (NTCP_WT_KOG)
MEAHTASAPFTFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA

SEQ ID NO: 3 - protein, 327 aa, synthetic construct
  REGION 1..327: note = NTCP consensus-mutant construct used for animal immunization (NTCP_CO)
MAHTASAPFTFTLPPNFGKRPTDLALSVILVVMLFIIMLSLGCTMEFSKIKAHLWKPKGLAIALVAQYGIMPLTAFVLGKVFRLNNIEALAILICGCSPGGNLSNIFSLALKGDMNLSIVMTTCSTFLALGMMPLLLYIYSKGIYDGDLKDKVPYKGIVISLVLVLIPCTIGIILNSKRPQYVRYVIKGGMIILLLSSVAITVLSAINVGKSIMFVMTPHLLATSSLMPFIGFLLGYILSALFRLNGRCRRTVSMETGCQNVQLCSTILNVAFPPEVIGPLFFFPLLYMIFQLGEGLLLIAIFRCYEKIKPPKDKTKMIYTAATTEE

SEQ ID NO: 4 - protein, 327 aa, synthetic construct
  REGION 1..327: note = NTCP consensus-mutant construct used for cryo-electron microscopy structure determination (NTCP_EM)
MAHTASAPFTFTLPPNFGKRPTDLALSVILVVMLFIIMLSLGCTMEFSKIKAHLWKPKGLAIALVAQYGIMPLTAFVLGKVFRLNNIEALAILICGCSPGGNLSNIFSLAMKGDMNLSIVMTTCSTFLALGMMPLLLYIYSRGIYDGDLKDKVPYKGIVISLVLVLIPCTIGIVLKSKRPQYMRYVIKGGMIIILLCSVAVTVLSAINVGKSIMFAMTPHLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEE

SEQ ID NO: 5 - protein, 126 aa, synthetic construct
  REGION 1..126: note = Nb NTCP_87
QVQLVESGGGLVQAGGSLRLSCAVSGRTTANYNMGWFRQAPGKEREFVAGIKWSSGSTYVADSAKGRFTISRDNAKNSVYLQMDSLKPEDTALYYCAANYYGVSWFLISPSSYDYWGQGTQVTVSS

SEQ ID NO: 6 - protein, 126 aa, synthetic construct
  REGION 1..126: note = Nb NTCP_88
QVQLVESGGGLVQAGGSLRLSCAASGRTSDNYNMGWFRQAPGKEREFVAGIKWSSGSTYHADSAKGRFTISRDNAKNTVYLQMNSLKPEDTALYYCAANYYGVSWFLISPSSYDYWGQGTQVTVSS

SEQ ID NO: 7 - protein, 125 aa, synthetic construct
  REGION 1..125: note = Nb NTCP_91
QVQLVESGGGLVEAGGSLRLSCAASTNLRSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 8 - protein, 126 aa, synthetic construct
  REGION 1..126: note = Nb NTCP_79
QVQLVESGGGLVQVGDSLRLSCAASGRTFSSYVMGWFRQAPGKDREFVATVSRRGINTYYADSVKGRFTISRDNAKNTVYLQMNNLKPEDAAAYYCAADLNPKYPVTILTSDYDYWGQGTQVTVSS

SEQ ID NO: 9 - protein, 126 aa, synthetic construct
  REGION 1..126: note = Nb NTCP_80
QVQLVESGGGLVQVGDSLRLSCAASGRTFSSYVMGWFRQAPGKEREFVATISRRGINTYYADSVKGRFTISRDNAKNTVYLQMNNLKPEDAAGYYCAADLNPKYPVTILTSDYDYWGQGTQVTVSS

SEQ ID NO: 10 - protein, 126 aa, synthetic construct
  REGION 1..126: note = Nb NTCP_81
QVQLVESGGGLVQVGDSLRLSCAASGRTFSDYVMGWFRQAPGKEREFVATVSRRGINTYYADSVKDRFTISRDNAKNTVYLQMNNLKPEDAAAYYCAADFNPKYPLTIKTSDYDYWGQGTQVTVSS

SEQ ID NO: 11 - protein, 121 aa, synthetic construct
  REGION 1..121: note = Nb NTCP_83
QVQLVESGGGLVQPGGSLRLSCAASGSIFSLNTLGWYRQAPGKQRELVATITHGGSTKYADSVLGRFTISRDNAKNTVYLQMYSLKPEDTAVYYCHADGGSSWFEDWPDYWGQGTQVTVSS

SEQ ID NO: 12 - protein, 121 aa, synthetic construct
  REGION 1..121: note = Nb NTCP_82
QVQLVESGGGLVQPGGSLRLSCAASGSIFSINTLGWYRQAPGKQRELVATITHGGSTKYADSVSGRFTISRDNAKNTVYLQMNSLKPEDTAVYYCNADGGSSWFEEWPDYWGQGTQVTVSS

SEQ ID NO: 13 - protein, 120 aa, synthetic construct
  REGION 1..120: note = Nb NTCP_84
QVQLVESGGGLVQAGGSLRLSCAASGTFFSINAMGWYRQTPGQQRELVAAITSGGSTNYADSVKGRFTISRDNAKNTVYLQMNSLKPEDTAVYYCHADGGSSWFESWDYWGQGTQVTVSS

SEQ ID NO: 14 - protein, 119 aa, synthetic construct
  REGION 1..119: note = Nb NTCP_53
QVQLVESGGGLVQAGGSLRLSCTASGRIFSWLDMAWYRQAPGKQRELVASITSVGTANYADSVKGRFTISRDNAKNTVYLQMNSLKPEDTAVYSCNAKYSSWSGERSDWGQGTQVTVSS

SEQ ID NO: 15 - protein, 120 aa, synthetic construct
  REGION 1..120: note = Nb NTCP_74
QVQLVESGGGLVQSGGSLRLSCAASTNMVSINSMGWYRQTPGKQRELVAGATTGGSTDYADSVKGRFTISRDNAMNTVYLQMNGLKPEDTATYYCNAIGVSRWGARYDYWGQGTQVTVSS

SEQ ID NO: 16 - protein, 120 aa, synthetic construct
  REGION 1..120: note = Nb NTCP_75
QVQLVESGGGLVQSGGSLTLSCAASVSSVSINSMAWYRQAPGKERELVAAVTSGGRIDYSDSVKGRFTISRDNAKNTLYLQMNGLKPEDTAAYYCNLMGTSRWGARYDYWGQGTQVTVSS

SEQ ID NO: 17 - protein, 120 aa, synthetic construct
  REGION 1..120: note = Nb NTCP_76
QVQLVESGGGLVQTGGSLRLSCAASANAVRINAMAWYRQAPGKQRELVAVVTSGGNTDYGDSVKGRFTISRDNAKKSLYLQMNSLKPEDTAVYYCNVDGTSVWGAKYDYWGQGTQVTVSS

SEQ ID NO: 18 - protein, 119 aa, synthetic construct
  REGION 1..119: note = Nb NTCP_77
QVQLVESGGGLVQTGGSLRLSCAYSASALRINTMGWYRQVPGKERESVAHITSGDTNYADSVKGRFTISRDNAKNTVTLQMNSLKPEDTAVYYCHALGRSRWGATYDYWGQGTQVTVSS

SEQ ID NO: 19 - protein, 124 aa, synthetic construct
  REGION 1..124: note = Nb NTCP_66
QVQLVESGGGLVQAGGSLRLSCTASGLPFTSYAMGWFRQAPGKEREFVAGITSSGGSTSLADSVKGRFTISRDNAKNTMYLQMNSLKPDDTANYYCAAQRRWGSRWYFADSDYWGQGTQVTVSS

SEQ ID NO: 20 - protein, 124 aa, synthetic construct
  REGION 1..124: note = Nb NTCP_67
QVQLVESGGGLVQAGGSLRLSCTASGLPFTSYAMGWFRQAPGKEREFVAGITPSGGSTSLADSVKGRFTISRDNAKNTMYLQMNSLKPNDTAVYYCAAQRRWGSRWYFADSDYWGQGTQVTVSS

SEQ ID NO: 21 - protein, 124 aa, synthetic construct
  REGION 1..124: note = Nb NTCP_68
QVQLVESGGGLVQAGGSLRLSCTASGLPFTSYAMAWFRQAPGKEREFVAGINQSGGSTSLADSVKGRFTISRDNAKNTMYLQMNSLKPDDTAVYYCAAERRWGSRWYFADSDYWGQGTQVTVSS

SEQ ID NO: 22 - protein, 124 aa, synthetic construct
  REGION 1..124: note = Nb NTCP_69
QVQLVESGGGLVQAGGSLRLSCTASGLPFTSYAMGWFRQAPGKEREFVAGITPSGGSTSLADSVKGRFTISRDNAKNTMYLQMNSLKPDDTAVYYCAAQRRWGSRWYFADSDYWGQGTQVTVSS

SEQ ID NO: 23 - protein, 124 aa, synthetic construct
  REGION 1..124: note = Nb NTCP_70
QVQLVESGGGLVQAGGSLRLSCTASGLPFTSYAMGWFRQAPGKEREFVAGITSSGGSTSLADSVKGRFTISRDNAKNTMYLQMNSLKPDDTADYYCAAQRRWGTRWYFADSDYWGQGTQVTVSS

SEQ ID NO: 24 - protein, 124 aa, synthetic construct
  REGION 1..124: note = Nb NTCP_71
QVQLVESGGGLVQAGGSLRLSCTASGLPFTSYAMGWFRQAPGKEREFVAGISPSGGSTSLADSVKGRFTISRDNAKNTMYLQMNSLKPDDTAIYYCAAQRRWGTRWYFADSDYWGQGTQVTVSS

SEQ ID NO: 25 - protein, 126 aa, synthetic construct
  REGION 1..126: note = NbNTCP_87 - S55Q variant
QVQLVESGGGLVQAGGSLRLSCAVSGRTTANYNMGWFRQAPGKEREFVAGIKWSQGSTYVADSAKGRFTISRDNAKNSVYLQMDSLKPEDTALYYCAANYYGVSWFLISPSSYDYWGQGTQVTVSS

SEQ ID NO: 26 - protein, 126 aa, synthetic construct
  REGION 1..126: note = NbNTCP_87 - S55E variant
QVQLVESGGGLVQAGGSLRLSCAVSGRTTANYNMGWFRQAPGKEREFVAGIKWSEGSTYVADSAKGRFTISRDNAKNSVYLQMDSLKPEDTALYYCAANYYGVSWFLISPSSYDYWGQGTQVTVSS

SEQ ID NO: 27 - protein, 126 aa, synthetic construct
  REGION 1..126: note = NbNTCP_87 -A30Q variant
QVQLVESGGGLVQAGGSLRLSCAVSGRTTQNYNMGWFRQAPGKEREFVAGIKWSSGSTYVADSAKGRFTISRDNAKNSVYLQMDSLKPEDTALYYCAANYYGVSWFLISPSSYDYWGQGTQVTVSS

SEQ ID NO: 28 - protein, 126 aa, synthetic construct
  REGION 1..126: note = Nb NTCP_87- S104G variant
QVQLVESGGGLVQAGGSLRLSCAVSGRTTANYNMGWFRQAPGKEREFVAGIKWSSGSTYVADSAKGRFTISRDNAKNSVYLQMDSLKPEDTALYYCAANYYGVGWFLISPSSYDYWGQGTQVTVSS

SEQ ID NO: 29 - protein, 126 aa, synthetic construct
  REGION 1..126: note = Nb NTCP_87 - S111R variant
QVQLVESGGGLVQAGGSLRLSCAVSGRTTANYNMGWFRQAPGKEREFVAGIKWSSGSTYVADSAKGRFTISRDNAKNSVYLQMDSLKPEDTALYYCAANYYGVSWFLISPRSYDYWGQGTQVTVSS

SEQ ID NO: 30 - protein, 125 aa, synthetic construct
  REGION 1..125: note = Nb NTCP_91- N27D variant
QVQLVESGGGLVEAGGSLRLSCAASTDLRSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 31 - protein, 125 aa, synthetic construct
  REGION 1..125: note = Nb NTCP_91- N27Q variant
QVQLVESGGGLVEAGGSLRLSCAASTQLRSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 32 - protein, 125 aa, synthetic construct
  REGION 1..125: note = Nb NTCP_91- N27E variant
QVQLVESGGGLVEAGGSLRLSCAASTELRSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 33 - protein, 125 aa, synthetic construct
  REGION 1..125: note = Nb NTCP_91- R29N variant
QVQLVESGGGLVEAGGSLRLSCAASTNLNSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 34 - protein, 125 aa, synthetic construct
  REGION 1..125: note = Nb NTCP_91- R29G variant
QVQLVESGGGLVEAGGSLRLSCAASTNLGSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 35 - protein, 125 aa, synthetic construct
  REGION 1..125: note = Nb NTCP_91- R29S variant
QVQLVESGGGLVEAGGSLRLSCAASTNLSSYAMAWFRQAPGKEREFVSFINWNYGNTRYADSVKGRFTISRDNAKITVYLQMNSLKPEDTAVYYCAAATIGRLAGIDSTTLYDYWGQGTQVTVSS

SEQ ID NO: 36 - protein, 125 aa, synthetic construct
  REGION 1..125: note = Nb NTCP_91-N53D variant
QVQLVESGGGLVEAGGSLRLSCAASTNLRSYAMAWFRQAPGKEREFVSFINWDYGNTRYADSVKGRFTISRDNAKITVYLQMNSLKPEDTAVYYCAAATIGRLAGIDSTTLYDYWGQGTQVTVSS

SEQ ID NO: 37 - protein, 125 aa, synthetic construct
  REGION 1..125: note = Nb NTCP_91- L103N variant
QVQLVESGGGLVEAGGSLRLSCAASTNLRSYAMAWFRQAPGKEREFVSFINWNYGNTRYADSVKGRFTISRDNAKITVYLQMNSLKPEDTAVYYCAAATIGRNAGIDSTTLYDYWGQGTQVTVSS

SEQ ID NO: 38 - protein, 10 aa, synthetic construct
  REGION 1..10: note = 6xHis EPEA tag
HHHHHHEPEA

SEQ ID NO: 39 - protein, 47 aa, Hepatitis B virus
GTNLSVPNPLGFFPDHQLDPAFRANSNNPDWDFNPNKDHWPEANKVG

SEQ ID NO: 40 - protein, 5 aa, synthetic construct
  REGION 1..5: note = CDR1 of Nb 87 (Kabat annotation)
NYNMG

SEQ ID NO: 41 - protein, 17 aa, synthetic construct
  REGION 1..17: note = CDR2 of Nb 87 (Kabat annotation)
GIKWSSGSTYVADSAKG

SEQ ID NO: 42 - protein, 16 aa, synthetic construct
  REGION 1..16: note = CDR3 of Nb 87 (Kabat annotation)
NYYGVSWFLISPSSYD

SEQ ID NO: 43 - protein, 5 aa, synthetic construct
  REGION 1..5: note = CDR1 of Nb 91(Kabat annotation)
SYAMA

SEQ ID NO: 44 - protein, 17 aa, synthetic construct
  REGION 1..17: note = CDR2 of Nb 91 (Kabat annotation)
FINWNYGNTRYADSVKG

SEQ ID NO: 45 - protein, 16 aa, synthetic construct
  REGION 1..16: note = CDR3 of Nb 91 (Kabat annotation)
ATIGRLAGIDSTTLYD

SEQ ID NO: 46 - protein, 17 aa, synthetic construct
  REGION 1..17: note = CDR2 of Nb_NTCP_87 - S55Q variant
GIKWSQGSTYVADSAKG

SEQ ID NO: 47 - protein, 17 aa, synthetic construct
  REGION 1..17: note = CDR2 of Nb_NTCP_87 - S55E variant
GIKWSEGSTYVADSAKG

SEQ ID NO: 48 - protein, 16 aa, synthetic construct
  REGION 1..16: note = CDR3 of Nb_NTCP_87- S104G variant
NYYGVGWFLISPSSYD

SEQ ID NO: 49 - protein, 16 aa, synthetic construct
  REGION 1..16: note = CDR3 of Nb NTCP_87 - S111R variant
NYYGVSWFLISPRSYD

SEQ ID NO: 50 - protein, 17 aa, synthetic construct
  REGION 1..17: note = CDR2 of Nb_NTCP_91-N53D variant
FINWDYGNTRYADSVKG

SEQ ID NO: 51 - protein, 16 aa, synthetic construct
  REGION 1..16: note = CDR3 of Nb_NTCP_91- L103N variant
ATIGRNAGIDSTTLYD

SEQ ID NO: 52 - protein, 326 aa, Homo sapiens
AHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEE

SEQ ID NO: 53 - protein, 327 aa, synthetic construct; identical to SEQ ID NO: 3
  REGION 1..327: note = NTCP_CO without N-terminal Met

SEQ ID NO: 54 - protein, 326 aa, synthetic construct
  REGION 1..326: note = NTCP_EM without N-terminal Met
AHTASAPFTFTLPPNFGKRPTDLALSVILVVMLFIIMLSLGCTMEFSKIKAHLWKPKGLAIALVAQYGIMPLTAFVLGKVFRLNNIEALAILICGCSPGGNLSNIFSLAMKGDMNLSIVMTTCSTFLALGMMPLLLYIYSRGIYDGDLKDKVPYKGIVISLVLVLIPCTIGIVLKSKRPQYMRYVIKGGMIIILLCSVAVTVLSAINVGKSIMFAMTPHLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEE

SEQ ID NO: 55 - protein, 326 aa, Homo sapiens; identical to SEQ ID NO: 52

SEQ ID NO: 56 - protein, 326 aa, Canis lupus
APNITAPLNFTLPPNFGKRPTDKALSIILVFLLLIIMLSLGCTMEFSKIKAHFWKPKGLVIALIAQYGIMPLTAFTLGKVFRLNNIEALAILVCGCSPGGTLSNVFSLAMKGDMNLSIVMTTCSTFFALGMMPLLLYIYSNGIYDGDLKDKVPYKGIVSSLVLVLIPCTIGIFLKAKRPQYVRYIKKGGMIIMLLLSVAITALSVINVGKSIRFVMTPHLLATSSLMPFIGFLLGYILSALFRLDGRCSRTVSMETGCQNVQLCSTILNVTFPPEVIGPLFFFPLLYMIFQLGEGVFLISIFRCYEKIKPSKDKTKMIYTAAATEE

SEQ ID NO: 57 - protein, 323 aa, Dasypus novemcinctus
ARNASPTLNFTLPPNFGKRPSDLALSVILVFMLIIIMISLGCTMEFSKIKAHIWKPKGLVIALVAQYGIMPLTAFVLGKVFRLNNIEALAILICGCSPGGTLSNVFSLAMKGDMNLSIVMTTCSTFSALGLMPLLLYIYSRGIYDGDLKGKVPYKGIIVSLVLVLIPCTIGIVLSAKRPQYVRYVVKGGMIIMLILSVAIAGLSAINVGKSIMFVMTAHLLATSSLMPFIGFLLGYVLSALLHLDGRGRRTVSMETGCQNVQLCATILNVTFPPEVIGPLFFFPLLYMIFQLGEGLFLIAIFRCYERIKPPKDKTKMIYTDAR

SEQ ID NO: 58 - protein, 326 aa, Bos taurus
AFNESSPFNFSLPHNFGKRPTDRALSVILVIMLLTIMLSLGCTMEFSKIKAHFWRPKGLAVALVAQFGIMPLTAFGLGKFFQLNNVEALAILICGCSPGGNLSNVFALAIKGDMNLSIVMTTCSTFFALGMMPLLLYLYSRGIYDGSLKDKVPYGGIMISLILILIPCTIGIILKSKRPQYVRYVTKGGIILLLLCSVAVVVLSAINVGKSILFVMTPHLLATSSLMPFIGFLLGYLLSALFCLNGRCKRTVSMETGCQNIQLCSTILNVTFPPEVIGPLFFFPLLYMIFQVGEGLLLVAIFRCYEKFKTPRDDTKMTYKVDATEE

SEQ ID NO: 59 - protein, 326 aa, Mus musculus
AHNVSAPFNFSLPPGFGHRATDTALSVILVVMLLLIMLSLGCTMEFSKIKAHFWKPKGVIIAIVAQYGIMPLSAFLLGKVFHLTSIEALAILICGCSPGGNLSNLFTLAMKGDMNLSIVMTTCSSFTALGMMPLLLYIYSKGIYDGDLKDKVPYKGIMLSLVMVLIPCAIGIFLKSKRPHYVPYVLKAGMIITFSLSVAVTVLSVINVGNSIMFVMTPHLLATSSLMPFTGFLMGYILSALFRLNPSCRRTISMETGFQNVQLCSTILNVTFPPEVIGPLFFFPLLYMIFQLAEGLLFIIIFRCYLKIKPQKDQTKITYKAAATED

SEQ ID NO: 60 - protein, 326 aa, Oryzias latipes
IDNDGGLSGNSSMSNTTTFRAMNMTINFLSLAIVFITMISLGCTMEISKIKAHILKPKGVAIALLAQFFIMPLSAFCLAKVLQMDPIKAVTVLICGCCPGGTLSNIFSLIVKGDMNLSIVMTTCSTFAALGLMPLLLCILCQGFPGLQKAVPYVGIVTTILCTLLASTIGILINHYKPKYSLIVKKTGLWILLISSITLFILSAYVVKDVLWMIVMADLLTAAALMPLIGFTLGYVLSVICKLSPQCSRTVSMETGFQNIQLCTAILKVAFPAQVIGPMFLFPLIYVAFQCSEAFLMALCFTSYRTLKPPAGDKDTNKYADAKQEE

SEQ ID NO: 61 - protein, 324 aa, Homo sapiens
NATVCSGASCVVPESNFNNILSVVLSTVLTILLALVMFSMGCNVEIKKFLGHIKRPWGICVGFLCQFGIMPLTGFILSVAFDILPLQAVVVLIIGCCPGGTASNILAYWVDGDMDLSVSMTTCSTLLALGMMPLCLLIYTKMWVDSGSIVIPYDNIGTSLVSLVVPVSIGMFVNHKWPQKAKIILKIGSIAGAILIVLIAVVGGILYQSAWIIAPKLWIIGTIFPVAGYSLGFLLARIAGLPWYRCRTVAFETGMQNTQLCSTIVQLSFTPEELNVVFTFPLIYSIFQLAFAAIFLGFYVAYKKCHGKNKAEIPESKENGTEPE

SEQ ID NO: 62 - protein, 324 aa, Canis lupus
NATVCNGASCVVAQNNFNDILSVVLSTVLTILLAMVMFSMGCNVEIKKFLGHIKRPWGICVGFLCQFGIMPLTGFILSVAFDILPLQAVVVLIMGCCPGGTASNILAYWVDGDMDLSISMTTCSTLLALGMMPLCLFIYTKMWVDSGTIVIPFDNIGTSLVALVVPVSIGMLVNHKWPQKAKIILKVGSITGAILIVLIAVVGGILYQSAWIIAPKLWIIGTLFPLAGYSLGFLLARISGQSWHRCRTVALETGMQNTQLCSTIVQLSFTQEELNVVFTFPLIYSIFQLAFAAIFLGIYVAYKKCYEKNNAEFPESKDNETVSE

SEQ ID NO: 63 - protein, 317 aa, Dasypus novemcinctus
NATVCDGDSCVLPESNFNQILSIIMSTVLTIMLALVMFSMGCNVDIKKFLGHIKRPWGICVGFLCQFGIMPLTGFILSVAFDILPIQAVVVLIIGCCPGGTSSNILAYWADGDMDLSVSMTTCSTLLALGMMPLCLYIYTKMWTDTGTIIIPYDSIGTSLVALVVPVSIGMFVNHKWPEKAKIILKIGSIAGIFLIVLIAVIGGVLYQGSWVINPKLWIIGTIFPMAGXLLGFLLARIAGQSWHRCRTVALETGMQNTQLCSTIVQLSFTPEQLNHMFTFPLIYSVFQITFAAIFLGIYLAYKKCYGKSSRISEEQK

SEQ ID NO: 64 - protein, 324 aa, Bos taurus
NATVCNGTSCLVPESNFNAILSTILSAVLTILLALVMFSMGCNVELKKFLGHIKRPWGICVGFLCQFGIMPLTGFILSVAFDIMPIQAVVVLIMGCCPGGTASNILAYWVDGDMDLSVSMTTCSTLLALGMMPLCLLIYTKMWVDSGMIVIPYDNIGISLVALVVPVSLGMYVNHKWPQKAKIILKIGSITGALLIVLIAVVGGVLYRSAWIIQPRLWIIGTIFPIAGYSLGFFLARIAGQSWHRCRTVALETGMQNTQLCSTIVQLSFTPEELNLIFTFPLIYSIFQIIAAALFLAVYVVYKKYYRKNNVESPEGRDNEPVPK

SEQ ID NO: 65 - protein, 324 aa, Mus musculus
NATVCEGDSCVVPESNFNAILNTVMSTVLTILLAMVMFSMGCNVEVHKFLGHIKRPWGIFVGFLCQFGIMPLTGFILSVASGILPVQAVVVLIMGCCPGGTGSNILAYWIDGDMDLSVSMTTCSTLLALGMMPLCLFVYTKMWVDSGTIVIPYDSIGISLVALVIPVSFGMFVNHKWPQKAKIILKIGSITGVILIVLIAVIGGILYQSAWIIEPKLWIIGTIFPIAGYSLGFFLARLAGQPWYRCRTVALETGMQNTQLCSTIVQLSFSPEDLNLVFTFPLIYTVFQLVFAAVILGIYVTYRKCYGKNDAEFLEKTDNEMDSR

SEQ ID NO: 66 - protein, 297 aa, Oryzias latipes
DAAICSGTSCLAPANNFNAVLSVVMSTVLTVMLAMVMFAMGCTVDFWKLWGHIRRPWGIVIGFICQFGIMPFTAFALSLAFNVLPVQAIVIIIMGCCPGGSSSNIICYWLDGDMDLSISMTACSSILALGMMPLCLFIYTSVWTSSDSIMIPFDSIGITLAALLIPIAAGMVVKKKWPSQAKKILKVGSVVGFALIIIIAVVGGVLYQSSWTISPSLWIIGAIYPFVGFGLGFLLARFVGQPWDKCRTIALETGFQNSQLCSTIVQLSFSQEELEIMFAFPLIYSIFQLAAAVVSVG

SEQ ID NO: 67 - protein, 272 aa, synthetic construct
  REGION 1..272: note = bivalent Nb87-(GGGGS)x4-Nb87 amino acid sequence
QVQLVESGGGLVQAGGSLRLSCAVSGRTTANYNMGWFRQAPGKEREFVAGIKWSSGSTYVADSAKGRFTISRDNAKNSVYLQMDSLKPEDTALYYCAANYYGVSWFLISPSSYDYWGQGTQVTVSSGGGGSGGGGSGGGGSGGGGSQVQLVESGGGLVQAGGSLRLSCAVSGRTTANYNMGWFRQAPGKEREFVAGIKWSSGSTYVADSAKGRFTISRDNAKNSVYLQMDSLKPEDTALYYCAANYYGVSWFLISPSSYDYWGQGTQVTVSS

SEQ ID NO: 68 - protein, 21 aa, synthetic construct
  REGION 1..21: note = N-terminal Signal peptide for expression of bivalent Nbs
METDTLLLWVLLLWVPGSTGD

SEQ ID NO: 69 - protein, 109 aa, synthetic construct
  REGION 1..109: note = human_IGHV3-JH_consensus
EVQLVESGGGLVQPGGSLRLSCAASGFTFSSYAMHWVRQAPGKGLEWVSVISSDGSSTYYADSVKGRFTISRDNSKNTLYLQMNSLRAEDTAVYYCARWGQGTLVTVSS

SEQ ID NO: 70 - protein, 126 aa, synthetic construct
  REGION 1..126: note = humanized variant Nb87-v1
EVQLVESGGGLVQPGGSLRLSCAVSGRTTANYNMGWFRQAPGKEREFVAGIKWSSGSTYVADSAKGRFTISRDNAKNTVYLQMNSLRPEDTALYYCAANYYGVSWFLISPSSYDYWGQGTLVTVSS

SEQ ID NO: 71 - protein, 126 aa, synthetic construct
  REGION 1..126: note = humanized variant Nb87-v2
EVQLVESGGGLVQPGGSLRLSCAVSGRTTANYNMGWFRQAPGKEREFVAGIKWSSGSTYVADSVKGRFTISRDNAKNTVYLQMNSLRPEDTALYYCAANYYGVSWFLISPSSYDYWGQGTLVTVSS

SEQ ID NO: 72 - protein, 126 aa, synthetic construct
  REGION 1..126: note = humanized variant Nb87-v3
EVQLVESGGGLVQPGGSLRLSCAVSGRTTANYNMGWFRQAPGKEREFVAGIKWSSGSTYYADSAKGRFTISRDNAKNTVYLQMNSLRPEDTALYYCAANYYGVSWFLISPSSYDYWGQGTLVTVSS

SEQ ID NO: 73 - protein, 126 aa, synthetic construct
  REGION 1..126: note = humanized variant Nb87-v4
EVQLVESGGGLVQPGGSLRLSCAVSGRTTANYNMGWFRQAPGKEREFVAGIKWSSGSTYYADSVKGRFTISRDNAKNTVYLQMNSLRPEDTALYYCAANYYGVSWFLISPSSYDYWGQGTLVTVSS

SEQ ID NO: 74 - protein, 125 aa, synthetic construct
  REGION 1..125: note = humanized variant Nb91-v1
EVQLVESGGGLVQPGGSLRLSCAASTNLRSYAMAWFRQAPGKEREFVSFINWNYGNTRYADSVKGRFTISRDNAKITVYLQMNSLRPEDTAVYYCAAATIGRLAGIDSTTLYDYWGQGTLVTVSS

SEQ ID NO: 75 - protein, 125 aa, synthetic construct
  REGION 1..125: note = humanized variant Nb91-v2
EVQLVESGGGLVQPGGSLRLSCAASTNLRSYAMAWFRQAPGKEREFVSFINWNYGNTRYADSVKGRFTISRDNAKNTVYLQMNSLRPEDTAVYYCAAATIGRLAGIDSTTLYDYWGQGTLVTVSS

SEQ ID NO: 76 - protein, 543 aa, synthetic construct
  REGION 1..543: note = AntiC2mCherryXL fusion protein
METDTLLLWVLLLWVPGSTGDQVQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSGGGGSGGGGSGGGGSGGGGSQVQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSSGSGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGSEASSERMYPEDGALKGEVKYRLKLKDGGHYDAEVKTTYKAKKPVQLPGAYNVNRKLDITSHNEDYTIVEQYERAEGRHSTGGMDELYKHHHHHHEPEA

SEQ ID NO: 77 - protein, 4 aa, synthetic construct
LALA

SEQ ID NO: 78 - protein, 6 aa, synthetic construct
LALAPG

SEQ ID NO: 79 - protein, 4 aa, synthetic construct
GGGS

(19) United States
(12) Patent Application Publication
(10) Pub. No.: US 2025/0304664 A1
(43) Pub. Date: Oct. 2, 2025

(54) Human NTCP Binders For Therapeutic Use And Liver-Specific Targeted Delivery

(71) Applicants: VIB VZW, Gent (BE); Vrije Universiteit Brussel, Brussel (BE); Institut Pasteur, Paris Cedex 15 (FR); Centre National De La Recherche Scientifique, Paris Cedex 16 (FR)

(72) Inventors: Jan Steyaert, Beersel (BE); Els Pardon, Wezemaal (BE); Nicolas Reyes, Pessac (FR); Kapil Goutam, Pessac (FR); Francesco Simone lelasi, Grenoble (FR)

(21) Appl. No.: 18/728,315

(22) PCT Filed: Jan. 12, 2023
(86) PCT No.: PCT/EP2023/050615

§ 371 Date: Jul. 11, 2024

(30) Foreign Application Priority Data
Jan. 12, 2022 (EP) 22151078.7

(51) Int. Cl. C07K 16/18; G01N 33/68

(52) U.S. Cl. CPC C07K 16/18; G01N 33/6854; C07K 2317/24; C07K 2317/35; C07K 2317/569; C07K 2317/76

(57) ABSTRACT

The disclosure relates to binding agents and compositions specifically binding the human Na+-taurocholate co-transporting polypeptide (NTCP/SLC10A1), thereby stabilizing a NTCP conformational state which allosterically inhibits its bile salt transport function. More specifically, the disclosure discloses Nanobodies that lock an inward-facing or open-pore NTCP conformational state, thereby useful as novel hepatitis virus B (HBV) and/or hepatitis virus D (HDV) antiviral, for treatment of liver disease, or as vehicle for targeted-delivery to the liver. Finally, the disclosure relates to a screening assay wherein said Nanobodies are used as a tool to identify NTCP conformation-selective compounds with therapeutic potential.

### CROSS-REFERENCE TO RELATED APPLICATIONS

This application is a national phase entry under 35 U.S.C. § 371 of International Patent Application PCT/EP2023/050615, filed Jan. 12, 2023, designating the United States of America and published in English as International Patent Publication WO 2023/135198 on Jul. 20, 2023, which claims the benefit under Article 8 of the Patent Cooperation Treaty to European Patent Application Serial No. 22151078.7, filed Jan. 12, 2022, the entireties of which are hereby incorporated by reference.

### INCORPORATION BY REFERENCE

The ST.26 XML Sequence listing named “10488-10574-US-Substitute Sequence Listing ST26°, created on Feb. 4, 2025, and having a size of 104,471 bytes, is hereby incorporated herein by this reference in its entirety.

### FIELD OF THE INVENTION

The application relates to binding agents and compositions specifically binding the human Na⁺-taurocholate co-transporting polypeptide (NTCP/SLC10A1), thereby stabilizing a NTCP conformational state which allosterically inhibits its sodium ion/bile acid (bile salt) transport function. More specifically, the application discloses Immunoglobulin single variable domains (ISVDs) that lock an inward-facing or open-pore NTCP conformational state, thereby useful as novel hepatitis virus B (HBV) and/or hepatitis virus D (HDV) antiviral, for treatment of liver disease, or as vehicle for targeted-delivery to the liver. Finally, the disclosure relates to a screening assay wherein said ISVDs are used as a tool to identify NTCP conformation-selective compounds with therapeutic potential.

### INTRODUCTION

Bile salts (BSs, also referred to as Na⁺/bile acids) are essential molecules for absorption of lipophilic nutrients and vitamins (vitamin A, D, E, K) in the small intestine, as well as to maintain endocrine and cholesterol homeostasis, and to excrete toxins¹. The vast majority of body BSs pool (˜90%) is recycled daily, shuttling between intestine and liver, where BSs are used to aid nutrient absorption and generate bile, respectively. The liver takes up bile salts (BSs) from blood to generate bile, enabling absorption of lipophilic nutrients, and excretion of metabolites and drugs¹. Human members of the solute carrier 10 (SLC10) family are key BSs transporters to maintain so-called enterohepatic circulation^{5,6}: Na⁺-taurocholate co-transporting polypeptide (NTCP or SLC10A1)⁷ is mainly expressed in the hepatocyte basolateral membrane, and constitutes the main active transport route of BSs into the liver from blood, while apical sodium-dependent bile acid transporter (ASBT or SLA10A2)⁸ expressed in ileum enterocytes takes up BSs from the intestinal lumen. Structural insights into the transport mechanism of NTCP and ASBT come from early X-ray crystal structures of prokaryotic homologs that revealed a 10-transmembrane helix(TM) topology, arranged into so-called core and panel domains^{27,28}. The homologs follow an alternating-access transport mechanism, in which relative movements of the two domains provide alternating access to substrate and sodium binding sites to opposite sides of the membrane. Both transporters are important pharmacological targets, as they can be used to facilitate oral adsorption (ABST)^{9,10} and liver uptake of drugs conjugated to BSs (NTCP)^{11,12}, as well as are involved in the action mechanism (ASBT)¹³ and pharmacokinetics (NTCP)^{14,15} of lowering-cholesterol therapies. Moreover, NTCP downregulation in mice models is associated to increased cholesterol and phospholipid excretion¹⁶, as well as decreases weight gain during high-fat diet¹⁷.

NTCP is also the cellular-entry receptor of human hepatitis B and D viruses (HBV/HDV)^{2,3}, and has emerged as an important antiviral-drug target⁴. However, the molecular mechanisms underlying NTCP transport and viral-receptor functions remain incompletely understood. Chronic HBV infection is a major cause of hepatocellular carcinoma and liver cirrhosis that affects ˜250 million people globally^{18,19}. The viruses use the myristoylated and unstructured N-terminal domain in the large envelope protein, namely preS1 domain (myr-preS1), to recognize and bind human NTCP²⁰⁻²², explaining viral hepatotropism and narrow range of animal hosts. Consistently, myristoylated peptides encompassing the N-terminal 2-48 residues of myr-preS1 (myr-preS1₄₈) act as potent HBV/HDV cell-entry inhibitors²³⁻²⁶. For instance, the preS1-derived peptide myrcludex-b (Gilead) is clinically available to treat chronic hepatitis delta virus (HDV) infection in plasma (or serum) of HDV-RNA positive adult patients with compensated liver disease. Moreover, the monoclonal antibody N6HB426-20 was recently shown to be capable of inhibiting HBV infection of NTCP⁺ hepatoma cell lines, while NTCP interaction with N6HB426-20 does not block or reduce bile acid uptake⁸⁷.

Finally, several important liver diseases require drug-delivery targeted into hepatocytes, which may be mediated using NTCP-specific binders. So there is a need to develop highly specific and selective binders for NTCP that are useful as drug delivery vehicle, as biological for use in liver diseases, including to treat pathologies that gain from blocking NTCP-specific BS transport, or as an antiviral for treatment of chronic HBV and/or HDV infection.

### SUMMARY

The invention relates to binding agents specifically interacting with human NTCP. The binding agents disclosed herein are based on the initial selection of immunoglobulin single variable domain (ISVD) binders, comprising an antigen-binding domain, which bind to a conformational epitope on the transporter as to enable structural analysis of different NTCP conformational states. Remarkably, two ISVDs used in complex with NTCP for cryo-electron microscopy (cryo-EM) analysis revealed unexpected conformational transitions of NTCP. In particular, resolving the human NTCP cryo-EM structures in complexes with the NTCP-specific conformation-selective Nb91 (SEQ ID NO:7) resulted in a conformational state wherein the inward facing conformation of NTCP is blocked, and an open pore state is stabilized, and in complex with an NTCP-specific Nb87 (SEQ ID NO:5) resulted in a stabilized conformation of the inward-facing state, wherein the interaction with preS1-derived peptides is blocked, thereby providing for a binding agent capable to disrupt viral entry.

Both conformations were shown to be key to the NTCP transport cycle with a conformational transition whereby—in complex with Nb91—it opens a wide transmembrane pore (“open pore° state) that serves as the transport pathway for BSs, and discloses key determinant residues for HBV/HDV binding to the outside; and whereby—in complex with Nb87—it stabilizes pore closure and “inward-facing° state impairing recognition of HBV/HDV receptor-binding domain preS1. Both Nb_{NTCP}-NTCP complexes resulted in an inhibited transport of BS substrate, through the stabilization of said specific NTCP conformations, without competing with or sterically hindering of substrate binding sites, providing thus for two types of allosteric inhibitors of human NTCP transport activity, one type by stabilizing an open pore conformation, and a second type by stabilizing an inward-facing conformation.

Moreover, the Nb-NTCP conformational states disclosed herein are the first complexes demonstrating binding selectivity of the viruses for open-to-outside over inward-facing conformations of NTCP transport cycle. Moreover, the unprecedented molecular insights into NTCP “gated-pore° transport and HBV/HDV receptor-recognition mechanisms revealed by the Nb-NTCP complex structures disclosed herein enable progress of the NTCP pharmacological potential in liver-disease therapy. Finally, since Nbs in itself are small, conformation-selective and highly specific, and human NTCP expression is limited to the basolateral (sinusoidal) membrane of hepatocytes, the binding agents disclosed herein further provide for novel liver-specific targeted delivery tools.

In a first aspect, the invention relates to a human Na+-taurocholate co-transporting polypeptide (NTCP) or solute carrier 10 A1 (SLC10A1)-specific binders which comprise an antigen-binding domain responsible for said NTC-specific interaction and which are thereby allosteric inhibitors of the bile salt transport activity of NTCP, as for example determined in a fluorescent substrate-analog transport assay. In one embodiment the NTCP-specific antigen-binding protein-containing binding agents described herein are conformation-selective for one of two conformations described herein, namely the inward-facing state or open pore state, and thereby stabilize one of said conformations, which results in the allosteric inhibition of the bile salt transport of NTCP present in the membrane. The NTCP-specific antigen-binding protein-containing binding agent of the present invention thus locks or stabilizes or predominantly induces the conformation of NTCP in an intermediate transport state shown here as for example the open pore and inward-facing states. In a specific embodiment the NTCP-specific antigen-binding protein-containing binding agent is an allosteric inhibitor of the myr-PreS1 peptide binding, thereby blocking human hepatitis B and/or hepatitis D viral entry, more specifically said allosteric inhibition is not mediated via steric hindrance of myr-PreS1 binding but by locking the NTCP protein in a novel inward-facing conformational state.

In a further specific embodiment said NTCP-specific antigen-binding protein-containing binding agent is an allosteric inhibitor of Bile acid or bile salt transport wherein the antigen-binding protein comprises an antibody, an antibody mimetic, a single domain antibody, an immunoglobulin single variable domain (ISVD), a Nanobody, a VHH. In a specific embodiment, said NTCP-specific antigen-binding protein-containing binding agent described herein is a binding agent comprising an antigen-binding protein comprising or consisting or an ISVD, alternatively comprising one or more ISVDs, wherein said ISVD specifically binds the human NTCP via its antigen-binding domain, and provides for allosteric inhibition of NTCP bile salt transport by stabilizing the inward-facing or the open pore conformational state of NTCP.

A further embodiment relates to the NTCP-specific antigen-binding protein-containing binding agent described herein, comprising an ISVD which upon binding to NTCP stabilizes or locks an ‘inward-facing’ conformational state. In a specific embodiment, said NTCP-specific binding agent stabilizing an ‘inward-facing’ conformational state comprises an ISVD sequence wherein the CDRs are as presented in any of SEQ ID NOs: 5, 6, 14, or 19-29, wherein the CDRs are annotated according to Kabat, MacCallum, IMGT, AbM, or Chothia, as defined also further herein. In another specific embodiment, said NTCP-specific binding agent stabilizing an ‘inward-facing’ conformational state comprises an ISVD sequence wherein the CDRs are presented as CDR1 comprising SEQ ID NO:40, CDR2 comprising SEQ ID NO:41, 46 or 47, and CDR3 comprising SEQ ID NO: 42, 48, or 49. In a further specific embodiment, the NTCP-specific binding agent described herein, comprises at least one ISVD comprising a sequence selected from the group of sequences of SEQ ID NOs: 5, 6, 14, or 19-29, or a functional variant of any one thereof with at least 90% identity over the full length of the ISVD sequence wherein the non-identical amino acids are located in one or more Framework residues, or a humanized variant of any of SEQ ID NOs: 5, 6, 14, or 19-29. In a further specific embodiment said NTCP-specific antigen-binding protein-containing binding agent comprises a humanized variant of the ISVD referred to herein as Nb87, as shown in any one of SEQ ID No: 5, or 25-29, wherein said humanized variant comprises one or more amino acid substitutions, preferably limited to substitutions in the framework regions, even more preferably selected from substituting amino acid residues selected from any one of the positions corresponding to Q1, A14, V59, A63, S77, D82a, K83, or Q108 (according to Kabat numbering) of SEQ ID NO: 5, more preferably a humanized variant of Nb87 wherein any one or more of those residues is substituted selected from Q1E, A14P, V59Y, A63V, S77T, D82aN, K83R, or Q108L, or any combination thereof, or even more preferably as presented in SEQ ID NO: 70-73.

A further embodiment relates to the NTCP-specific antigen-binding protein-containing binding agent described herein, comprising an ISVD which upon binding to NTCP stabilizes or locks an ‘open pore’ conformational state. In a specific embodiment, said NTCP-specific antigen-binding protein-containing binding agent stabilizing an ‘open pore’ conformational state comprises an ISVD sequence wherein the CDRs are as presented in any of SEQ ID NOs: 7-13, 15-18 or 30-37, wherein the CDRs are annotated according to Kabat, MacCallum, IMGT, AbM, or Chothia, as defined also further herein. In another specific embodiment, said NTCP-specific antigen-binding protein-containing binding agent stabilizing an ‘open pore’ conformational state comprises an ISVD sequence wherein the CDRs are presented as CDR1 comprising SEQ ID NO:43, CDR2 comprising SEQ ID NO:44 or 50, and CDR3 comprising SEQ ID NO: 45 or 51. In a further specific embodiment, the NTCP-specific binding agent described herein comprises at least one ISVD comprising a sequence selected from the group of sequences of SEQ ID NOs: 7-13, 15-18 or 30-37, or a functional variant of any one thereof with at least 90% identity over the full length of the ISVD sequence wherein the non-identical amino acids are located in one or more Framework residues, or a humanized variant of any of SEQ ID NOs: 7-13, 15-18 or 30-37. In a further specific embodiment said NTCP-specific antigen-binding protein-containing binding agent comprises a humanized variant of the ISVD referred to herein as Nb91, as shown in any one of SEQ ID NO: 7, or 30-37, wherein said humanized variant comprises one or more amino acid substitutions, preferably limited to substitutions in the framework regions, even more preferably selected from substituting amino acid residues selected from any one of the positions corresponding to Q1, E13, A14, I76, K83, or Q108, (according to Kabat numbering) of SEQ ID NO: 7, more preferably a humanized variant of Nb91 wherein any one or more of those residues is substituted selected from Q1E, E13Q, A14P, I76N, K83R, or Q108L, or any combination thereof, or even more preferably as presented in SEQ ID NO: 74-75.

In another embodiment, the NTCP-specific antigen-binding protein-containing binding agent as described herein is a multivalent or multispecific agent. The binding moieties within said multivalent or multispecific agent may be directly linked, or fused by a linker or spacer. Alternatively, multivalent or multispecific binding agents as described herein may be formed by fusion head-to-tail, as fusion of the ISVD to an Fc-tail, or as a further chimeric antibody format known in the art.

Another embodiment relates to the NTCP-specific antigen-binding protein-containing binding agent as described herein, comprising at least one ISVD which is an allosteric inhibitor of bile acid transport by locking an NTCP confirmation state that is an inward-facing or open pore state, which is further labelled, tagged, or is conjugated to a further moiety, such as another functional moiety. In a specific embodiment, said conjugated functional moiety may comprise a therapeutic moiety, a half-life extension, a small-molecule compound, an enzyme, an antibody, a genome-editing component, a nucleic acid molecule, or a nanoparticle such as a liposome.

In a further aspect, the invention relates to an isolated nucleic acid molecule encoding the one or more binding agents described herein, or specifically encoding the multivalent or multispecific binding agents, as defined herein. A further embodiment relates to the recombinant vector comprising said nucleic acid molecule.

Another aspect relates to a composition as described herein, which may be a pharmaceutical composition, comprising said one or more NTCP-specific antigen-binding protein-containing binding agents as described herein, or the nucleic acid molecule or vector encoding said binding agent, and said pharmaceutical composition optionally comprising a further therapeutic agent, a diluent, carrier and/or excipient.

Another aspect of the invention relates to the protein complex made by the human NTCP-specific antigen-binding protein-containing binding agent as described herein and human NTCP. Specifically, an embodiment relates to a complex between NTCP_{EM} and a binding agent comprising an ISVD as described herein, more specifically an ISVD comprising Nb_{NTCP}87 or Nb_{NTCP}91 or Mb_{NTCP}91.

The invention likewise relates to an above-described (pharmaceutical) composition, binding agent(s), nucleic acid and/or a recombinant vector, for use as a medicament. The invention likewise relates to an above-described (pharmaceutical) composition, binding agent(s), nucleic acid and/or a recombinant vector, for use in therapeutic treatment of a subject. The invention likewise relates to an above-described (pharmaceutical) composition, binding agent(s), nucleic acid and/or a recombinant vector, for use in the treatment of a human HBV/HDV infection, more specifically a chronic HBV/HDV infection. The invention likewise relates to an above-described (pharmaceutical) composition, binding agent(s), nucleic acid and/or a recombinant vector, for use in treatment of a liver disorder of a subject, or a disorder curable by blocking NTCP bile acid transport. The invention likewise relates to an above-described (pharmaceutical) composition, or binding agent(s), for use as in liver-specific targeted delivery of said agent, optionally including further moieties.

The invention likewise relates to an above-described (pharmaceutical) composition, binding agent(s), nucleic acid and/or a recombinant vector, for use in the manufacture of a diagnostic kit.

In a final aspect, the invention relates to an in vitro method for screening and producing a conformation-selective compound of human NTCP, said method comprising the steps of:
a) contacting the NTCP-specific binding agent as described herein with a sample comprising NTCP, or providing the complex as described herein, and
b) adding a test compound to said mixture of a., under conditions wherein the binding agent is in complex with NTCP and the test compound can bind the complex, and
c) analyze whether the test compound is bound to said complex to identify said compound as a conformation-selective compound specifically binding human NTCP,
wherein human NTCP is present in complex with the binding agent in an inward-facing or an open pore conformational state.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1C. Functional and structural analyses of NTCP_{EM}-Nb complexes.

FIG. 1A, Fluorescent substrate analog (tauro-nor-THCA-24-DBD) uptake in cells expressing NTCP constructs or negative control (excitatory amino acid transporter, EAAT1) using sodium-based (green) and choline-based (grey) buffers, respectively. Bars depict averages of n=5, 7, and 4 biologically independent experiments for NTCP_{WT}, NTCP_{EM}, and EAAT1, respectively. Error bars represent SEM. Circles represent values from individual experiments. FIG. 1B, Tauro-nor-THCA-24-DBD transport inhibition in cells expressing NTCP_{EM} by Nb91 (red) and Nb87 (blue), respectively. Solid lines are fits of a single-site binding equation (see methods). Squares depict averages of n=3 biologically independent experiments, and error bars represent SEM. FIG. 1C, Density corresponding to NTCP_{EM} core (blue) and panel (orange) domains, as well as the nanobody part of Mb91 (green) is shown.

FIGS. 2A-2C. NTCP topology and architecture.

FIG. 2A, Cartoon representation of NTCP topology. FIG. 2B, Structure of NTCP_{EM} in complex with Mb91. Mb91 is not shown for clarity of display. TM2-4 (dark blue) and TM7-9 (light-blue) in the core domain are related by pseudo two-fold symmetry, and panel domain is formed by TM1 and TM5-6 (orange). Polar conserved residues lining the space between core and panel domain (pink), as well as sidechains contributing to Na1 and Na2 (yellow) are shown. FIG. 2C, X-motif formed by unwinding of TM3 and TM8 is displayed. Only TM2 and TM3 (dark blue), and TM8 (light blue) are shown.

FIGS. 3A and 3B. Isomerization between open-pore and inward-facing states.

FIG. 3A, NTCP_{EM} structures in complex with Nb87 (left), and Mb91 (right) adopting inward-facing and open-pore conformations, respectively. Nb87 and Mb91 are not shown. Extra cryo-EM density in the open-pore structure is shown (pink surface), and polar residues in close proximity to the density are labeled in both structures for comparison. FIG. 3B, Molecular surface representation of NTCP_{EM} inward-facing (left) and open-pore (right) structures. Surfaces are colored based on a residue-hydrophobicity scale with green representing most hydrophobic and purple most hydrophilic. The structures have been tilted ˜20° to better display cytoplasmic cavity (left), and open pore (right), respectively.

FIGS. 4A-4D. Nb87 inhibits myr-PreS1 binding.

FIG. 4A, Nb87 (left, cyan surface) and Nb91 (right, green surface) bind overlapping 3D epitopes on the extracellular surface of the core domain, distant from myr-preS1 binding-determinant TM5 region, and residues within the pore (highlighted in pink). In the inward-facing state (left) TM5 packs against the core domain (blue). In the open-pore state (right), the core domain moves outward and away from TM5 exposing important residues for myr-preS1 binding (pink). FIG. 4B, Extracellular view of cross-sections passing through the myr-preS1 binding determinant region in TM5 (highlighted in pink). Inward-facing (top), and open-pore (bottom) show TM5-core domain (blue) interfaces. FIG. 4C, Myr-preS1₄₈-GFP labelling of cells expressing NTCP_{WT} (left) and NTCP_{EM} (right), respectively. Pre-incubation with Nb87, but not with Nb91, impaired myr-preS1₄₈-GFP labelling. Plots depict average of n=3 biologically independent experiments, and circles represent values from individual experiments. Error bars represent SEM. FIG. 4D, Cartoon representation of NTCP “gated-pore° transport mechanism and the relative movements of core (blue), and panel (orange) domains. Myr-preS1 domain of HBV/HDV (green) preferentially binds to open-to-outside states of NTCP transport cycle.

FIGS. 5A and 5B. NTCP consensus designs.

FIG. 5A, Phylogenetic tree of NTCP vertebrate orthologs used to determined consensus amino acids. NCBI protein sequence IDs are given in parenthesis. FIG. 5B, Amino acid sequence alignment of human NTCP (residues 3-328) and consensus designs NTCP_{CO} and NTCP_{EM}.

FIGS. 6A-6G. Cryo-EM data processing pipeline of NTCP_{EM}-Mb91 complex.

FIG. 6A, Representative EM micrograph with examples of individual particles (red circles). 21,390 micrographs were collected. FIG. 6B, Gallery of representative 2D class-averages. FIG. 6C, 3D classes from ab initio classification. FIG. 6D, Non-Uniform refined map FIG. 6E, Local-refinement map after micelle removal, and color coded according to local resolution estimation. FIG. 6F, Viewing direction distribution plot. FIG. 6G, Fourier shell correlation (FSC) plot of local refinement with FSC threshold at 0.143.

FIGS. 7A-7G. Cryo-EM data processing pipeline of NTCP_{EM}-Nb87 complex.

FIG. 7A, Representative EM micrograph with examples of individual particles (red circles). 21,790 micrographs were collected. FIG. 7B, Gallery of representative 2D class-averages. FIG. 7C, 3D classes from ab initio classification. FIG. 7D, Homogenous refinement 3D map. FIG. 7E, Local-refinement map after micelle removal, and color coded according to local resolution estimation. FIG. 7F, Viewing direction distribution plot. FIG. 7G, Fourier shell correlation (FSC) plot of local refinement with FSC threshold at 0.143.

FIG. 8. Cryo-EM density in NTCP_{EM}-Mb91 structure.

Cryo-EM density corresponding to individual NTCP_{EM} transmembrane helices in complex with Mb91.

FIG. 9. Amino acid sequence alignment of NTCP and ASBT vertebrate orthologs.

Squares indicate residues that contribute sidechains to Na1 and Na2, triangles indicate conserved polar residues in NTCP orthologs that line the space between core and panel domains, and circles proline and glycine residues that act as hinges during the isomerization between open-pore and inward-facing states.

FIGS. 10A and 10B. NTCP amino acid conservation surface mapping.

Amino acid conservation across NTCP vertebrate orthologs is mapped into FIG. 10A, NTCP_{EM} inward-facing (left) and FIG. 10B, open-pore (right) structures. Spheres represent alpha-carbon atoms of 7 consensus mutations to improve thermal stability in the construct used for cryo-EM analysis (NTCP_{EM}).

FIGS. 11A-11D. Extra-density in NTCP_{EM} open-pore structures.

FIGS. 11A and 11B, Membrane views of NTCP_{EM}-Mb91 in detergent solutions highlighting extra cryo-EM density in the pore (purple surface), and residues in proximity of the density. FIGS. 11C and 11D, Membrane views of NTCP_{EM}-Nb91 in nanodisc highlighting similar extra cryo-EM density in the pore, and residues in proximity.

FIGS. 12A and 12B. Cryo-EM density and structure of NTCP_{EM}-Nb91 reconstituted in nanodisc.

FIG. 12A, Structure of NTC_{EM}-Nb91 complex reconstituted in nanodisc and the corresponding cryo-EM density. FIG. 12B, Superimposition of NTCP_{EM} structures in complex with Nb91 in nanodisc (dark pink) and that in complex with Mb91 in detergent (core domain, blue; panel domain, orange) (RMSD ˜1.4 Å over all atoms).

FIG. 13. Effect of nanobodies on fluorescent bile salt substrate analog (tauro-nor-THCA-24-DBD) uptake in HEK293 cells expressing NTCP_{CO} construct.

Nanobodies were applied at 1 μM.

FIG. 14. Effect of nanobodies on fluorescent bile salt substrate analog (tauro-nor-THCA-24-DBD) uptake in HEK293 cells expressing NTCP_{WT-KOG} construct.

Nanobodies were applied at 2 μM (Nb87) and 10 μM (Nb53, Nb61, Nb91, Nb83, and Nb79), respectively.

FIG. 15. Effect of nanobodies on myr-preS1₄₈-GFP fusion peptide labelling of HEK293 cells expressing NTCP_{CO}.

Nanobodies were applied at 1 μM.

FIG. 16. Effect of nanobodies on myr-preS1₄₈-GFP fusion peptide labelling of HEK293 cells expressing NTCP_{WT}.

Nanobodies were applied at 10 μM (Nb53 and Nb87) and 50 μM (Nb67), respectively.

FIG. 17. Effect of nanobodies on fluorescent bile salt substrate analog (tauro-nor-THCA-24-DBD) uptake in HEK293 cells expressing NTCP_{WT-KOG} construct.

Competition experiments of single-mutant Nb87 constructs compared to Nb87. All nanobodies were applied at 2 μM.

FIG. 18. Effect of nanobodies on fluorescent bile salt substrate analog (tauro-nor-THCA-24-DBD) uptake in HEK293 cells expressing NTCP_{WT-KOG} construct.

Competition experiments of single-mutant Nb91 constructs compared to Nb91. All nanobodies were applied at 10 μM.

FIG. 19. Nb_{NTCP} amino acid sequences for Nb87 and Nb91 numbered according to Kabat numbering, and CDR annotations indicated according to MacCallum, AbM, Chothia, Kabat and IMGT in grey labeled boxes.

Nb87 corresponding to SEQ ID NO:5, and Nb91 corresponding to SEQ ID NO:7. CDR sequences according to Kabat annotation are also shown for Nb87 and Nb91 in SEQ ID NOs: 40-45.

FIGS. 20A and 20B. Bivalent Nb87 construct expressed in HEK293F cells.

Construct details (FIG. 20A) and Size exclusion chromatographic profile of purified protein (FIG. 20B).

FIGS. 21A and 21B. Surface labelling of NTCP expressing HEK293F cells using AntiC dimer-mCherryXL fusion.

Schematics (FIG. 21A) and Fluorescent microscopic images (FIG. 21B). Nb87 was used as a primary Nb at 20 μM and (AntiC)2-mCherryXL was used at 10 μM.

FIG. 22. Alignment of VHH amino acid sequences for humanization of Nb87 and Nb91.

The human_IGHV3-JH consensus sequence_(SEQ ID NO:69) was aligned with original Nb87, Nb88 and Nb91, and a number of humanized variants Nb87v1-v4 (SEQ ID NO:70-73), as well as humanized variants Nb91v1 and v2 (SEQ ID NOs: 74-75). Residues not present in the Nb sequences or identical to the human_IGHV3-JH consensus corresponding residue are shown as a hyphen or dot, respectively.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

‘Nucleotide sequence’, “DNA sequence° or “nucleic acid molecule(s)° as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps° substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence° is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. With a “chimeric gene° or “chimeric construct” or “chimeric gene construct° is meant a recombinant nucleic acid sequence in which a promoter or regulatory nucleic acid sequence is operatively linked to, or associated with, a nucleic acid sequence that codes for an mRNA, such that the regulatory nucleic acid sequence is able to regulate transcription or expression of the associated nucleic acid coding sequence. The regulatory nucleic acid sequence of the chimeric gene is not operatively linked to the associated nucleic acid sequence as found in nature. An “expression cassette” comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector°.

The terms “protein°, “polypeptide°, and “peptide° are interchangeably used further herein to refer to a polymer of amino acid residues and to variants and synthetic analogues of the same. A “peptide° may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation, and also myristoylation. Based on the amino acid sequence and the modifications, the atomic or molecular mass or weight of a polypeptide is expressed in (kilo)dalton (kDa). A “protein domain° is a distinct functional and/or structural unit in a protein. Usually a protein domain is responsible for a particular function or interaction, contributing to the overall role of a protein. Domains may exist in a variety of biological contexts, where similar domains can be found in proteins with different functions.

As used herein, the term “protein complex° or “complex° or “assembled protein(s)° refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions. A protein complex can be a non-covalent interaction of only proteins, and is then referred to as a protein-protein complex; for instance, a non-covalent interaction of two proteins, of three proteins, of four proteins, etc. More specifically, a complex of a membrane protein, such as NTCP, and another protein of interest, such as a Nb, or a membrane protein and a nucleic acid molecule, optionally with other proteins or compounds bound to it.

By “isolated” or “purified° is meant material that is substantially or essentially free from components that normally accompany it in its native state. For example, an “isolated polypeptide” or “purified polypeptide° refers to a polypeptide which has been purified from the molecules which flank it in a naturally-occurring state, e.g., an antibody or nanobody as identified and disclosed herein which has been removed from the molecules present in the a sample or mixture, such as a production host, that are adjacent to said polypeptide. An isolated protein or peptide can be generated by amino acid chemical synthesis or can be generated by recombinant production or by purification from a complex sample.

The term “linked to°, or “fused to°, as used herein, and interchangeably used herein as “connected to°, “conjugated to°, “ligated to° refers, in particular, to “genetic fusion°, e.g., by recombinant DNA technology, as well as to “chemical and/or enzymatic conjugation resulting in a stable covalent link.

“Homologue°, “Homologues° of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. Preferably, the percentage of identity is calculated over a window of the full length sequence referred to. A “substitution”, or “mutation°, or “variant° as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity, which is hereby defined as a ‘functional variant’.

The term “wild-type° refers to a gene or gene product isolated from a naturally occurring source. A wild-type gene is that which is most frequently observed in a population and is thus arbitrarily designed the “normal° or “wild-type° form of the gene. In contrast, the term “modified°, “mutant°, “engineered° or “variant° refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product.

The term “binding pocket” or “binding site° refers to a region of a molecule or molecular complex, that, as a result of its shape and charge, favourably associates with another chemical entity or binding domain, such as a compound, proteins, peptide, antibody or Nb, among others. For antibody-related molecules, the term “epitope° or “conformational epitope° is also used interchangeably herein. The term “pocket” includes, but is not limited to cleft, channel or site. The human NTCP herein described comprises a binding pocket or binding site which include, but is not limited to a Nanobody binding site. The term “part of a binding pocket/site” refers to less than all of the amino acid residues that define the binding pocket, binding site or epitope. For example, the atomic coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

“Binding° means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,° as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. A “binding agent°, or “agent° as used interchangeably herein, relates to a molecule that is capable of binding to another molecule, via a binding region or binding domain located on the binding agent, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others.

An “epitope°, as used herein, refers to an antigenic determinant of a polypeptide, constituting a binding site or binding pocket on a target molecule, such as human NTCP. Said epitopes may comprise at least one amino acid that is essential for binding the binding agent, though preferably comprise at least 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance, cryo-EM, or other structural analyses. A “conformational epitope°, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein, especially for membrane proteins. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, hydrophobicity, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody° refers to an immunoglobulin (Ig) molecule or a molecule comprising an immunoglobulin (Ig) domain, which specifically binds with an antigen. ‘Antibodies’ can further be intact immunoglobulins derived from natural sources or from recombinant sources and can be immunoreactive portions of intact immunoglobulins. The term “active antibody fragment” refers to a portion of any antibody or antibody-like structure that by itself has high affinity for an antigenic determinant, or epitope, and contains one or more CDRs accounting for such specificity. Non-limiting examples include immunoglobulin domains, Fab, F(ab)′2, scFv, heavy-light chain dimers, immunoglobulin single variable domains, Nanobodies (or VHH antibodies), domain antibodies, and single chain structures, such as a complete light chain or complete heavy chain.

The term “antibody fragment° and “active antibody fragment° or “functional variant° as used herein refer to a protein comprising an immunoglobulin domain or an antigen-binding domain capable of specifically binding human NTCP, more specifically the inward-facing or open pore conformational state of human NTCP. Antibodies are typically tetramers of immunoglobulin molecules. The term “immunoglobulin (Ig) domain°, or more specifically “immunoglobulin variable domain° (abbreviated as) “IVD°) means an immunoglobulin domain essentially consisting of four “framework regions° which are referred to in the art and herein below as “framework region 1° or “FR1°; as “framework region 2° or “FR2°; as “framework region 3° or “FR3°; and as “framework region 4° or “FR4°, respectively; which framework regions are interrupted by three “complementarity determining regions° or “CDRs°, which are referred to in the art and herein below as “complementarity determining region 1° or “CDR1°; as “complementarity determining region 2° or “CDR2°; and as “complementarity determining region 3° or “CDR3°, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain° of this invention refers to “immunoglobulin single variable domains° (abbreviated as “ISVD”), equivalent to the term “single variable domains°, and defines molecules wherein the antigen binding site is present on, and formed by, a single immunoglobulin domain. This sets immunoglobulin single variable domains apart from “conventional° immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains°, also known as VHHs, VHH domains, VHH antibody fragments, and VHH antibodies, have originally been described as the antigen binding immunoglobulin (Ig) (variable) domain of “heavy chain antibodies° (i.e., of “antibodies devoid of light chains°; Hamers-Casterman et al (1993) Nature 363:446-448). The term “VHH domain° has been chosen to distinguish these variable domains from the heavy chain variable domains that are present in conventional 4-chain antibodies (which are referred to herein as “VH domains°) and from the light chain variable domains that are present in conventional 4-chain antibodies (which are referred to herein as “VL domains°). For a further description of VHHs and Nanobody, reference is made to the review article by Muyldermans (Reviews in Molecular Biotechnology 74:277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues° in one or more of the framework sequences. For numbering of the amino acid residues of an IVD different numbering schemes can be applied. For example, numbering can be performed according to the AHo numbering scheme for all heavy (VH) and light chain variable domains (VL) given by Honegger, A. and Pluckthun, A. (J. Mol. Biol. 309, 2001), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids in the article of Riechmann, L. and Muyldermans, S., 231 (1-2), J Immunol Methods. 1999. It should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein. Determination of CDR regions may also be done according to different methods, such as the designation based on contact analysis and binding site topography as described in MacCallum et al. (J. Mol. Biol. (1996) 262, 732-745). Or alternatively the annotation of CDRs may be done according to AbM (AbM is Oxford Molecular Ltd.'s antibody modelling package as described on http://www.bioinf.org.uk/abs/index.html), Chothia (Chothia and Lesk, 1987; Mol Biol. 196:901-17), Kabat (Kabat et al., 1991; 5^{th} edition, NIH publication 91-3242), or IMGT (LeFranc, 2014; Frontiers in Immunology. 5 (22): 1-22). Those annotations exist for numbering amino acids in immunoglobulin protein sequences, though in the present application solely the Kabat numbering is used, or the specific SEQ ID numbering, as indicated. Said annotations further include delineation of CDRs and framework regions (FRs) in immunoglobulin-domain-containing proteins, and are known methods and systems to a skilled artisan who thus can apply these annotations onto any immunoglobulin protein sequences without undue burden. These annotations differ slightly, but each intend to comprise the regions of the loops involved in binding the target.

VHHs or Nbs are often classified in different sequences families or even superfamilies, as to cluster the clonally related sequences derived from the same progenitor during B cell maturation (Deschaght et al. 2017. Front Immunol. 10; 8:420). This classification is often based on the CDR sequence of the Nbs, and wherein for instance each Nb family is defined as a cluster of (clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, having the same effect or functional impact.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution. Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee, and are further clarified in the examples provided herein. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see WO2008/020079 Tables A-05-A08; all numbering according to the Kabat). Humanization typically only concerns substitutions in the FR and not in the CDRs, as this could/would impact binding affinity to the target and/or potency.

The composition or binding agent(s) of the invention as described herein may appear in a “multivalent° or “multispecific° form and thus be formed by bonding, chemically or by recombinant DNA techniques, together two or more identical or different binding agents. Said multivalent forms may be formed by connecting the building block directly or via a linker, or through fusing the with an Fc domain encoding sequence. Non-limiting examples of multivalent constructs include “bivalent° constructs, “trivalent° constructs, “tetravalent° constructs, and so on. The immunoglobulin single variable domains comprised within a multivalent construct may be identical or different, preferably binding to the same or overlapping binding site. In another particular embodiment, the binding agent(s) of the invention are in a “multi-specific° form and are formed by bonding together two or more building blocks or agents, of which at least one binds to human NTCP, as shown herein, and at least one binds to a further target or alternative molecule, so when present in multispecific fusion, presenting a binding agent or composition that is capable of specifically binding both epitopes or targets, thus comprising binders with a different specificity. Non-limiting examples of multi-specific constructs include “bi-specific° constructs, “tri-specific° constructs, “tetra-specific° constructs, and so on. To illustrate this further, any multivalent or multi-specific (as defined herein) ISVD of the invention may be suitably directed against two or more different epitopes on the same NTCP antigen, or may be directed against two or more different antigens, for example against human NTCP and one as a half-life extension against Serum Albumin or SpA, or another target. Multivalent or multi-specific ISVDs of the invention may also have (or be engineered and/or selected for) increased avidity and/or improved selectivity for the desired NTCP interaction, and/or for any other desired property or combination of desired properties that may be obtained by the use of such multivalent or multi-specific immunoglobulin single variable domains. Upon binding human NTCP, said multi-specific binding agent or multivalent ISVD may have an additive or synergistic impact on the binding and/or therapeutic effect on NTCP, such as blocking HBV/HDV viral entry or bile acid/salt transport. In another embodiment, the invention provides a multispecific binding agent which specifically binds NTCP as to direct said binding agent to the surface of hepatocytes specifically expressing NTCP, and specifically targeting a further target when present in the liver, i.e. using the ISVDs specific for NTCP as a vehicle for targeted delivery of therapeutic moieties herein. In another embodiment, the invention provides a polypeptide comprising any of the immunoglobulin single variable domains according to the invention, either in a monovalent, multivalent or multi-specific form. Thus, polypeptides comprising monovalent, multivalent or multi-specific nanobodies are included here as non-limiting examples. The multivalent or multispecific binders or building blocks may be fused directly or fused by a suitable linker, as to allow that the at least two different binding sites can be reached or bound simultaneously by the multispecific agent. Alternatively, at least one ISVD as described herein may be fused at its C-terminus to an Fc domain, for instance an Fc-tail of an Ig, resulting in a protein binding agent of bivalent format wherein two of said VHH-Ig Fcs, or humanized forms thereof, form a heavy chain only-antibody-type molecule through disulfide bridges in the hinge region of the Fc part. Said humanized forms thereof, such as IgG humanized forms, include but are not limited to the IgG humanization variants known in the art, for instance to modulate Fc-mediated effector functions, including variants with for instance C-terminal deletion of Lysine, alteration or truncation in the hinge region, LALA or LALAPG mutations as described^{88,89}, among other substitutions in the IgG sequence. In an alternative setup, an Fc fusion is designed by linking the C-terminus of such a bivalent or bispecific binder fused by a linker to an Fc domain, which then upon expression in a host form a multivalent or multispecific-antibody-type molecule through disulfide bridges in the hinge region of the Fc part.

As used herein, a “therapeutically active agent° or “therapeutically active composition° means any molecule or composition of molecules that has or may have a therapeutic effect (i.e. curative or prophylactic effect) in the context of treatment of a disease (as described further herein). Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The binding agent or the composition, or pharmaceutical composition of the invention may act as a therapeutically active agent, when beneficial in treating patients infected with HBV/HDV viral infections, or patients suffering from another liver disease. The therapeutically active agent/binding agent or composition may include an agent comprising an ISVD specifically binding the human NTCP inward-facing or open pore conformational state as defined herein, and/or may contain or be coupled to additional functional groups, or functional moieties advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, PA (1980). Such functional groups may for example be linked directly (for example covalently) to the ISVD, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain may comprise the engineering into bifunctional or bispecific domains (for example, one ISVD or active antibody fragment against the human NTCP and one against a serum protein such as albumin aiding in prolonging half-life) or into fusions of antibody fragments, in particular immunoglobulin single variable domains, with peptides (for example, a peptide against a serum protein such as albumin).

As used herein, the terms “determining,” “measuring,” “assessing,”, “identifying°, “screening°, and “assaying” are used interchangeably and include both quantitative and qualitative determinations. “Similar° as used herein, is interchangeable for alike, analogous, comparable, corresponding, and -like or alike, and is meant to have the same or common characteristics, and/or in a quantifiable manner to show comparable results i.e. with a variation of maximum 20%, 10%, more preferably 5%, or even more preferably 1%, or less.

The term °subject°, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated °patient° herein. However, it will be understood that the aforementioned terms do not imply that symptoms are present.

The term “medicament°, as used herein, refers to a substance/composition used in therapy, i.e., in the prevention or treatment of a disease or disorder. According to the invention, the terms “disease° or “disorder° refer to any pathological state, in particular to the diseases or disorders as defined herein.

The term “treatment° or “treating° or “treat° can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders. Therapeutic treatment is thus designed to treat an illness or to improve a person's health, rather than to prevent an illness. Treatment may also refer to a prophylactic treatment which relates to a medication or a treatment designed and used to prevent a disease from occurring.

A “composition° relates to a combination of one or more active molecules, and may further include buffered solutions and/or solutes such as pH buffering substances, water, saline, physiological salt solutions, glycerol, preservatives, etc. for which a person skilled in the art is aware of the suitability to obtain optimal performance. Suitable conditions as used herein could also refer to suitable binding conditions, for instance when Nbs or test compounds are aimed to bind human NTCP.

A pharmaceutical composition comprising the one or more binding agents or therapeutic agents, or recombinant vector as provided herein, optionally comprise a carrier, diluent or excipient. A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient°, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles. A pharmaceutically effective amount of polypeptides, or conjugates of the invention and a pharmaceutically acceptable carrier is preferably that amount which produces a result or exerts an influence on the particular condition being treated. For therapy, the pharmaceutical composition of the invention can be administered to any patient in accordance with standard techniques. The administration can be by any appropriate mode, including orally, parenterally, topically, nasally, ophthalmically, intrathecally, intracerebroventricularly, sublingually, rectally, vaginally, and the like. Still other techniques of formulation as nanotechnology and aerosol and inhalant are also within the scope of this invention. The dosage and frequency of administration will depend on the age, sex and condition of the patient, concurrent administration of other drugs, counter-indications and other parameters to be taken into account by the clinician. The pharmaceutical composition of this invention can be lyophilized for storage and reconstituted in a suitable carrier prior to use. When prepared as lyophilization or liquid, physiologically acceptable carrier, excipient, stabilizer need to be added into the pharmaceutical composition of the invention (Remington's Pharmaceutical Sciences 22nd edition, Ed. Allen, Loyd V, Jr. (2012). The dosage and concentration of the carrier, excipient and stabilizer should be safe to the subject (human, mice and other mammals), including buffers such as phosphate, citrate, and other organic acid; antioxidant such as vitamin C, small polypeptide, protein such as serum albumin, gelatin or immunoglobulin; hydrophilic polymer such as PVP, amino acid such as amino acetate, glutamate, asparagine, arginine, lysine; glycose, disaccharide, and other carbohydrate such as glucose, mannose or dextrin, chelate agent such as EDTA, sugar alcohols such as mannitol, sorbitol; counterions such as Na+, and/or surfactant such as TWEEN™, PLURONICS™ or PEG and the like.

### DETAILED DESCRIPTION

In a first aspect of the invention, a antigen-binding protein-containing binding agent specifically binding the human Na+-taurocholate co-transporting polypeptide (NTCP) is disclosed, wherein said NTCP-specific antigen-binding protein-containing binding agent is an allosteric NTCP transport inhibitor.

The solute carrier family SLC10, or “sodium bile acid cotransporter family° counts 7 members (SLC10A1-SLC10A7), of which SLC10A1 was characterized as the Na⁺/taurocholate cotransporting polypeptide providing for a hepatic bile acid transporter (NTCP, gene symbol SLC10A1). NTCP mediates sodium (Na⁺)-coupled uptake of taurocholic acid (TC) and other bile acids (BA) in the liver (also referred to herein as bile salts or sodium ion/bile acids), making the transporter essential for maintaining the enterohepatic circulation of BAs. Besides, NTCP has also been identified as the high-affinity hepatic entry receptor for the hepatitis B and D viruses. HBV/HDV viruses bind to NTCP with their 2-48 N-terminal amino acids of the myristoylated preS1 domain (also called myr-preS1 peptide) of the large envelope protein which triggers cellular entry, thereby positioning NTCP as a potential target for the development of HBV and/or HDV entry inhibitors, which is currently a field that is mainly based on small molecules with oral bioavailability, or peptides mimicking the mur-PreS1 peptide). The substrate binding sites on NTCP, for sodium-coupled BAs (taurocholic acid (TC), Taurolithocholic acid (TLC), dehydroepiandrosterone sulfate (DHEAS)) and the binding site for myr-PreS1 peptide binding to NTCP are known to directly interfere with each other, since BAs can block myr-preS1 peptide binding to NTCP and myr-preS1 peptide binding to NTCP inhibits BA transport. So, the myr-preS1 lipopeptide showed equipotent inhibition of all substrates (TC, TLC, and DHEAS) of NTCP, suggesting that this peptide completely blocks the access of any substrate to its respective binding site (Grosser et al. (2021) Front. Mol. Biosci. 8:689757). So the substrate binding site and myr-PreS1 binding site on NTCP are at least overlapping, and compete for binding.

The NTCP-specific antigen-binding protein-containing binding agents disclosed herein were structurally analyzed in complex with NTCP and shown not to compete with the myr-PreS1 binding site, does not putting steric hindrance on the PreS1 binding, since for the ‘open pore conformation state’ binder Nb_{NTCP}91/NTCP_{EM} complex it was shown that binding of myr-PreS1 was still obtained, while for the ‘inward-facing conformation state’ binder Nb_{NTCP}87/NTCP_{EM} complex it was shown that binding of myr-PreS1 was prohibited, not by competition or sterical hindrance, but due to the induced conformational state stabilized by the bound Nb, thereby interfering with the NTCP binding of the peptide.

As disclosed herein, the antigen-binding protein-containing binding agents of the present invention, when bound to NTCP, provide for inhibition of Bile salt transport activity (also see example section), wherein the inhibition means that the transport activity of the Nb:NTCP/NTCP complex is reduced with at least 10% as compared to the functional NTCP (without the presence of Nb or in the presence of a negative control Nb), or is reduced with at least 20%, at least 30%, at least 40%, at least 45%, at least 50%, at least 55%, at least 60%, at least 70%, at least 80%, at least 90%, at least 95%, or completely abolished (undetectable) as compared to the wild type NTCP control. The skilled artisan is aware of methods known in the art usable to test the transport activity of NTCP, such as for instance the Fluorescent substrate-analog transport assay as described herein (see Methods in Example section), providing that transport activity of NTCP is inhibited in the presence of the binding agent if the Na⁺-induced fluorescent-substrate uptake by cells expressing NTCP_{EM} has a half-maximal inhibitory concentrations (IC₅₀) in the nanomolar range, or more specifically provides for an IC₅₀ of 900 nM or lower, of 800 nM or lower, of 700 nM or lower, of 600 nM or lower, of 500 nM or lower, of 400 nM or lower, of 300 nM or lower, of 250 nM or lower, of 200 nM or lower, of 180 nM or lower, of 150 nM or lower, of 100 nM or lower, of 80 nM or lower, of 50 nM or lower, of 30 nM or lower, of 10 nM or lower, of 1 nM or lower, or of 0.1 nM. Alternatively, the inhibition of BS or BA transport activity is determined using the ATP-dependent transport of labeled bile salts as determined by a rapid filtration assay as described in Gerloff et al (J Biol Chem. 1998; 273:10046-10050), or among others, an in vitro assay developed for determining inhibition of the bile salt export using hepatocyte suspension as described in Zhang, et al. (2016, Chemico-Biological Interactions, Vol 255, p. 45-54), or a bile acid uptake assay as described in [87].

The term “allosteric inhibition°, “allosteric activity° or “allosteric regulation°, as used herein, refers to binding of said agent at an allosteric or regulatory site, which is a site different from the substrate binding active site or catalytic site of the protein, so different to the binding site of orthosteric binders. Thus the binding agent defined herein as an allosteric inhibitor is in its NTCP-bound state bound to a conformational epitope of NTCP (the open pore or inward-facing state), thereby stabilizing this BTCP protein conformation, which results in a protein fold that inhibits hepatic bile acid transport. Thus the allosteric inhibition by the NTCP-bound binding agent is thus nor a result of steric hindrance of the substrate binding, neither a result of competition for substrate binding. Such substrate binding sites of NTCP are known in the art and comprise for instance the binding sites of Bile acids (TC, TLC, and DHEAS) and salts thereof. Moreover, in its function of HBV/HDV entry receptor, the active site or substrate binding site may be considered as the myr-PreS1 peptide binding site. The allosteric binding agent disclosed herein may for instance affect or modulate the NTCP transport and/or viral entry activity, through the induction of a conformational change of the NTCP protein upon binding. So in a specific embodiment, the NTCP-binding agent is an allosteric inhibitor by binding to an allosteric site which alters the protein conformation relevant to the transport activity of NTCP which consequently changes the transport activity. Indeed, it is generally considered that alternating conformations open to in- and outside of the membrane involve bile salt transport activity, whereas the NTCP-specific binding agents disclosed herein lock or stabilize one specific conformation, either an inward-facing or inside-open conformation or an open pore or outside-open conformation. Thus bile salt substrates are no longer able to cross the membrane through the NTCP transport mechanism, and the binding agents provide for allosteric inhibition of human NTCP.

Another embodiment relates to the NTCP-specific binding agent which is an allosteric inhibitor of BS transport, wherein said agent comprises a NTCP-specific antigen-binding protein which is an antibody, an antibody mimetic, a single domain antibody, an immunoglobulin single variable domain (ISVD), a VHH, as disclosed and defined herein and as known to the skilled person.

Antibody mimetics are organic compounds that, like antibodies, can specifically bind antigens, but that are not structurally related to antibodies. They are usually artificial peptides or proteins with a molar mass of about 3 to 20 kDa. Examples of antibodymimetics include but are not limited to: Affibodies, Affilins, Affimers, Affitins, Alphabodies, Anticalins, Avimers, DARPins, Fynomers, Gastrobodies, Kunitz domain peptides, Monobodies, Optimers, and Obodies among others.

A further specific embodiment relates to the NTCP-specific antigen-binding protein-containing binding agent which is an allosteric BS transport inhibitor and wherein the antigen-binding protein comprises an immunoglobulin single variable domain (ISVD), as defined herein, or more specifically comprises a VHH or a Nanobody, wherein said ISVD, VHH or Nanobody upon binding to a conformational epitope on the extracellular portion of NTCP stabilizes a conformational state, preferably an ‘inward-facing’ or ‘open pore’ conformational state, as defined herein.

The binding agents according to the current invention are in another aspect structurally defined as polypeptidic binding agents (i.e. binding agents comprising a peptidic, polypeptidic or proteic moiety, or binding agents comprising a peptide, polypeptide, protein or protein domain) or polypeptide binding agents (i.e. binding agents being peptides, polypeptides or proteins). In a specific embodiment, the polypeptidic binding agent of the present invention is not a linear peptide. More in particular, the binding agents according to the current invention can be structurally defined as polypeptidic or polypeptide binding agents comprising an antigen-binding domain, more specifically an ISVD, more specifically at least one ISVD comprising a complementarity determining region (CDR) as comprised in any of the immunoglobulin single variable domains (ISVDs) defined hereinafter. More in particular, the binding agents according to the current invention can in one embodiment be structurally defined as polypeptidic or polypeptide binding agents comprising at least CDR3 as comprised in an immunoglobulin single variable domains (ISVDs) as defined hereinafter. In another embodiment, the binding agents according to the current invention can be structurally defined as polypeptidic or polypeptide binding agents comprising at least two of CDR1, CDR2 and CDR3 (e.g. CDR1 and CDR3, CDR2 and CDR3, CDR1 and CDR2), or all three of CDR1, CDR2 and CDR3, as comprised in an immunoglobulin single variable domains (ISVDs) as defined hereinafter.

In a particular embodiment, the NTCP-specific binding agent as described herein, comprises one or more antigen-binding proteins containing ISVDs comprising the complementarity determining regions (CDRs) as present in any of the following Nbs which are provided herein as allosteric BS (or bile acid (BA), as interchangeably used herein) transport inhibitors, namely the Nbs of Family 21, comprising Nb_{NTCP}87 (SEQ ID NO: 5) and Nb_{NTCP}88 (SEQ ID NO: 6), the Nbs of family 05, comprising Nb_{NTCP}53 (SEQ ID NO: 14), or the Nbs of family 15, comprising Nb_{NTCP}66-71 (SEQ ID NO: 19-24), wherein the CDRs are annotated according to Kabat, MacCallum, IMGT, AbM, or Chothia.

In a particular embodiment the NTCP-specific binding agent as described herein, comprises one or more ISVDs comprising the complementarity determining regions (CDRs) as present in Nb_{NTCP}87 (SEQ ID NO: 5), specifically with CDR1 comprising SEQ ID NO:40, CDR2 comprising SEQ ID NO: 41, and CDR3 comprising SEQ ID NO: 42, wherein CDR sequences were defined according to Kabat annotation.

In an alternative embodiment, the NTCP-specific binding agents described herein comprise at least one ISVD comprising a sequence corresponding to the sequence of Nbs of Family 21, comprising Nb_{NTCP}87 (SEQ ID NO: 5) and Nb_{NTCP}88 (SEQ ID NO: 6), the Nbs of family 05, comprising Nb_{NTCP}53 (SEQ ID NO: 14), or the Nbs of family 15, comprising Nb_{NTCP}66-71 (SEQ ID NO: 19-24), or a functional variant of any one thereof with at least 90%, at least 95%, at least 97%, or at least 98%, or at least 99% identity over the full length of the ISVD sequence wherein the non-identical amino acids are located in one or more Framework residues, or a humanized variant of any one thereof, as described herein. In particular, such non-identity or variability, is limited to non-identity or variability in FR amino acid residues. In particular, such non-identity or variability may be introduced to obtain a humanized variant of an ISVD defined by or set forth in any of to an amino acid sequence selected from the group of SEQ ID NOs: 5, 6, 14, 19-24. In particular, such humanized variant is a functional orthologue of the original ISVD, wherein the functionality is defined as described herein.

Alternatively, single mutants of Nb_{NTCP}87 (SEQ ID NO: 5) with retained functionality (i.e. functional variants) are provided herein, based on the structural information of the Nb_{NTCP}87/NTCP complex disclosed herein, providing for NTCP-specific binding agents that are allosteric BA transport inhibitors as described herein, wherein CDR1 comprises SEQ ID NO:40, CDR2 comprises SEQ ID NO: 41, 46 or 47, and CDR3 comprises SEQ ID NO: 42, 48 or 49. Alternatively, a binding agent comprising an ISVD with any combination of said single mutant substitutions of the CDRs of Nb_{NTCP}87 is envisaged herein.

More specifically, the NTCP-specific binding agent are envisaged herein comprising an ISVD comprising a sequence corresponding to the sequence of such single mutant Nbs of Nb_{NTCP}87, as defined in SEQ ID NOs: 25-29, or a functional variant of any one thereof with at least 90%, at least 95%, at least 97%, or at least 98%, or at least 99% identity over the full length of the ISVD sequence wherein the non-identical amino acids are located in one or more Framework residues, or a humanized variant of any one thereof, as described herein. In particular, such non-identity or variability may be introduced to obtain a humanized variant of an ISVD defined by or set forth in any of to an amino acid sequence selected from the group of SEQ ID NOs: 25-29. In particular, such humanized variant is a functional orthologue of the original ISVD, wherein the functionality is defined as described herein.

Alternatively, said binding agent comprising an ISVD with a sequence containing a combination of any of said single mutant substitutions of Nb_{NTCP}87 is envisaged herein, more specifically the sequence of Nb_{NTCP}87 (SEQ ID NO:5), wherein the amino acid at position 55 of SEQ ID NO:5 is an S, Q or E, wherein the amino acid at position 30 of SEQ ID NO:5 is an A or Q, wherein the amino acid at position 104 of SEQ ID NO:5 is an S or G, and/or wherein the amino acid at position 111 of SEQ ID NO:5 is an S or R.

In a particular embodiment, the NTCP-specific binding agent as described herein, comprises one or more ISVDs comprising the complementarity determining regions (CDRs) as present in any of the following Nbs which are provided herein as allosteric BS transport inhibitors, namely the Nbs of Family 23, comprising Nb_{NTCP}91 (SEQ ID NO: 7), the Nbs of family 18, comprising Nb_{NTCP}79 (SEQ ID NO: 8), Nb_{NTCP}80 (SEQ ID NO: 9), Nb_{NTCP}81 (SEQ ID NO: 10), or the Nbs of family 19, comprising Nb_{NTCP}82 (SEQ ID NO: 11), Nb_{NTCP}83 (SEQ ID NO: 12), Nb_{NTCP}84 (SEQ ID NO: 13), or the Nbs of family 17, comprising Nb_{NTCP}74-77 (SEQ ID NO: 15-18), wherein the CDRs are annotated according to Kabat, MacCallum, IMGT, AbM, or Chothia.

In a particular embodiment the NTCP-specific binding agent as described herein, comprises one or more ISVDs comprising the complementarity determining regions (CDRs) as present in Nb_{NTCP}91 (SEQ ID NO: 7), specifically with CDR1 comprising SEQ ID NO:43, CDR2 comprising SEQ ID NO: 44, and CDR3 comprising SEQ ID NO: 45, wherein CDR sequences were defined according to Kabat annotation.

In an alternative embodiment, the NTCP-specific binding agents described herein comprise at least one ISVD comprising a sequence corresponding to the sequence of Nbs of Family 23, comprising Nb_{NTCP}91 (SEQ ID NO: 7), the Nbs of family 18, comprising Nb_{NTCP}79 (SEQ ID NO: 8), Nb_{NTCP}80 (SEQ ID NO: 9), Nb_{NTCP}81 (SEQ ID NO: 10), or the Nbs of family 19, comprising Nb_{NTCP}82 (SEQ ID NO: 11), Nb_{NTCP}83 (SEQ ID NO: 12), Nb_{NTCP}84 (SEQ ID NO: 13), or the Nbs of family 17, comprising Nb_{NTCP}74-77 (SEQ ID NO: 15-18), or a functional variant of any one thereof with at least 90%, at least 95%, at least 97%, or at least 98%, or at least 99% identity over the full length of the ISVD sequence wherein the non-identical amino acids are located in one or more Framework residues, or a humanized variant of any one thereof, as described herein. In particular, such non-identity or variability may be introduced to obtain a humanized variant of an ISVD defined by or set forth in any of to an amino acid sequence selected from the group of SEQ ID NOs: 7-13, or 15-18. In particular, such humanized variant is a functional orthologue of the original ISVD, wherein the functionality is defined as described herein.

Alternatively, single mutants of Nb_{NTCP}91 (SEQ ID NO: 7) with retained functionality (i.e. functional variants) are provided herein, based on the structural information of the Nb_{NTCP}91/NTCP complex disclosed herein, providing for NTCP-specific binding agents that are allosteric inhibitors as described herein, wherein CDR1 comprises SEQ ID NO:43, CDR2 comprises SEQ ID NO: 44 or 50, and CDR3 comprises SEQ ID NO: 45 or 51. Alternatively, a binding agent comprising an ISVD with any combination of said single mutant substitutions of the CDRs of Nb_{NTCP}91 is envisaged herein.

More specifically, the NTCP-specific binding agent are envisaged herein comprising an ISVD comprising a sequence corresponding to the sequence of such single mutant Nbs of Nb_{NTCP}91, as defined in SEQ ID NOs: 30-37, or a functional variant of any one thereof with at least 90%, at least 95%, at least 97%, or at least 98%, or at least 99% identity over the full length of the ISVD sequence wherein the non-identical amino acids are located in one or more Framework residues, or a humanized variant of any one thereof, as described herein. In particular, such non-identity or variability may be introduced to obtain a humanized variant of an ISVD defined by or set forth in any of to an amino acid sequence selected from the group of SEQ ID NOs: 30-37. In particular, such humanized variant is a functional orthologue of the original ISVD, wherein the functionality is defined as described herein.

Alternatively, said binding agent comprising an ISVD with a sequence containing a combination of any of said single mutant substitutions of Nb_{NTCP}91 is envisaged herein, more specifically the sequence of Nb_{NTCP}91 (SEQ ID NO: 7), wherein the amino acid at position 27 of SEQ ID NO: 7 is an N, D, Q or E, wherein the amino acid at position 29 of SEQ ID NO:7 is an R, N, G or S, wherein the amino acid at position 53 of SEQ ID NO:7 is an N or D, and/or wherein the amino acid at position 103 of SEQ ID NO:7 is an L or N.

In a further specific embodiment, said binding agents or binding domains as defined by the ISVDs comprising the CDRS as listed above, are provided by a functional variant thereof, characterized in that said variant still provides for the same or very similar binding and inhibitory properties, and/or a humanized variant thereof, as described herein, and as exemplified by Nb87 humanized variants 1˜4 (SEQ ID No: 70-73, and Nb91 humanized variants 1-2 (SEQ ID NOs: 74-75), wherein one or more amino acids in the framework regions are substituted selected from the residues corresponding to (according to Kabat numbering) any one or more of Q1, A14, V59, A63, S77, D82a, K83, Q108 or Q1, E13, A14, I76, K83 or Q108, respectively.

A further specific embodiment relates to said NTCP-specific binding agents which comprise an ISVD that is a functional or humanized variant of any one of SEQ ID NOs: 5-37 with at least 90% identity wherein the CDRs are identical, and wherein said 90% identity is calculated for instance for FR1 of the variant being 90% identical to the entire length of FR1 of the original VHH sequence as depicted in any of SEQ ID NOs: 5-37, and FR2 of the variant being 90% identical to the entire length of FR2 of the original VHH sequence as depicted in any of SEQ ID NOs: 5-37, and FR3 of the variant being 90% identical to the entire length of FR3 of the original VHH sequence as depicted in any of SEQ ID NOs: 5-37, and FR4 of the variant being 90% identical to the entire length of FR4 of the original VHH sequence as depicted in any of SEQ ID NOs: 5-37, and/or alternatively, the CDRs being identical, but the full length sequence being at least 90% identical to the original VHH sequence of SEQ ID NOs: 5-37.

In a further aspect, the polypeptidic or polypeptide binding agents according to the current invention may comprise one or more framework regions (FRs) as comprised in any of the ISVDs defined hereinabove.

Another aspect of the invention relates the NTCP-specific antigen-binding protein-containing binding agent which is an allosteric BS transport inhibitor, which is a multivalent or multispecific agent, as defined herein. In a specific embodiment the multivalent or multispecific moieties of said binding agent or directly linked or fused by a short spacer or linker. Alternatively said multivalent or multispecific moieties of said binding agent are present in the format of an Fc fusion or an antibody, or any other chimeric format as known to the skilled person and/or as described herein. So, another embodiment relates to said polypeptidic or polypeptide binding agents that are comprising one or more ISVDs (or variants or humanized forms thereof as described herein) specifically binding human NTCP, wherein the at least one or more ISVD (or variant or humanized form thereof as described herein) are linked to another ISVD, or another moiety by direct linking or by fusion via a spacer or linker, such as a peptide linker. In another embodiment, said polypeptidic or polypeptide binding agents that are comprising one or more ISVDs (or variants or humanized forms thereof as described herein) specifically binding human NTCP, the at least one or more ISVD (or variant or humanized form thereof as described herein) is bound or fused to an Fc domain, which is defined herein as the fragment crystallizable region (Fc region) of an antibody, which is the tail region known to interact with cell surface receptors called Fc receptors and some proteins of the complement system. Said Fc domain is composed of two identical protein fragments, derived from the second and third constant domains of the antibody's two heavy chains. All conventional antibodies comprise an Fc domain, hence, the Fc domain fusion may comprise an Fc domain derived from or as a variant of the IgG, IgA and IgD antibody Fc regions, even more specifically an IgG1, IgG2 or IgG4. The hinge region of IgG2, may be replaced by the hinge of human IgG1 to generate ISVD fusion constructs, and vice versa. Additional linkers that are used to fuse a herein identified ISVD to the IgG1 and IgG2 Fc domains comprise (G₄S)₂₋₃. In addition, Fc variants with known half-life extension may be used such as the M257Y/S259T/T261E (also known as YTE) or the LS variant (M428L combined with N434S). These mutations increase the binding of the Fc domain of a conventional antibody to the neonatal receptor (FcRn).

In some embodiments, the Fc region is engineering to create “knobs” and “holes” which facilitate heterodimer formation of two different Fc-containing polypeptide chains when co-expressed in a cell (U.S. Pat. No. 7,695,963). The Fc region may be altered to use electrostatic steering to encourage heterodimer formation while discouraging homodimer formation of two different Fc-containing polypeptide when co-expressed in a cell (WO 09/089,004).

In yet another aspect, the invention provides nucleic acid molecules such as isolated nucleic acids, (isolated) chimeric gene constructs, expression cassettes, recombinant vectors (such as expression or cloning vectors) comprising a nucleotide sequence, such a coding sequence, that is encoding the polypeptide binding agent or NTCP-specific binding agent as identified herein.

One further aspect of the invention provides for a host cell comprising the binding agent(s), such as an ISVD, as described herein. The host cell may therefore comprise the nucleic acid molecule encoding said polypeptide binding agent or NTCP-specific binding agent. The host cell may also be transfected with the binding agent or nucleic acid molecule encoding the binding agent as disclosed herein. Host cells can be either prokaryotic or eukaryotic. The host cell may also be a recombinant host cell, which involves a cell which has been genetically modified to contain an isolated DNA molecule, nucleic acid molecule encoding the polypeptide binding agent of the invention. Representative host cells that may be used to produce said ISVDs, but are not limited to, bacterial cells, yeast cells, plant cells and animal cells. Bacterial host cells suitable for production of the binding agents of the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. Animal host cells suitable for use with the invention include insect cells and mammalian cells (most particularly derived from Chinese hamster (e.g. CHO), and human cell lines, such as HeLa). Exemplary insect cell lines include, but are not limited to, Sf9 cells, baculovirus-insect cell systems (e.g. review Jarvis, Virology Volume 310, Issue 1, 25 May 2003, Pages 1-7). Alternatively, the host cells may also be transgenic animals or plants.

In a further aspect of the invention the NTCP-specific binding agent disclosed herein comprises a label or tag, more specifically an ISVD of the binding agent is labeled or tagged, or has a detectable moiety fused to it, bound to it, coupled to it, linked to it, complexed to it, or chelated to it. A “label° or “detectable moiety in general refers to a molecule or moiety that emits a signal or is capable of emitting a signal upon adequate stimulation, or to a moiety that is capable of being detected through binding or interaction with a further molecule (e.g. a tag, such as an affinity tag, that is specifically recognized by a labelled antibody), or is detectable by any means (preferably by a non-invasive means, if detection is in vivo/inside the human body). Furthermore, the detectable moiety may allow for computerized composition of an image, as such the detectable moiety may be called an imaging agent. Detectable moieties include fluorescence emitters, phosphorescence emitters, positron emitters, radioemitters, etc., but are not limited to emitters as such moieties also include enzymes (capable of measurably converting a substrate) and molecular tags. Examples of fluorescence emitters include cyanine dyes (e.g. Cy5, Cy5.5, Cy7, Cy7.5), FITC, TRITC, coumarin, indolenine-based dyes, benzoindolenine-based dyes, phenoxazines, BODIPY dyes, rhodamines, Si-rhodamines, Alexa dyes, and derivatives of any thereof. Further examples of labels, tags or detectable moieties, as used interchangeably herein include but are not limited to affinity tags, such as chitin binding protein (CBP), maltose binding protein (MBP), glutathione-S-transferase (GST), poly(His) (e.g., 6× His or His6), biotin or streptavidin, such as Strep-tag®, Strep-tag II® and Twin-Strep-tag®; solubilizing tags, such as thioredoxin (TRX), poly (NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase).

Binding agents as described herein comprising a detectable moiety may for example be used for in vitro, in vivo or in situ assays (including immunoassays known per se such as ELISA, RIA, EIA and other “sandwich assays”, etc.) as well as in vivo imaging purposes, depending on the choice of the specific label.

In another aspect of the invention the NTCP-specific binding agent described herein comprises an ISVD that is conjugated to a further functional moiety, wherein the term ‘functional moiety’ refers to a molecule or component which performs an additional function for the binding agents when used for a specific purpose. Said purpose may for instance but non-limiting include the purpose of therapeutic use, diagnostic use, the use as vehicle in targeted-delivery, the use in drug discovery or screening assays, the use in structural analysis, the use in gene therapy, among others. So the functional moiety conjugated to the NTCP-specific ISVD of the binding agent may for instance comprise a therapeutic moiety, such as a biological or liver-target-specific drug, a half-life extension, a small-molecule compound, an enzyme, an antibody, a genome-editing component, such as a nuclease, a nucleic acid molecule, or a nanoparticle such as a liposome.

In a particular embodiment, the binding agent is part of a liposomal composition, and may be present as a conjugate to said liposome for liver-specific targeted delivery of said liposomal composition, wherein said liposome may contain additional therapeutic moieties. A further specific embodiment relates to a surface-coated nanoparticle with the NTCP-specific ISVD as described herein, said nanoparticle having biodegradable polymer chains which may further comprise an agent encapsulated within said nanoparticle or liposome. In another embodiment the NTCP-specific binding agent described herein is conjugated to a nanoparticle which encapsulates a further antiviral agent for HBV/HDV treatment, as to provide for a combinatorial therapeutic approach using a single composition.

Said liposomal or nanoparticle composition may be provided in a freeze-dried state in the presence of a lyoprotectant, as a capsule or a tablet. Said composition or nanoparticle conjugate as described herein may be used in a method of treatment of a liver disorder and/or HBV/HDV chronic infection of an animal or human body.

A further aspect of the invention relates to a pharmaceutical composition comprising the NTCP-specific binding agent as described herein, the multivalent or multispecific binding agent as described herein, the liposomal composition or otherwise functionally conjugated binding agent as described herein, the nucleic acid molecule or vector as described herein, and/or optionally a further therapeutic agent, a carrier, excipient or diluent, as defined herein. A further embodiment relates to medicaments or pharmaceutical compositions comprising the binding agent(s) and/or nucleic acid encoding it, and/or a recombinant vector comprising the nucleic acid, as described herein. In particular, a pharmaceutical composition is a pharmaceutically acceptable composition; such compositions are in a particular embodiment further comprising a (pharmaceutically) suitable or acceptable carrier, diluent, stabilizer, etc.

Alternatively, use of said binding agent or nucleic acid encoding it as described herein, or use of a pharmaceutical composition comprising a binding agent, nucleic acid encoding it, and/or a recombinant vector comprising such nucleic acid, as described herein, in the manufacture of a medicine or medicament is envisaged. In particular, the composition, binding agent or nucleic acid encoding it as described herein, or the medicament or pharmaceutical composition comprising a binding agent, nucleic acid encoding it, and/or a recombinant vector comprising such nucleic acid, as described herein, is for use in treating a subject with hepatitis B/D viral infection, more specifically for use in treatment of chronic HBV/HDV infection. Indeed, the binding agents comprising Nb_{NTCP} molecules as described herein can act in several fronts regarding HBV and HDV chronic infections. First, as cell entry inhibitors that preclude preS1 binding to the surface of the hepatocyte^{2,3}. Though, complete functional cure of HBV and HDV chronic infections may require combination therapy targeting in addition intracellular steps of the viral life cycles particularly viral DNA (known as cccDNA), and the protein that controls its expression, so called HBx⁸⁰⁻⁸². Second, the binding agents comprising Nb_{NTCP} molecules described herein derivatized with therapeutic small-compound molecules, enzymes, antibodies, genome-editing components, RNAs, DNAs, or nanoparticles (e.g. liposomes) can aid targeting these materials into the hepatocyte. Finally, since the binding agents comprising Nb_{NTCP} molecules as described herein stabilize different conformational states of the NTCP transport cycle, screening of small-compound molecules that preferentially bind to particular NTCP conformational states, e.g. molecules that stabilize inward-facing states among test compound libraries⁸³ is envisaged herein.

In a further particular embodiment, the composition, binding agent or nucleic acid encoding it as described herein, or the medicament or pharmaceutical composition comprising a binding agent, nucleic acid encoding it, and/or a recombinant vector comprising such nucleic acid, as described herein, is used in treating a subject with a liver disease or disorder. More specifically, liver disorders and diseases referred to herein include liver fibrosis⁸⁵ which leads to cirrhosis and hepatocellular carcinoma, since liver stellate cells, which are the ones producing fibrosis, and express some levels of NTCP in their surface during differentiation⁸⁶; hepatitis, autoimmune hepatitis, non-alcoholic fatty liver disease (NAFLD), (hereditary) hemochromatosis, Wilson's disease, tropical diseases, such as malaria, schistosomiasis, leishmaniasis; liver tumors, liver metastases (e.g. colorectal carcinoma), and metabolic diseases, such as obesity, and dyslipidemias, particularly regarding lowering-cholesterol therapies^{16,17} metabolic syndrome, non-alcoholic steatohepatitis; atherosclerosis, an NRLP3 inflammasome-associated disease, such as type-2 diabetes, gout, Alzheimer's disease and NASH; alcoholic steatohepatitis (ASH), and genetic liver disorders.

A further aspect of the invention relates to methods for treating a subject suffering from/having/that has contracted an infection with HBV/HDV, or a liver disease, the methods comprising administering the binding agent or composition comprising said binding agent(s) or nucleic acid encoding it as described herein to the subject, or comprising administering a medicament or pharmaceutical composition comprising a binding agent or nucleic acid encoding it as described herein to the subject.

Furthermore, in particular to the above medical aspects, a nucleic acid encoding a binding agent as described herein can be used in e.g. gene therapy setting for delivery to hepatocytes. Hepatic gene therapy can be used to treat genetic disorders, liver metabolic diseases and hepatocellular carcinoma, as well as viral hepatitis. Moreover, hepatocytes produce important proteins for serum, and gene therapy can restore the protein production capabilities of hepatocytes⁸⁴. Therefore, the binding agents disclosed herein comprising Nb_{NTCP} molecules linked to nanoparticles carrying RNAs or DNAs may be envisaged to aid in entry into the hepatocytes.

Moreover, since hepatocytes constitute ˜80% of liver parenchyma, and constitute the main cell type that expresses NTCP, another aspect relates to the use of the NTCP-specific binding agents or pharmaceutical compositions disclosed herein for hepatocyte specific targeting and use as a drug delivery tool or as a nanomedicine or nanomaterial-based drug delivery system. Several important liver diseases, in addition to viral hepatitis, require targeted delivery of drugs to hepatocytes, including alcohol-induced steatohepatitis, steatohepatitis, non-alcohol-induced Wilson's disease, hemochromatosis, alpha-1 antitrypsin deficiency, among other metabolic disorders⁸⁴. Binding agents comprising Nb_{NTCP} molecules derivatized with therapeutics against the above mention diseases are thus envisaged for use as vehicles to achieve liver tropism and targeted drug delivery. Alternatively, said NTCP-specific binding agents or pharmaceutical composition disclosed herein is useful for targeted enzyme delivery for detoxification and local prodrug conversion in the liver.

In another alternative aspect of the invention, any of the compositions or binding agents described herein, optionally with a label, or any of the nucleic acid molecules encoding said agent, or any of the pharmaceutical compositions, or vectors as described herein may as well be used as a diagnostic. Diagnostic methods are known to the skilled person and may involve biological samples from a subject. Also in vitro methods may be in scope for detection of using the binding agents as described herein. Finally, the composition or binding agents as described herein, optionally labelled, may also be suitable for use in in vivo, in situ or ex vivo liver imaging.

A further aspect of the invention relates to kits comprising a composition or binding agent or nucleic acid encoding it as described herein, or a pharmaceutical composition comprising a binding agent or nucleic acid encoding it as described herein.

Such kits comprise pharmaceutical kits or medicament kits which are comprising a container or vial (any suitable container or vial, such as a pharmaceutically acceptable container or vial) comprising an amount of binding agent or nucleic acid encoding it as described herein, and further comprising e.g. a kit insert such as a medical leaflet or package leaflet comprising information on e.g. intended indications and potential side-effects. Pharmaceutical kits or medicament kits may further comprise e.g. a syringe for administering the binding agent or nucleic acid encoding it as described herein to a subject.

In another aspect, a screening method to identify a conformation-selective compound specifically binding human NTCP is disclosed, said method comprising the steps of:
a) Combining the NTCP-conformation-selective binding agent as disclosed herein with a sample comprising NTCP, or providing the NTCP complex with said binding agent, and
b) Adding a test compound to the sample of a), under suitable conditions to allow binding to NTCP, and
c) Identify said compound as a conformation-selective compound when the test compound specifically binds to human NTCP in complex with the binding agent, and wherein the binding agent is in complex with human NTCP which is present in an inward-facing or an open pore conformational state.

In one embodiment, said sample with NTCP in step a. may be a biological sample, a protein sample, a membranous fraction, a cell expressing NTCP on the surface, or any other sample containing a portion of NTCP that is exposed at the outside of a membrane, preferably an in vitro sample.

The term “compound° or “test compound° or “candidate compound° or “drug candidate compound° as used herein describes any molecule, either naturally occurring or synthetic that may be tested in an assay, such as a screening assay or drug discovery assay, or specifically in the method for identifying a conformation-selective compound specifically binding human NTCP. As such, these compounds comprise organic and inorganic compounds. The compounds may be small molecules, chemicals, peptides, antibodies or ISVDs or active antibody fragments. Compounds of the present invention include both those designed or identified using a screening method of the invention (as described herein for instance) and those which are capable of binding NTCP in a conformation-selective manner as defined above.

Said compounds may be produced using a screening method based on a screening assay making use of the binding agents disclosed herein or based on use of the atomic coordinates corresponding to the 3D structure of NTCP_{EM}/Nb_{NTCP} complexes as presented herein (see below). The candidate compounds and/or compounds identified or designed using a method and or the binding agents of the present invention or derivatives thereof may be any suitable compound, synthetic or naturally occurring, preferably synthetic. In one embodiment, a synthetic compound selected or designed by the methods of the invention preferably has a molecular weight equal to or less than about 5000, 4000, 3000, 2000, 1000 or more preferably less than about 500 daltons. A compound of the present invention is preferably soluble under physiological conditions. The compounds may encompass numerous chemical classes, though typically they are organic molecules, preferably small organic compounds having a molecular weight of more than 50 and less than about 2,500 daltons, preferably less than 1,500, more preferably less than 1,000 and yet more preferably less than 500. Such compounds can comprise functional groups necessary for structural interaction with proteins, particularly hydrogen bonding, and typically include at least an amine, carbonyl, hydroxyl or carboxyl group, preferably at least two of the functional chemical groups. The compound may comprise cyclical carbon or heterocyclic structures and/or aromatic or polyaromatic structures substituted with one or more of the above functional groups. Compounds can also comprise biomolecules including peptides, saccharides, fatty acids, steroids, purines, pyrimidines, derivatives, structural analogues, or combinations thereof. Compounds may include, for example: (1) peptides such as soluble peptides, including Ig-tailed fusion peptides and members of random peptide libraries and combinatorial chemistry-derived molecular libraries made of D- and/or L-configuration amino acids; (2) phosphopeptides (e.g. members of random and partially degenerate, directed phosphopeptide libraries, (3) antibodies (e.g., polyclonal, monoclonal, humanized, anti-idiotypic, chimeric, and single chain antibodies, nanobodies as well as Fab, (Fab)₂, Fab expression library and epitope-binding fragments of antibodies); (4) non-immunoglobulin binding proteins such as but not restricted to avimers, DARPins and lipocalins; (5) nucleic acid-based aptamers; and (6) small organic and inorganic molecules.

Synthetic compound libraries are commercially available from, for example, Maybridge Chemical Co. (Tintagel, Cornwall, UK), AMRI (Budapest, Hungary) and ChemDiv (San Diego, Calif.), Specs (Delft, The Netherlands), ZINC15 (Univ. of California). In addition, numerous means are available for random and directed synthesis of a wide variety of organic compounds and biomolecules, including expression of randomized oligonucleotides. Alternatively, libraries of natural compounds in the form of bacterial, fungal, plant and animal extracts can be readily produced. In addition, natural or synthetic compound libraries and compounds can be readily modified through conventional chemical, physical and biochemical means and may be used to produce combinatorial libraries. In another approach, previously identified pharmacological agents can be subjected to directed or random chemical modifications, such as acylation, alkylation, esterification, amidification, and the analogues can be screened for NTCP conformation-selective binding. In addition, numerous methods of producing combinatorial libraries are known in the art, including those involving biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide or peptide libraries, while the other four approaches are applicable to polypeptide, peptide, nonpeptide oligomer, or small molecule libraries of compounds. Further, compounds identified or designed using the methods of the invention can be a peptide or a mimetic thereof. The isolated peptides or mimetics of the invention may be conformationally constrained molecules or alternatively molecules which are not conformationally constrained such as, for example, non-constrained peptide sequences. The term “conformationally constrained molecules” means conformationally constrained peptides and conformationally constrained peptide analogues and derivatives. In addition, the amino acids may be replaced with a variety of uncoded or modified amino acids such as the corresponding D-amino acid or N-methyl amino acid. Other modifications include substitution of hydroxyl, thiol, amino and carboxyl functional groups with chemically similar groups. With regard to peptides and mimetics thereof, still other examples of other unnatural amino acids or chemical amino acid analogues/derivatives can be introduced as a substitution or addition. Also, a peptidomimetic may be used.

The ability of a candidate compound to increase or decrease the binding to the NTCP/Nb_{NTCP} complex as disclosed herein, can be assessed by any one of the NTCP binding assays known in the art, or as exemplified herein (see Example section). Compounds of the present invention preferably have an affinity for NTCP, preferably the inward-facing or open pore confirmational state, sufficient to provide adequate binding for the intended purpose. Suitably, such compounds for instance have an affinity (K_{d}) of from 10⁻⁵ to 10⁻¹⁵ M. For use as a therapeutic, the compound suitably has an affinity (K_{d}) of from 10⁻⁷ to 10⁻¹⁵ M, preferably from 10⁻⁸ to 10⁻¹² M and more preferably from 10⁻¹⁰ to 10⁻¹² M. As will be evident to the skilled person, the cryo-EM structure presented herein has enabled, for the first time, new conformational states and dynamics of NTCP. The skilled artisan is provided herein with the necessary tools and technical information to test the affinity, and identify as such the test compound as one that specifically binds to the NTCP target when it has a K_{D} of 10⁻⁵ M or less for NTCP binding in a binding assay. The target is in particular chosen from NTCP (SEQ ID NO: 1-4). The definition is met if the criteria is obtained for at least the NTCP target.

Another embodiment relates to the use of the binding agent disclosed herein for drug screening, or for structure-based drug design or modelling.

Using a variety of known modelling techniques, the cryo-EM structures of the present application can be used to produce models for evaluating the interaction of compounds with NTCP, in particular with the complexes of NTCP with Nbs in the inward-facing or open pore state. As used herein, the term “modelling” includes the quantitative and qualitative analysis of molecular structure and/or function based on atomic structural information and interaction models. The term “modelling” includes conventional numeric-based molecular dynamic and energy minimisation models, interactive computer graphic models, modified molecular mechanics models, distance geometry and other structure-based constraint models. Molecular modelling techniques can be applied to the atomic coordinates of the NTCP_{EM}, Nb complexes or parts thereof to derive a range of 3D models and to investigate the structure of binding sites, such as the binding sites with chemical entities. These techniques may also be used to screen for or design small and large chemical entities which are capable of binding NTCP conformational states disclosed herein and modulate the activity of NTCP. Such a screen may employ a solid 3D screening system or a computational screening system. Such modelling methods are to design or select chemical entities that possess stereochemical complementary to identified binding sites or pockets. By “stereochemical complementarity” it is meant that the compound makes a sufficient number of energetically favourable contacts with NTCP as to have a net reduction of free energy on binding. By “stereochemical similarity” it is meant that the compound makes about the same number of energetically favourable contacts with NTCP set out by the coordinates shown in PDB files: 7PQG and 7PQQ. Stereochemical complementarity is characteristic of a molecule that matches intra-site surface residues lining the groove of the receptor site as enumerated by the coordinates set out in PDB files: 7PQG and 7PQQ. By “match” we mean that the identified portions interact with the surface residues, for example, via hydrogen bonding or by non-covalent Van der Waals and Coulomb interactions (with surface or residue) which promote dissolvation of the molecule within the site, in such a way that retention of the molecule at the binding site is favoured energetically. It is preferred that the stereochemical complementarity is such that the compound has a K_{d} for the binding site of less than 10⁻⁴M, more preferably less than 10⁻⁵M and more preferably 10⁻⁶M. In a most particular embodiment, the K_{d} value is less than 10⁻⁸M and more particularly less than 10⁻⁹M.

A number of methods may be used to identify chemical entities possessing stereochemical complementarity to the structure or substructures of NTCP in the disclosed conformations. For instance, the process may begin by visual inspection of a selected binding site in NTCP on the computer screen based on the coordinates in PDB files: 7PQG and 7PQQ, generated from the machine-readable storage medium. Alternatively, selected fragments or chemical entities may then be positioned in a variety of orientations, or docked, within the selected binding site. Modelling software is well known and available in the art. This modelling step may be followed by energy minimization with standard available molecular mechanics force fields. Once suitable chemical entities or fragments have been selected, they can be assembled into a single compound. In one embodiment, assembly may proceed by visual inspection of the relationship of the fragments to each other on the three-dimensional image displayed on a computer screen in relation to the atomic coordinates of selected binding site or binding pocket in the NTCP binding site. This is followed by manual model building, typically using available software. Alternatively, fragments may be joined to additional atoms using standard chemical geometry. The above-described evaluation process for chemical entities may be performed in a similar fashion for chemical compounds.

Databases of chemical structures are available from a number of sources including Cambridge Crystallographic Data Centre (Cambridge, U.K.), Molecular Design, Ltd., (San Leandro, Calif.), Tripos Associates, Inc. (St. Louis, Mo.), Chemical Abstracts Service (Columbus, Ohio), the Available Chemical Directory (Symyx Technologies, Inc.), the Derwent World Drug Index (WDI), BioByteMasterFile, the National Cancer Institute database (NCI), Medchem Database (BioByte Corp.), ZINC docking database (University of California, Sterling and Irwin, J. Chem. Inf. Model, 2015), and the Maybridge catalogue. Once an entity or compound has been designed or selected by the above methods, the efficiency with which that entity or compound may bind to NTCP conformational states can be tested and optimised by computational evaluation. For example, a compound that has been designed or selected to function as a NTCP binding compound must also preferably traverse a volume not overlapping that occupied by the binding site when it is bound to the native NTCP. An effective NTCP binding compound must preferably demonstrate a relatively small difference in energy between its bound and free states (i.e. a small deformation energy of binding). Thus, the most efficient NTCP binding compound should preferably be designed with a deformation energy of binding of not greater than about 10 kcal/mole, particularly, not greater than 7 kcal/mole. NTCP binding compounds may interact with, for instance but not limited to, the NTCP in more than one conformation that are similar in overall binding energy. In those cases, the deformation energy of binding is taken to be the difference between the energy of the free compound and the average energy of the conformations observed when the compound binds to the protein. Further, a compound designed or selected as binding to NTCP may be further computationally optimised so that in its bound state it would preferably lack repulsive electrostatic interaction with the target protein.

Once a NTCP-binding compound has been optimally selected or designed, as described above, substitutions may then be made in some of its atoms or side groups to improve or modify its binding properties. Generally, initial substitutions are conservative, i.e. the replacement group will have approximately the same size, shape, hydrophobicity and charge as the original group. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. It should, of course, be understood that components known in the art to alter conformation should be avoided. Such substituted chemical compounds may then be analysed for efficiency of fit to the NTCP in its preferred conformation by the same computer methods described above.

Specific computer software is available in the art to evaluate compound deformation energy and electrostatic interaction. The screening/design methods may be implemented in hardware or software, or a combination of both. However, preferably, the methods are implemented in computer programs executing on programmable computers each comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design. Each program is preferably implemented in a high level procedural or object-oriented programming language to communicate with a computer system. However, the programs can be implemented in assembly or machine language, if desired. In any case, the language may be compiled or interpreted language. Each such computer program is preferably stored on a storage medium or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

It is to be understood that although particular embodiments, specific configurations as well as materials and/or molecules, have been discussed herein for methods, samples and products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Introduction

Liver diseases constitute an important human health burden, as they are the cause of significant mortality world-wide and, their incidence has steadily grown over the last decades in western societies. This is mainly due to lack of appropriate pharmaco-therapy.

The present invention relates to nanobodies (Nbs) against human sodium-taurocholate co-transporting polypeptide (NTCP_{WT}; SEQ ID NO:1), also known as Solute Carrier 10A1 (SLC10A1)⁷. NTCP_{WT} is specifically expressed in liver tissue, at the hepatocyte basolateral membrane (“blood side°) enabling uptake of bile salts (BS), and possibly also in stellate cells⁷². Moreover, NTCP_{WT} is the main cellular entry receptor of human hepatitis B and D viruses (HBV and HDV)^{2,3}. Due to its role as viral receptor and its specific expression in liver, NTCP_{WT} has emerged as an important target to treat liver diseases, and deliver therapeutics into the liver⁷²⁻⁷⁴. Thus, peptides that mimic HBV receptor recognition sequence, namely PreS1, have been shown to deliver molecular cargos into cells expressing NTCP-consistent with fast turnover at the plasma membrane⁷⁵, as well as to concentrate those cargos in liver in rodent models⁷⁶⁻⁵⁶.

In this work, we set out to study the structural basis of human NTCP function. We used cryo-electron microscopy (cryo-EM) in combination with conformation-specific nanobodies (Nbs) to reveal important conformational transitions of NTCP transport cycle.

### Example 1. Identification of NTCP Specific Nanobody Families

Human NTCP_{WT} (SEQ ID NO:1), and its N-glycosylation mutant (NTCP_{WT_KOG}; SEQ ID NO:2) are unstable in non-denaturing detergent solutions required for its purification, and further animal immunization. To overcome this problem, we exchanged amino acids in the sequence of NTCP_{WT_KOG} for consensus residues of representative vertebrate SLC10A1 orthologs to confer stability, and enable purification of folded protein samples. We engineered two so-called NTCP consensus designs or consensus constructs. The first consensus design, namely NTCP_{CO} (SEQ ID NO:3) contains 14 consensus-residue exchanges, and was used for animal immunization, as well as nanobody selection, and characterization. The second consensus design, namely NTCP_{EM} (SEQ ID NO:4) contains 7 consensus-residue exchanges, and was used for cryo-electron microscopy structure determination, as described in Example 2.

Llamas (Lama glama) were immunized with purified NTCP_{CO} (SEQ ID NO:3), and nanobody generation followed published protocols⁵⁵, and as described in the material and methods herein. Different Nb families, as defined by the difference in the CDR3, were selected by biopanning. Periplasmic extracts of different clones were made and subjected to ELISA screens, yielding 50 nanobody molecules (herein Nb_{NTCP}_XY, where XY denotes a unique two-digit number) that grouped into 25 families (herein FamN, where N denotes a number from 1 to 25) based on conservation of the amino acid sequence of the complementary-determining region 3 (CDR3). Nb87 and Nb91, containing the 6×His-EPEA C-terminal tag, were expressed in E. coli for subsequent purification from the bacterial periplasm (see methods).

Further characterization was done by probing the effect of nanobodies on bile acid transport, and/or on binding of HBV and HDV PreS1 peptide. To do this, we optimized fluorescence-based assays in HEK293 cells transiently expressing NTCP constructs (see methods) to ensure extracellular recognition of the transporters.

Furthermore, the cryo-electron microscopy (cryo-EM) structures of NTCP_{EM} (SEQ ID NO:4) in complexes with Nb_{NTCP}_87 (SEQ ID NO:5 with a C-terminal 6×His-EPEA tag as in SEQ ID NO:38), and Nb_{NTCP}_91 (SEQ ID NO: 7) (in this case with the megabody scaffold³⁰) were resolved, as described below, demonstrating that these Nbs stabilized two different conformations of NTCP transport cycle.

### Example 2. Cryo-EM Structure Determination

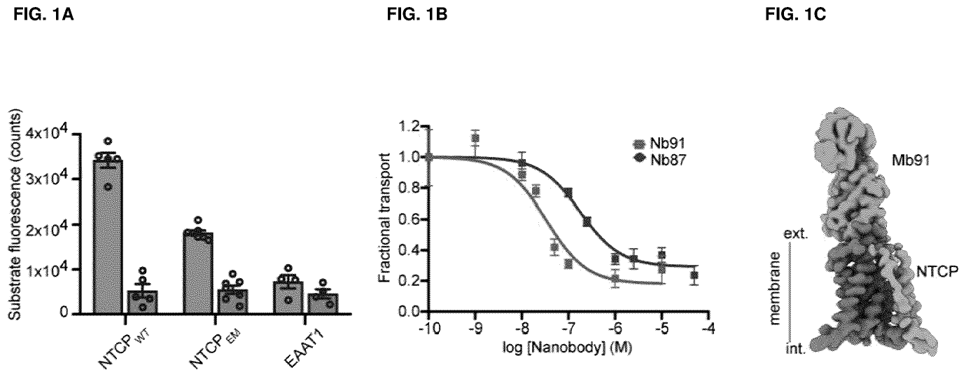

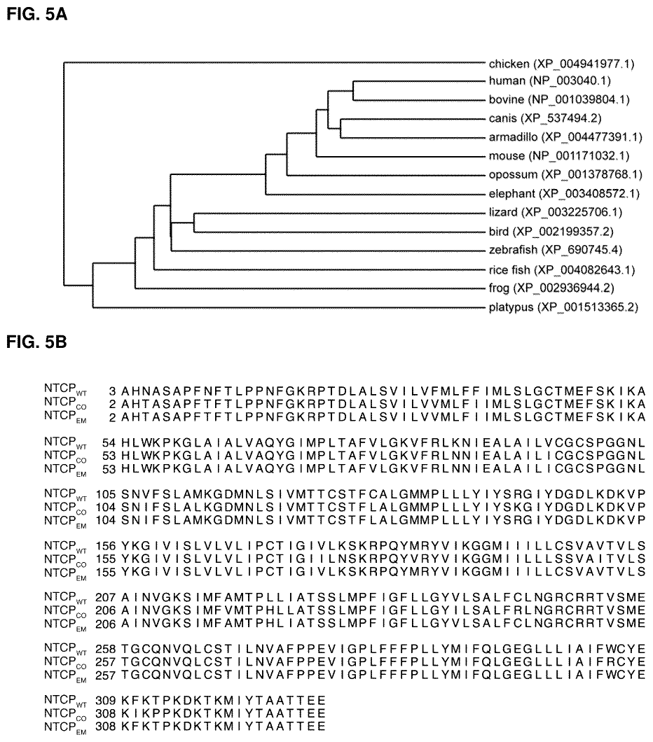

Human NTCP is a relatively small (˜38 kDa) dynamic membrane protein that lacks soluble-folded domains, and is thus biochemically unstable in non-denaturing detergent solutions, posing a significant challenge for single particle cryo-electron microscopy (cryo-EM) structure determination. To overcome these problems, we first exchanged amino acids in the sequence of wild type NTCP (NTCP_{WT}; SEQ ID NO:1) for consensus residues of representative vertebrate orthologs to confer stability (see methods)²⁹. The initial consensus design, namely NTCP_{CO} (SEQ ID NO:3), was significantly more stable than NTCP_{WT} in detergent solutions. In order to minimize the number of consensus exchanges and maximize stability, we determined the contribution of single exchanges, and retain only those that increase stability, yielding a final construct that shares ˜98% amino acid identity with NTCP_{WT} (FIG. 5), and enables purification of monodisperse material in milligram-amounts. Herein, we refer to this construct as NTCP_{EM} (SEQ ID NO:4). Indeed, NTCP_{EM} showed robust Na⁺-dependent uptake of fluorescent substrate analog tauro-nor-THCA-24-DBD (4.5±1.3-fold increase sodium- over choline-based condition), comparable to that of NTCP_{WT} (10.2±3.9), while control cells expressing unrelated Na⁺-dependent neurotransmitter transporter EAAT1 lacked BS uptake (1.6±0.1) (FIG. 1a). These functional results show that the transport mechanism of NTCP_{EM} is conserved.

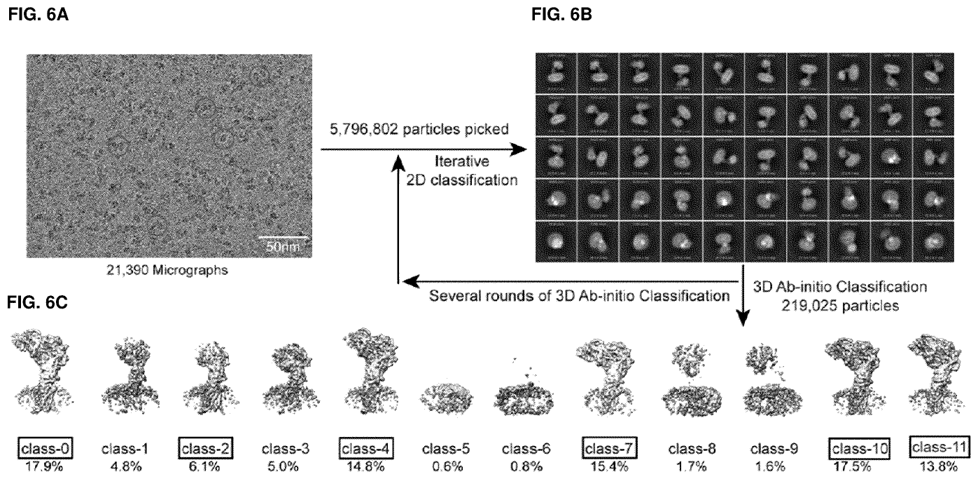

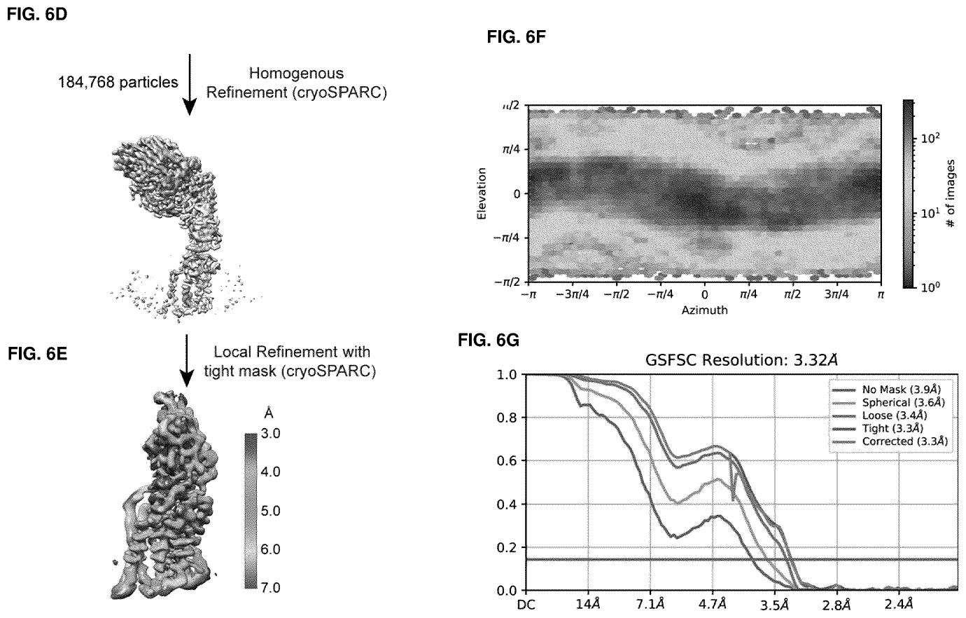

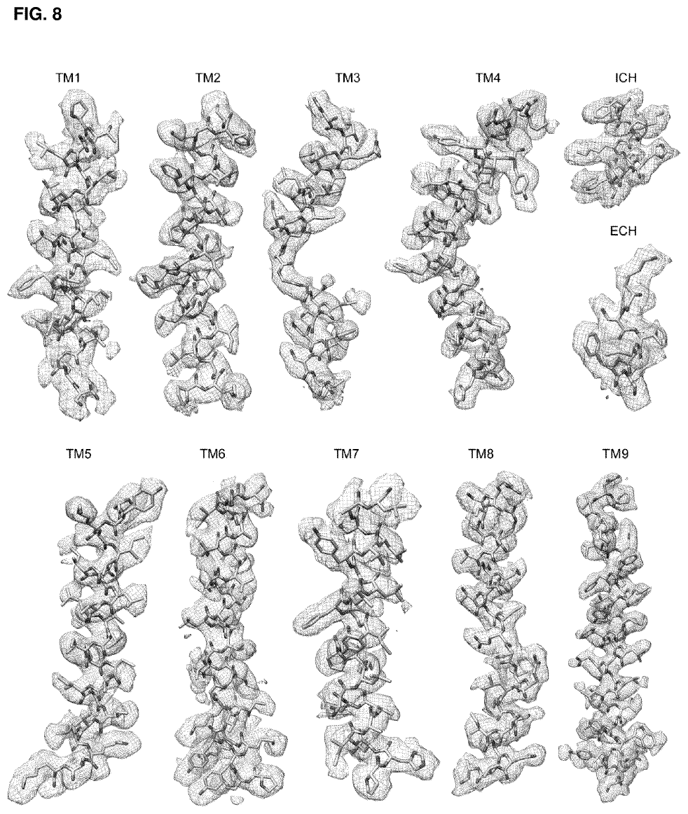

In order to provide molecular features on NTCP_{EM} surface for cryo-EM analysis, we selected Nbs that potently bind NTCP_{EM}. Nb87 and Nb91 were shown to inhibit Na⁺-induced fluorescent-substrate uptake by cells expressing NTCP_{EM} with half-maximal inhibitory concentrations (IC₅₀) of ˜180 and ˜34 nM (FIG. 1b), respectively, showing that they recognize NTCP_{EM} from the extracellular side, and suggesting that they stabilize conformational intermediates of the transport cycle. During cryo-EM sample optimization, we screened NTCP_{EM} complexes with the above-mentioned Nbs and recently-developed megabody (Mb) scaffolds that bring about additional ˜85 kDa folded domains³⁰, respectively, in both detergent solutions, as well as reconstituted in nanodisc. This yielded final cryo-EM maps of complexes NTCP_{EM}-Nb87 in nanodisc, and NTCP_{EM}-Mb91 in detergent at overall 3.7, and 3.3 Å resolution, respectively, enabling structure determination (FIG. 1c; FIG. 6-8).

### Example 3. NTCP Architecture

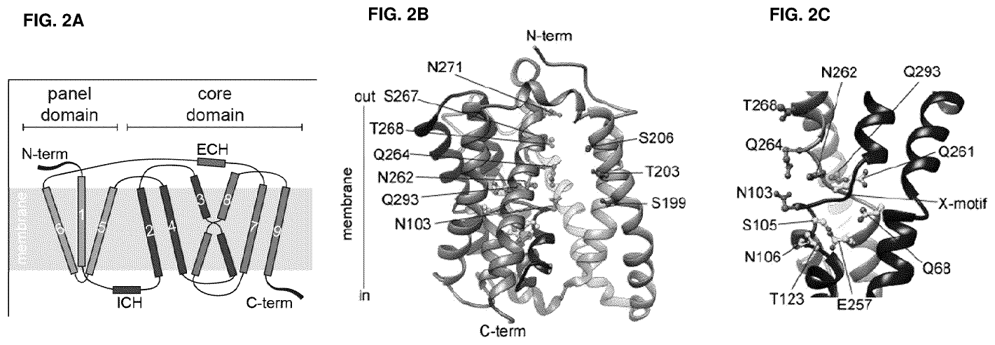

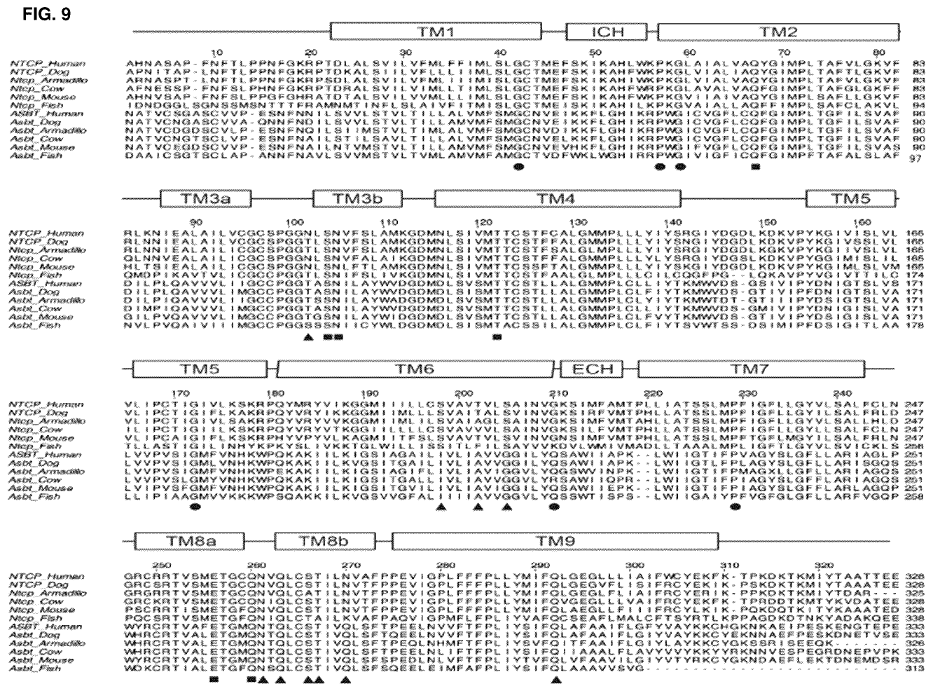

NTCP_{EM} adopts an SLC10 fold with two structurally-distinct domains, core and panel (FIG. 2a,b), and contains nine transmembrane «-helices (TM1-9) with an unstructured N-terminus on the extracellular side. The TMs are connected by short loops, as well as extra- (ECH) and intracellular (ICH) α-helices laying nearly parallel to the membrane. The panel domain is formed by TM1, TM5, and TM6, and has lost pseudo-internal symmetry compared to its equivalent in SLC10 prokaryotic homologs, due to evolutionary loss of one TM. NTCP_{EM} core domain is formed by packing of two helix-bundles, TM2-4 and TM7-9, respectively, that are related by pseudo two-fold symmetry (alpha-carbon RMSD ˜5 Å). TM3 and TM8 unwind close to the middle of the membrane, and pack against each other forming a characteristic X-shape structure that displays highly conserved polar-residue motifs among vertebrate SLC10 BSs transporters (FIG. 9, 10).

Most reported residues important for binding of sodium and substrate map to the core domain. Sodium binding sites 1 (Na1; including S105, N106, T123, and E257 sidechains), and 2 (Na2; Q68 and Q261), first observed in crystallographic studies of prokaryotic SLC10 homologs²⁷ are structurally conserved in NTCP_{EM} (FIG. 2c). Structural conservation, and reported NTCP mutagenesis^{31,32} strongly suggests that the two sodium ions thermodynamically-coupled to BS transport^{33,34} bind to these sites. Beyond Na1 and Na2, mutations at residues in the X-motif (equivalent to N262)²⁷ or in close proximity (Q293)³² impaired transport function, suggesting a role in substrate binding. Consistently, human NTCP inactivating mutation S267F³⁵, associated to hypercholanemia and vitamin D deficiency³⁶ lays just atop the X-motif, and A64T³⁷ is close to the sodium binding sites.

### Example 4. NTCP Inward-Facing State Transition to Open-Pore Conformation

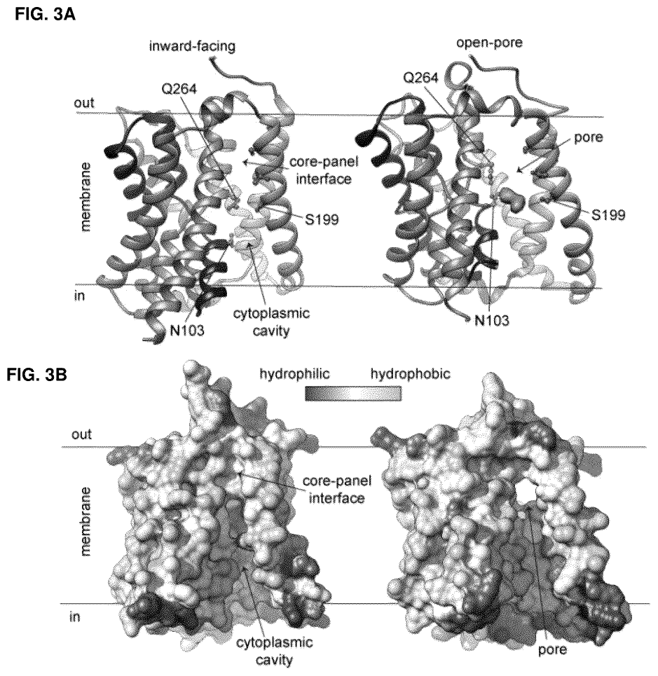

In complex with Nb87, NTCP_{EM} adopts an inward-facing state with core and panel domains tightly packing against each other on the extracellular side of the membrane (FIG. 3a,b). On the intracellular side, the domains separate uncovering an amphiphilic large cavity (molecular-surface volume >1,500 Å³) that opens to the cytoplasm, as well as laterally to the hydrophobic core of the membrane through a crevice between TM6 and TM9. On the other side of the transporter, TM1 and TM5 pack against the core domain, occluding the cavity from the membrane.

Na1 and Na2 face this cavity and localize behind the conserved X-motif. In addition, the cavity is lined by several conserved polar residues from the core domain (including, N103, N262, Q264, and Q293), some of which have been shown to be important for transport, while hydrophobic residues mostly belong to the panel domain. Amino acid conservation, mutagenesis, and its large volume suggest that the cavity is part of the substrate pathway on the cytoplasmic side. Consistently, a molecule of taurocholate bound to the equivalent region in the structure of a prokaryotic homolog has been reported²⁷.

In complex with MegaBody91 (Mb91), NTCP_{EM} Shows a remarkable conformational change compared to the inward-facing state (FIG. 3a,b). Core and panel domains rotate (˜20°) and translate (˜5 Å) towards opposite sides of the membrane nearly as rigid bodies. These movements are facilitated by conserved glycine and proline residues that act as “hinges° in connecting loops, as well as in IHC and ECH (FIG. 9). As a consequence, the two domains separate from each other on both extracellular and cytoplasmic sides, and open a wide pore through the transporter exposing Na⁺-binding sites and X-motif residues simultaneously to opposite sides of the membrane. This is a highly unexpected conformational transition, since typically active transporters alternately expose their ligand binding sites to the extra and intracellular solutions, and adopt intermediate states with substrates occluded within the protein^{38,39}. A plausible NTCP transport mechanism including an open-pore state is discussed below.

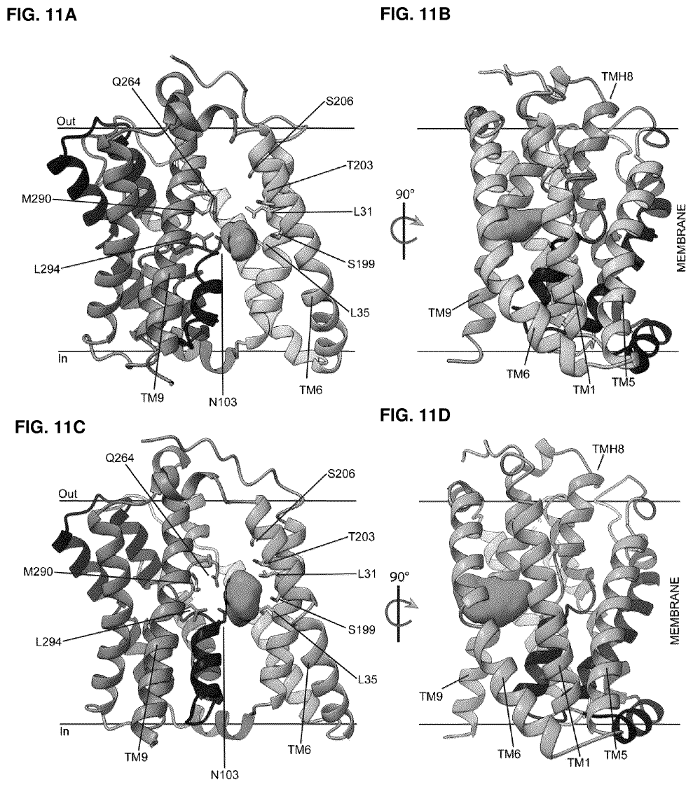

The surface lining the pore is amphiphilic, and most polar residues in this surface come from the core domain, including conserved sidechains in the X-motif. Human NTCP mutations S267F^{35,36} and S199R⁴⁰ associated to hypercholanemia also map to that surface on opposite sides of the membrane. The pore has a minimal diameter of ˜5 Å, and contains a large volume (2,400 Å³), with its long axis oriented at ˜45° angle with the membrane plane. It displays wide openings on extra and intracellular sides to bulk solutions, as well as hydrophobic membrane leaflets. Amino acid conservation, as well the architecture and amphiphilic nature of the pore strongly argue that it is the pathway for translocation of a wide, as range of amphiphilic bulky substrates transported by NTCP, including BSs^{7,41}, sulfated steroids^{42,43} well as statins^{14,15}. Consistently, in NTCP_{EM}-Mb91 cryo-EM map, we observed extra density that partly occupies the pore on the cytoplasmic side, wedged in the crevice between TM6 and TM9, in close proximity to conserved residues in the X-motifs (FIG. 3a; FIG. 11). Our cryo-EM sample included both Na⁺ and substrate taurocholate, and the density likely corresponds to a substrate molecule bound to NTCP_{EM}. However, lack of molecular features in the density precluded unambiguous determination of the bound molecule.

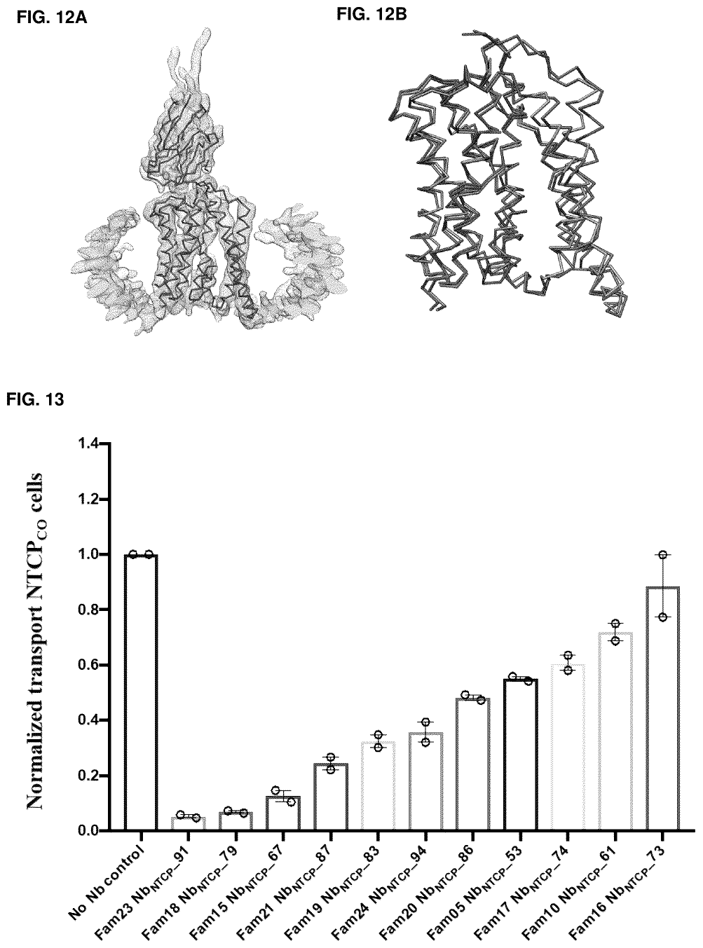

It is worth noting that NTCP_{EM}-Mb91 complex structure was determined from samples in detergent solutions, raising the possibility that detergent molecules somehow could have facilitated the open-pore state. To shed light on this question, we determined the cryo-EM structure of NTCP_{EM}-Nb91 complex reconstituted in a nanodisc. Despite the limited resolution of the cryo-EM map (˜4.3 Å), we could confidently model NTCP_{EM} in a conformation nearly identical to that observed in detergent solutions (RMSD ˜1.4 Å) (FIG. 12), demonstrating that NTCP_{EM} adopts an open-pore state in a lipid bilayer and hence, that it represents a functional state of the transport cycle. Interestingly, we also observed similar extra density that localizes to the pore in the nanodisc-reconstituted NTCP_{EM}-Nb91 complex (FIG. 11), further supporting the idea that the density corresponds to a substrate molecule, rather than detergent bound to the transporter.

### Example 5. Nb87 Impairs the NTCP Myr-PreS1 Binding

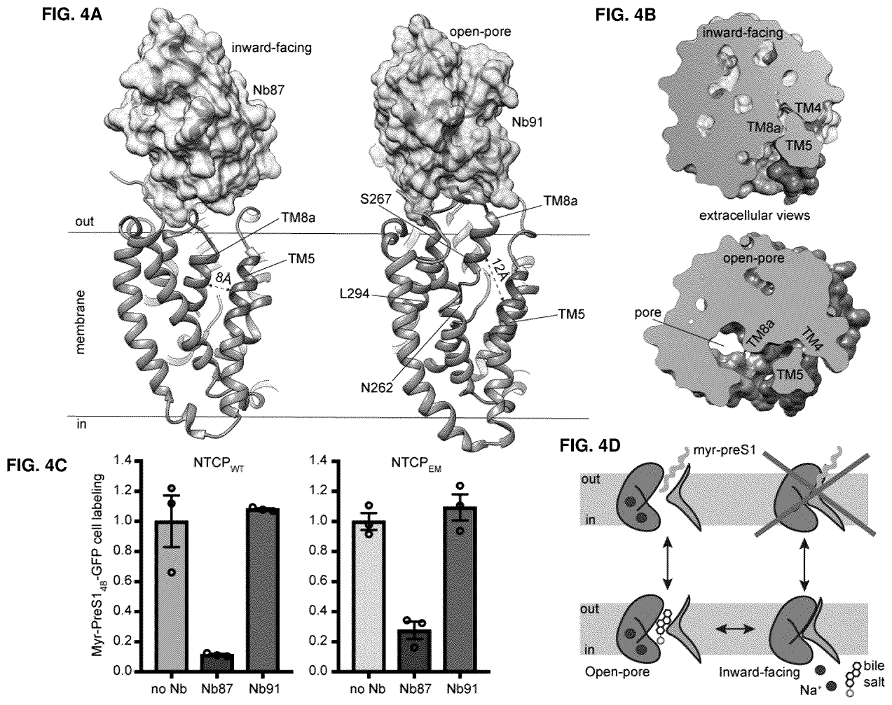

The conformational changes associated to NTCP_{EM} pore opening have important implications for HBV/HDV receptor-recognition mechanism. A reported critical region for myr-preS1 binding and viral infection (NTCP residues K157-L165)² maps to the extracellular half of TM5 in the panel domain, and localizes far (>20 Å) from both Nb87 and Nb91 binding interfaces on the surface of the core domain (FIG. 4a). Notably, there is a significant conformational change around TM5 when comparing NTCP_{EM} inward-facing and open-pore states (FIG. 4a,b). In the former, TM5 packs tightly against TM4 and TM8b in the core, generating a shallow groove at the interdomain interface lined by hydrophobic residues. In stark contrast, relative movements of core and panel domains, as well as tilting of TM5 towards the membrane in the open-pore state, unpack the extracellular part of this helix away from the core domain (by as much as ˜4 Å), creating a crevice between TM5 on one side, and TM8b and the X-motif on the other. Moreover, pore-lining residues that impair both myr-PreS1 binding and BS transport (including N262, S267, L294)³² are only accessible to the outside in the open-pore state. The changes in accessibility around critical regions for HBV/HDV recognition suggests that myr-preS1 may bind differentially to open-pore and inward-facing states.

To test this hypothesis, we optimized a fluorescence-based myr-PreS1 binding assay in cells using a purified myr-preS1₄₈ lipopeptide fused to GFP (myr-preS1₄₈-GFP). Indeed, myr-preS1₄₈-GFP labelling of cells expressing NTCP_{WT} or NTCP_{EM} was greatly decreased in the presence of Nb87, but was not affected by Nb91 (FIG. 4c). Nb87 and Nb91 overlapping epitopes on the surface of the core domain distant from HBV/HDV binding determinants strongly argues that the inhibitory effect of Nb87 on myr-preS1₄₈-GFP binding is not due to direct steric hindrance, but rather to stabilization of the inward-facing state that allosterically buries myr-preS1 binding determinants within the protein core. Overall, structural and functional results indicate that myr-preS1 preferentially binds to the open-pore state, and interacts with exposed residues lining the pore at the interface between core and panel domains.

Our structural and functional analyses of NTCP_{EM} in complexes with conformation-specific nanobodies reveal key molecular aspects of NTCP transport, and HBV/HDV receptor-recognition mechanisms.

NTCP_{EM} open-pore structure is apparently at odds with the alternating-access transport mechanisms observed in most solute carrier families^{38,39}, including SLC10 prokaryotic homologs^{27,28}, that involve occluded substrate-bound intermediates of the transport cycle, raising the important question on how to reconcile an open-pore intermediate state with thermodynamically active transport. Our structures suggest a plausible mechanism, whereby the pore is transiently opened in the presence of substrate—and thermodynamically coupled Na⁺—, and it closes upon release of ligands into the cytoplasm in the inward-facing state (FIG. 4d). Presence of extra cryo-EM density in the pore, likely representing a BS molecule bound to the transporter, supports such mechanism. Moreover, to avoid BS permeation downhill its electrochemical gradient, sodium ions should contribute to gate the pore, and preferentially enable BS binding at high extracellular sodium concentrations from outside (under physiological ionic gradients), for instance by inducing outward-facing states that resemble those observed in SLC10 prokaryotic homologs²⁸. Along this line of thinking, early ion transport theories considered active carriers as pores, whose gates are controlled by the energy source^{44,45}, challenging the classical distinction between channels and pumps⁴⁶. To the best of our knowledge, the NTCP open-pore state is the first structural demonstration of an active transporter displaying a wide-open pore transport pathway for a bulky solute. Details on how the NTCP pore is gated on the extracellular side will require further structural and biophysical work.

The NTCP_{EM} open-pore structure further shows that HBV/HDV-binding determinants line the pore within the membrane plane, accessible to the outside, and overlap with the substrate transport pathway. In sharp contrast, the inward-facing state shows tight packing of core and panel domains on the extracellular side burying virus-binding determinant residues within the protein core and consistently, Nb87 antagonizes myr-preS1 binding. These results converge to suggest that myr-preS1 interacts with residues in the pore and hence, that HBV/HDV selectively recognize NTCP conformations with open-to-outside substrate pathway, while binding to inward-facing states is impaired (FIG. 4d). Such mechanism explains the reported inhibitory effect of myr-PreS1 binding on BS transport³², as bound myr-PreS1 would stabilize open-to-outside states and preclude isomerization into inward-facing ones, as well as the antagonism between myr-preS1 and substrate binding³², as both ligands would interact with overlapping binding sites within the pore.

The inhibitory effect of Nb87 on myr-preS1 binding uncovers the therapeutic potential of molecules that stabilize NTCP inward-facing state(s), as allosteric viral cell entry inhibitors. Such molecules could constitute alternative and/or synergistic therapeutic tools to existing lipopeptides that mimic high-affinity myr-preS1 binding^{23,47}, as well as neutralizing antibodies against HBV^{48,49}.

### Example 6. Nb_{NTCP} Families and Nb Variants Obtained by Structure-Based Mutagenesis

The most relevant NTCP binding molecules that we have identified with therapeutic potential involve seven different Nanobody families which are classified as to provide for one of the two types of conformational binders described herein.

The first type comprising the bile salt transport inhibitors that enable HBV/HDV recognition by enabling PreS1 binding. These include Nbs from Fam23, Fam18, Fam19, and Fam17, with the best characterized member of this group Nb_{NTCP}_91 (SEQ ID NO:7) belonging to Fam23. These molecules likely stabilize open-pore or open-to-outside states of the NTCP transport cycle.

The second type comprising the bile salt transport inhibitors that preclude HBV/HDV recognition by inhibiting PreS1 binding These include Nbs from Fam21, Fam05, and Fam15, with the best characterized member of this group Nb_{NTCP}_87 (SEQ ID NO:5). These molecules likely stabilize inward-facing states of NTCP transport cycle that bury PreS1 binding site within the protein core.

Moreover, based on the structural analysis of Nb-NTCP complexes, mutagenesis to Nb variants with improved properties for therapeutic applications are provided herein.

The different families and variants of Nbs disclosed herein include:

### Nb_{NTCP} Family 23 (Fam23):

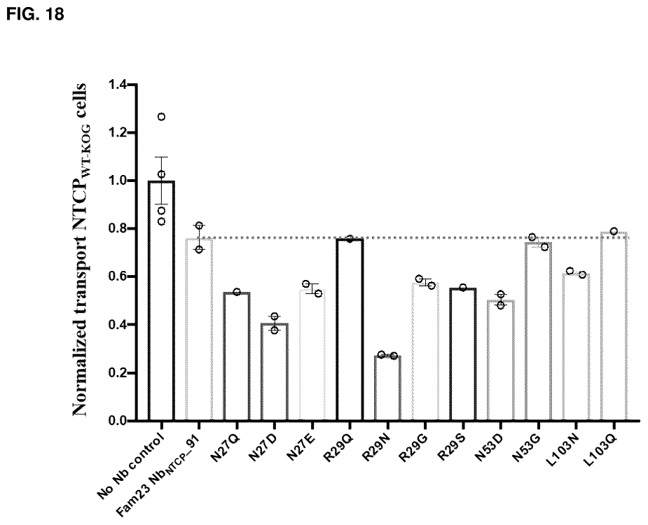

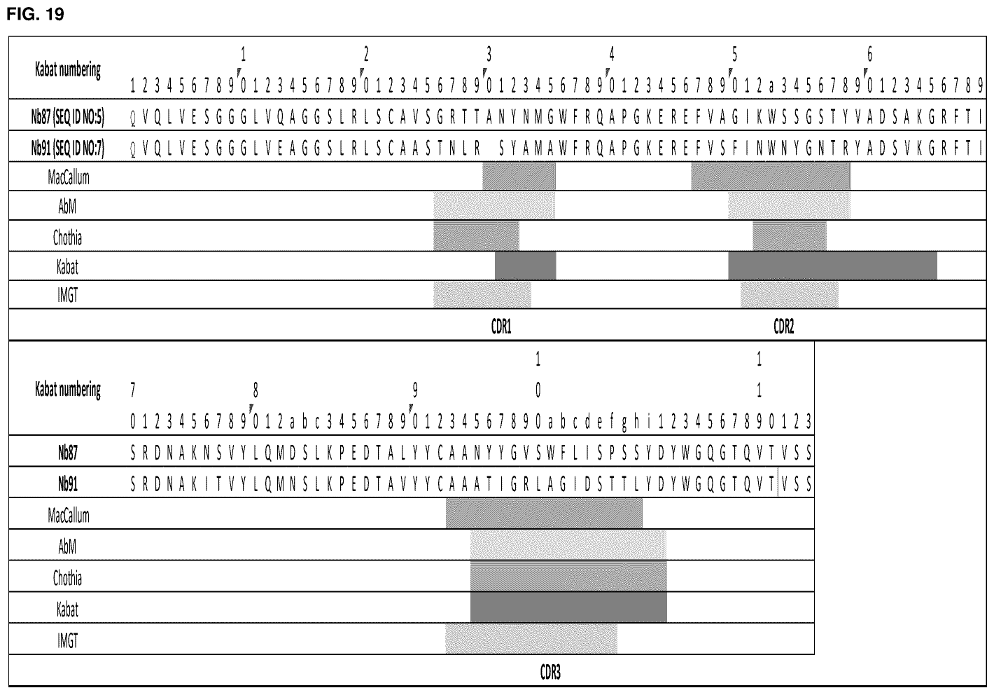

A single member was identified for this family, namely Nb_{NTCP}_91 (SEQ ID NO:7). Nb_{NTCP}_91 inhibited substrate uptake (FIGS. 13, 18), but not PreS1 binding (FIG. 15, 4c). As described in examples 2 and 4, the cryo-electron microscopy structure of Nb_{NTCP}_91 in complex with NTCP_{EM} (SEQ ID NO:4) (FIG. 3) showed that this nanobody stabilizes an open-pore of the transport cycle that is competent for PreS1 binding and HBV/HDV recognition. Based on that structure, we designed Nb_{NTCP}_91 single amino acid mutants (including N27D, N27Q, N27E, R29N, R29G, R29S, N53D, L103N, as present in SEQ ID NOs: 30-37) that outcompete binding of Nb_{NTCP}_91 (FIG. 18) indicating higher affinity of the single mutants.

### Nb_{NTCP} Family 18 (Fam18):

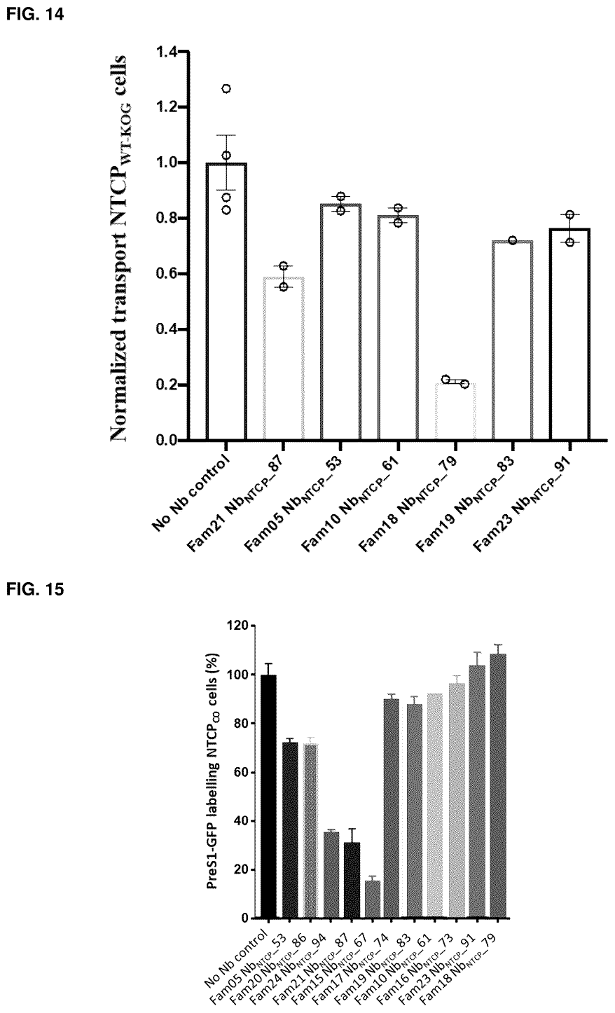

Nb_{NTCP}_79 (SEQ ID NO:8) inhibited substrate uptake (FIGS. 13, 14), but not PreS1 binding (FIG. 15). Nanobody Fam18 also includes Nb_{NTCP}_80 (SEQ ID NO:9), and Nb_{NTCP}_81 (SEQ ID NO:10) that are expected to behave in a similar way.

### Nb_{NTCP} Family 19 (Fam19):

Nb_{NTCP}_83 (SEQ ID NO:11) inhibited substrate uptake (FIG. 13), but not PreS1 binding (FIG. 15). Nanobody Fam19 also includes Nb_{NTCP}_82 (SEQ ID NO: 12), and Nb_{NTCP}_84 (SEQ ID NO:13) that are expected to behave in a similar way.

### Nb_{NTCP} Family 17 (Fam17):

Nb_{NTCP}_74 (SEQ ID NO:15) inhibited substrate uptake (FIG. 13), but not PreS1 binding (FIG. 15). Nanobody Fam17 also includes Nb_{NTCP}_75 (SEQ ID NO: 16), Nb_{NTCP}_76 (SEQ ID NO: 17), Nb_{NTCP}_77 (SEQ ID NO: 18) that are expected to behave in a similar way.

### Nb_{NTCP} Family 21 (Fam21):

Nb_{NTCP}_87 (SEQ ID NO:5) inhibited both substrate uptake (FIG. 13, 14), and PreS1-peptide binding (FIG. 4c, 15,16). As described in examples 2 and 4, the cryo-electron microscopy structure of Nb_{NTCP}_87 in complex with NTCP_{EM} (SEQ ID NO:4) (FIG. 3) showed that this nanobody stabilizes an inward-facing state of the transport cycle that is not competent for PreS1 binding and HBV/HDV recognition. Based on that structure, we designed Nb_{NTCP}_87 single amino acid mutants (including S55Q, S55E, S104G, A30Q, and S111R, as present in SEQ ID NOs: 25-29) that outcompete binding of Nb_{NTCP}_87 (SEQ ID NO:5) (FIG. 17) indicating higher affinity of the single mutants. Finally, Fam21 also includes Nb_{NTCP}_88 (SEQ ID NO:6) that is expected to behave in a similar way to Nb_{NTCP}_87 (SEQ ID NO:5).

### Nb_{NTCP} Family 05 (Fam05):

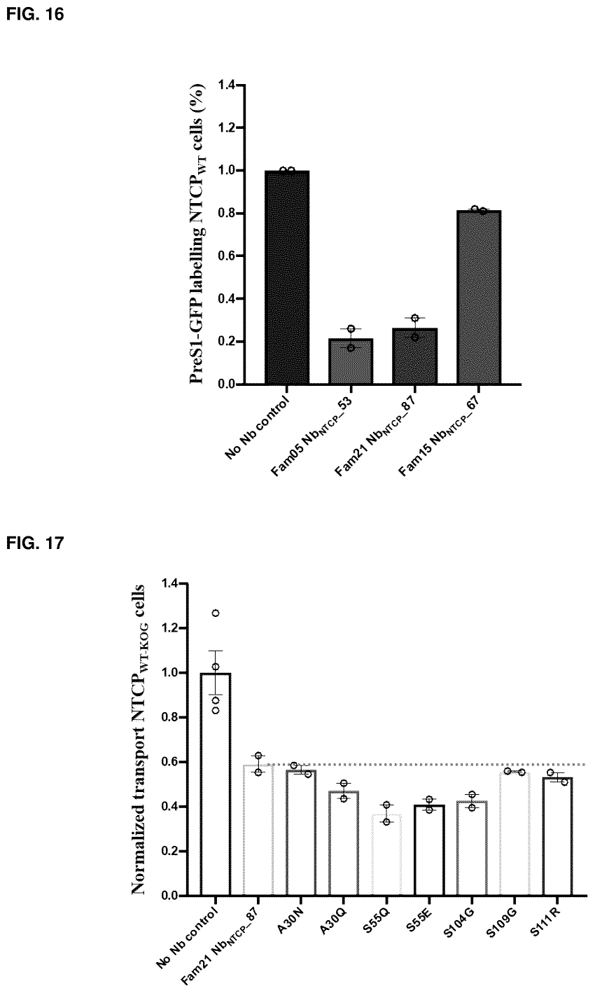

This nanobody family has a single member, namely Nb_{NTCP}_53 (SEQ ID NO:14). Nb_{NTCP}_53 inhibited substrate uptake (FIGS. 13, 14), as well as PreS1 binding (FIGS. 15, 16).

### Nb_{NTCP} Family 15 (Fam15):

Nb_{NTCP}_67 (SEQ ID NO:20) inhibited both substrate uptake (FIG. 13), and PreS1-peptide binding (FIG. 15, 16). Nanobody Fam15 also includes Nb_{NTCP}_66 (SEQ ID NO: 19), Nb_{NTCP}_68 (SEQ ID NO:21), Nb_{NTCP}_69 (SEQ ID NO:22), Nb_{NTCP}_70 (SEQ ID NO:23), and Nb_{NTCP}_71 (SEQ ID NO:24).

Although some of these molecules have been tested only with NTCP_{CO} (SEQ ID NO:3) consensus mutant, based on the structural data, it is expected that they will also bind to NTCP_{WT} (SEQ ID NO:1). In addition to improving potency of the parent nanobodies through structure-based mutagenesis, multivalent or multi-specific formats are also tested to analyze their potency in conformational binding to NTCP_{WT}.

### Example 7. Bivalent Nb Production and Humanization of Nb87 and Nb91

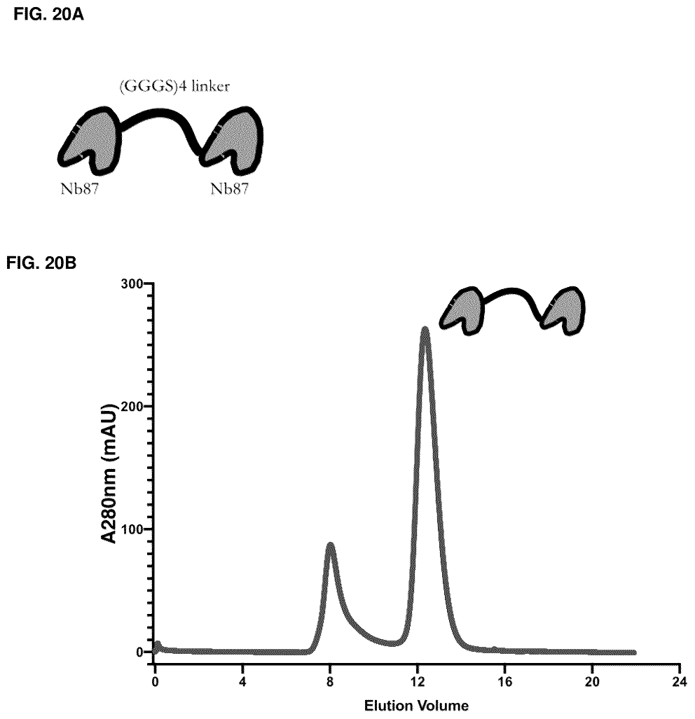

In order to improve avidity of Nb87 for human wildtype NTCP expressed on the cell surface, we engineered a bivalent molecule, namely Nb87-(GGGS)x4-Nb87 (SEQ ID NO: 67), concatenating two Nb87 molecules through a flexible linker (FIG. 20A), with in addition an N-terminal signal peptide (SEQ ID NO: 68) and a C-terminal His-EPEA affinity tag (SEQ ID NO: 38). Purified Nb87-(GGGS)x4-Nb87 bivalent Nb showed a symmetric size-exclusion chromatographic profile under preparative conditions, demonstrating good stability, homogeneity, and purity (FIG. 20B).

In order to develop the most promising Nb candidates further into biologicals for use in treatment of human subjects, the Nb sequences are known to require humanization substitutions, as described previously herein, and as known by the skilled person. As a non-limiting example of a humanization, a number of humanized variants is provided herein for our best characterized Nbs: Nb87 (SEQ ID No:5) and Nb91 (SEQ ID No:7), also applicable to the mutants disclosed herein (as provided in SEQ ID NO:25-29 and SEQ ID NO:30-37, resp.). The Nb87 humanized variants disclosed herein have one or more substitutions in the framework regions, and specifically the following residues are in scope: (according to Kabat numbering): residue Q1, A14, V59, A63, S77, D82a, K83, and/or Q108, which may be substituted to the ‘human-like’ reside as present in the human IGHV3-JH consensus (SEQ ID NO: 69), resulting for instance in a VHH sequence with at least one or more of the following substitutions: Q1E, A14P, V59Y, A63V, S77T, D82aN, K83R, and/or Q108L, exemplified herein by providing the humanized variants 1-4 of Nb87 (SEQ ID NOs: 70-73). The Nb91 humanized variants disclosed herein have one or more substitutions in the framework regions, and specifically the following residues are in scope: (according to Kabat numbering): residue Q1, E13, A14, I76, K83, and/or Q108, which may be substituted to the ‘human-like’ reside as present in the human IGHV3-JH consensus (SEQ ID NO: 69), resulting for instance in a VHH sequence with at least one or more of the following substitutions: Q1E, E13Q, A14P, I76N, K83R, and/or Q108L, exemplified herein by providing the humanized variants 1-2 of Nb91 (SEQ ID NOs: 74-75). The variants comprise CDR regions which are identical to the originally identified llama VHHs, or alternatively, identical to the mutant VHHs with retained function, as disclosed herein, but have been modified in line with human germline sequences, in line with and based on expertise of the skilled person showing which substitutions are critical for good pharmacological profiling and improved biophysical properties of the Nbs. Finally, said humanized variants of the Nb sequences as proposed and as can be designed based on the data presented herein, may be used in mono- or multivalent format, depending on the intended use or application, and optionally linked or fused with a further binding moiety or functional moiety, such as a half-life extension molecule. Non-limiting examples of the use of such humanized Nb sequences include VHH-Fc or antibody fusions, as known in the art.

### Example 8. Nb87 Binds Cell-Surface Expressed NTCP

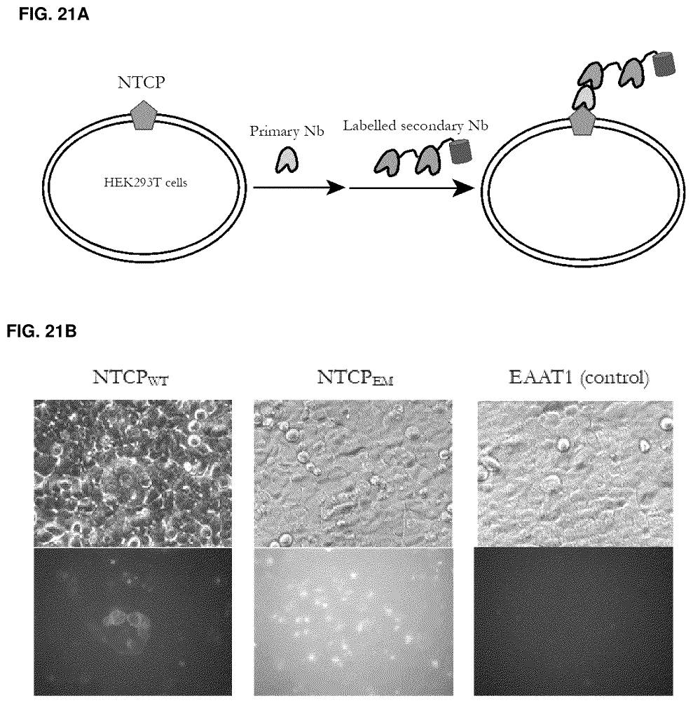

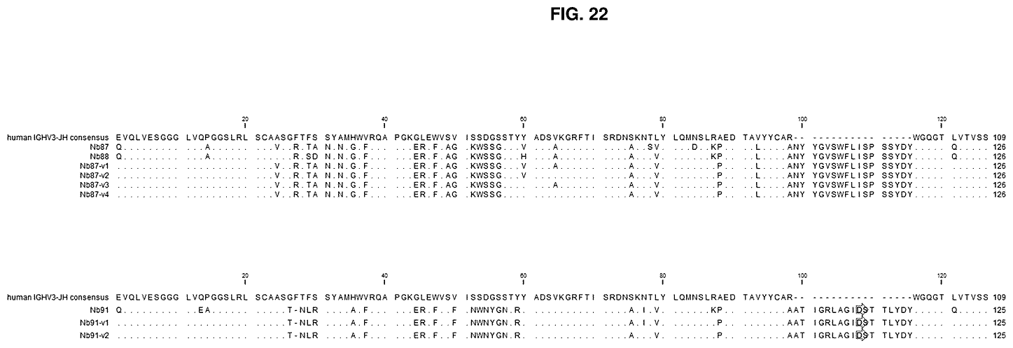

HEK293 cells transfected with human wildtype NTCP or the consensus design construct used for cryo-EM structure determination (NTCP_{EM}) were readily labelled using purified Nb87 as the primary Nb, and purified AntiC2-Nb-mCherryXL (SEQ ID NO: 76) as the secondary Nb (FIG. 21). Indeed, cells transfected with an unrelated neurotransmitter transporter (EAAT1) that is not recognized by Nb87, as a negative control, lacked fluorescence labeling. These results demonstrate that the Nb87 can be used to selectively label cells expressing human NTCP on the plasma membrane, and that Nb87 is capable of binding native cell-expressed NTCP protein.

### Materials and Methods

### Thermostable NTCP Constructs

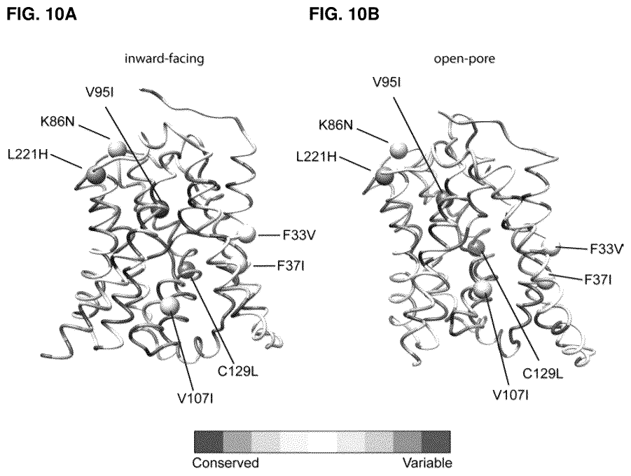

Consensus amino acids were calculated using JALVIEW⁵⁰ and reported criteria²⁹ from sequences of representative NTCP vertebrate orthologs (FIG. 5), aligned using Muscle⁵¹. Consensus amino acid exchanges were simultaneously introduced into NTCP_{WT} sequence (SEQ ID NO:1) background with N-glycosylation mutations N5T and N11T (NTCP_{WT_KOG}; SEQ ID NO:2), significantly improving protein stability. Deletions of N-terminal residue E2, and the unstructured C-terminus (residues T329-A349) in the consensus non-glycosylated construct further improved homogeneity of the sample, yielding the so-called NTCP_{CO} (SEQ ID NO:3). In general, the consensus approach generates protein samples with overall improved stability, but it is expected that by simultaneously introducing all consensus mutations, some destabilizing exchanges are included. In order to minimize the latter, we probed thermal stability of single-point NTCP_{CO} mutants, in which we reverted consensus amino acids to WT, using fluorescence-detection size-exclusion chromatography⁵². Removal of destabilizing consensus exchanges in NTCP_{CO}, yielded a consensus design, namely NTCP_{EM} (SEQ ID NO:4), that is nearly identical to NTCP_{WT} (˜98%) (FIGS. 5, 10), while preserving Na⁺-dependent BSs transport, as well as myr-PreS1 recognition mechanisms.

### Protein Expression and Purification

cDNA encoding NTCP constructs were synthesized (GenScript) and subcloned into a pcDNA3.1(+) vector encompassing a C-terminal PreScission site, followed by GFP, and two Strep-tags in tandem for affinity purification. Protein expression was done in HEK-293F (Thermo Fisher; cells were not authenticated, or tested for mycoplasma contamination) by transient transfection, as described before⁵³ with small variations. Briefly, cells grown in FreeStyle™ 293 medium (ThermoScientific™) were transfected with linear 25K polyethyleneimine (PEI, Polysciences, Inc.) at a cell density of 2.5 10⁶ cells/ml using 3 μg/ml of DNA. Valproic acid (VPA) was added to the culture at a final concentration of 2.2 mM, 6-12 h after transfection, and cells were grown for additional 48 h before harvesting.

Cell pellets were resuspended and lysed in buffer containing: 50 mM HEPES pH 7.4, 200 mM NaCl, 5% v/v glycerol, and 1 mM EDTA, 1 mM TCEP, 0.5 mM SodiumTaurocholate, and supplemented with protease inhibitors (1 mM PMSF, and protease-inhibitor cocktail from Sigma), 1% Dodecyl-β-D-Maltopyranoside (DDM, Anatrace), and 0.2% cholesteryl hemi-succinate tris salt (CHS, Anatrace), and incubated for one hour. Cell debris were removed by ultracentrifugation. Detergent-solubilized transporters were purified by affinity chromatography using streptactin sepharose resin (Cytiva Life Sciences). Resin was pre-equilibrated in buffer A containing: 50 mM HEPES pH 7.4, 200 mM NaCl, 5% v/v glycerol, 0.017% DDM, 0.0034% CHS, and 0.2 mM sodium taurocholate, and incubated with transporters for one hour under rotation. Resin was extensively washed with buffer A, and protein was eluted in buffer B containing: 50 mM HEPES pH 7.4, 200 mM NaCl, 5% v/v glycerol, 0.017% DDM, 0.0034% CHS, 0.2 mM sodium taurocholate, and 2.5 mM desthiobiotin. The eluted protein was digested with PreScission protease overnight, concentrated to several mg/ml using 100 kDa MWCO concentrator (Corning® Spin-X® UF concentrators) and injected in a Superose 6 column (GE Healthcare Life Sciences) using SEC buffer containing: 20 mM HEPES pH 7.4, 100 mM NaCl, 0.017% DDM, 0.0034% CHS, and 0.2 mM sodium taurocholate. Purified transporters were immediately used, or flash frozen and stored at −80° C. All purification steps were done at 4° C.

NTCP_{EM} complexes with nanobodies and megabodies, respectively, were formed by mixing purified protein samples at 1:1.2 (transporter:nanobody, or megabody) molar ratio, and incubated for 2 h at 4° C. Excess nanobody/megabody were removed by SEC using the above-mentioned buffer. MSP1D1 nanodisc-scaffold protein was expressed and purified using published protocols⁵⁴. Reconstitution was done by mixing purified NTCP_{EM}-Nb and -Mb complexes, respectively, with MSP1D1 and liver total lipid extract (Avanti Polar Lipids) at 0.1:1:15 molar ratio, and incubated with methanol-activated biobeads for 2 hours. Biobeads were exchanged once, and the mixture was further incubated overnight. Nanodisc-reconstituted sample was purified in a Superdex 200 increase column (GE Healthcare Life Sciences) in buffer containing: 20 mM HEPES pH 7.4, 100 mM NaCl, and 0.2 mM sodium taurocholate. Samples were concentrated as described above, and immediately used for cryo-EM grid preparation.

### The (AntiC)2-Nb-mCherryXL Fusion Protein and Bivalent Nb87 Protein Production and Purification.

The (AntiC)2-Nb-mCherryXL fusion protein (SEQ ID NO:76) contains a bivalent Nb specifically recognizing the C-terminal EPEA tag (U.S. Pat. No. 9,518,084B2; EP2576609B1), fused to the fluorescent mCherry protein label for detection, and was used in the cell surface labelling assay. The bivalent Nb production of Nb87 presents a head-to-tail fusion of Nb87 coupled by a GS-linker (SEQ ID NO: 67).

Transfection of HEK293F cells was done with 2 μg/mL DNA. Valproic acid was added and cells were diluted 1:1 with fresh media 6-8 h post transfection. Incubation was done at 37° C. for 5 days post transfection. The supernatant was collected and the pellet discarded. 250 ml of supernatant was used for binding with 3 mL of Ni-NTA beads in cell pellet cups (250 mL) for >3 h to overnight. Ni-NTA beads were collected, washed with 5 column volumes of wash buffer (50 mM Tris, 200 mM NaCl), and eluted with 4 column volumes of elution buffer (wash buffer with 250 mM Imidazole). Subsequently the eluted protein fraction was desalted, concentrated, and 10% glycerol was added before flash freezing and storage at −80° C.

### Nanobody Generation, Expression, and Purification

Nanobodies against NTCP_{CO} were generated using published protocols⁵⁵. Briefly, one llama (Lama glama) was six times immunized with a total 0.9 mg of NTCP_{CO} reconstituted in proteoliposomes. Four days after the final boost, blood was taken from the llama to isolate peripheral blood lymphocytes. RNA was purified from these lymphocytes and reverse transcribed by PCR to obtain the cDNA of the ORFs coding for the nanobodies. The resulting library was cloned into the phage display vector pMESy4 bearing a C-terminal hexa-His tag and a CaptureSelect sequence tag (Glu-Pro-Glu-Ala or EPEA), the double tag as present in SEQ ID NO: 38. Different nanobody families, as defined by the difference in the CDR3, were selected by biopanning. For this, NTCP_{CO} reconstituted in proteoliposomes was solid phase coated directly on plates. NTCP_{CO} specific phage were recovered by limited trypsinization, and after two rounds of selection, periplasmic extracts were made and subjected to ELISA screens. Nb87 and Nb91 were expressed in E. coli for subsequent purification from the bacterial periplasm. After Ni-NTA (Sigma) affinity purification, Nbs were further purified by SEC in buffer: 10 mM HEPES pH 7.4, and 110 mM NaCl.

Nb91 was enlarged by fusion to the circular permutated glucosidase of E. coli K12 (YgjK, 86 kDa) to build the megabody referred to as Mb91. Mb91 was generated and purified using reported protocols³⁰.

### Fluorescent Substrate-Analog Transport Assay

Sodium-dependent substrate uptake was measured in HEK-293F cells transfected with 2 μg/ul cDNA using the above-mentioned protocol with small modifications. 48 hours after transfection, ˜1 million cells were pelleted, washed, and resuspended in 500 μl of transport buffer (110 mM NaCl, 4 mM KCl, 1 mM MgSO4, 1 mM CaCl2), 45 mM mannitol, 5 mM glucose, and 10 mM HEPES pH 7.4), or control buffer in which NaCl was substituted by choline chloride (ChCl). To probe the effect of Nbs on BS transport, cells were incubated with a final concentration of 10 μM purified Nb for 1:30 h, followed by addition of fluorescent substrate analog tauro-nor-THCA-24-DBD^{56,57} (tebu-bio) to a final concentration of 10 μM for 30 minutes at 37° C. Excess fluorescent-analog was removed by centrifugation (13,000 g for 30 s), and one wash with the above-mentioned control buffer. Then, cells were resuspended and lysed using Pierce IP lysis buffer (Thermo Fisher). Finally, lysates were centrifuged (13,000 g for 10 minutes), and transferred to black 96-well flat-bottom plates (Grenier), and fluorescence quantified in a micro-plate reader (CLARIOstar-Plus) using λ_{excitation} 454 nm and λₑₘᵢₛₛᵢₒₙ 570 nm. Three biologically independent experiments were quantified in triplicate samples. Nb titrations data were fitted in Prism 8.0.1 (GraphPad) to the following dose-respond curve:

$$Fractional transport = 1 + \frac{( Y min - 1 )}{1 + 1 {0}^{[ log ( lC 50 ) - x ]}}$$

Where Ymin corresponds to fraction of transport at saturating Nb concentrations, IC₅₀ is half-maximal inhibitory concentration, and x is log([Nb]).

### Myr-PreS1 Purification and Binding Assay

cDNA encoding N-terminal myristoylated 2-48 consensus residues of human HBV myr-PreS1 domain (myr-GTNLSVPNPLGFFPDHQLDPAFRANSNNPDWDFNPNKDHWPEANKVG as present in SEQ ID NO:39) was synthesized (GenScript) and subcloned into a pcDNA3.1(+) vector encompassing a C-terminal GFP, and poly Histidine-tag (namely, myr-PreS1₄₈-GFP). Myr-PreS1₄₈-GFP expression was done in HEK-293F (Thermo Fisher) by transient transfection, as described for expression of NTCP_{EM} purification. Cells were lysed by 3-5 passes through a homogenizer (EmulsiFlex-C5, Avestin) and membrane fraction was collected by ultracentrifugation. Membranes were resuspended in a buffer containing: 50 mM HEPES pH 7.4, 200 mM NaCl, 5% v/v glycerol, and 1 mM EDTA, protease inhibitors (1 mM PMFS, and protease-inhibitor cocktail from Sigma), 1% Dodecyl-β-D-Maltopyranoside (DDM, Anatrace), and 0.2% cholesteryl hemi-succinate tris salt (CHS, Anatrace), and incubated for one hour. Solubilized myr-PreS1₄₈-GFP was subjected to ultracentrifugation and then purified by affinity chromatography using anti-His affinity resin (Sigma). Resin was pre-equilibrated in a buffer containing: 50 mM HEPES pH 7.4, 200 mM NaCl, 5% v/v glycerol, 0.013% DDM, 0.0027% CHS, and incubated with detergent-solubilized myr-PreS1₄₈-GFP for one hour under rotation. Resin was extensively washed with buffer containing: 50 mM HEPES pH 7.4, 200 mM NaCl, 5% v/v glycerol, 0.013% DDM, 0.0027% CHS, and 50 mM imidazole). Myr-PreS1₄₈-GFP was eluted in buffer containing: 50 mM HEPES pH 7.4, 200 mM NaCl, 5% v/v glycerol, 0.013% DDM, 0.0027% CHS, and 250 mM imidazole. The eluted protein was concentrated to several mg/ml using 30 kDa MWCO concentrator (Corning® Spin-X® UF concentrators) and injected into a Superose 6 column (GE Healthcare Life Sciences) using a SEC buffer containing: 20 mM HEPES pH 7.4, 200 mM NaCl, 0.013% DDM, and 0.0027% CHS. Myristoylation of the sample was confirmed by mass spectrometry. Purified myr-PreS1₄₈-GFP was flash frozen and stored at −80° C. All purification steps were done at 4° C.

Myr-PreS1₄₈-GFP binding to NTCP constructs was assayed in HEK-293F cells, grown and transfected with 1 μg/mL DNA using the protocol described above. 48 hours after transfection, cells were washed with pre-warmed PBS, and ˜1 million cells were pelleted and resuspended in 1 ml of PBS. To probe the effect of nanobodies, cells expressing NTCP constructs were pre-incubated with 10 μM Nbs for 1:30 h. They were then labeled with 10 nM (NTCP_{WT}) or 50 nM (NTCP_{EM}) purified myr-PreS1₄₈-GFP for 30 minutes. Excess fluorescent-probe was removed by centrifugation (13,000 g for 30 s), and one wash with PBS. Cells were then re-suspended in PBS and GFP fluorescence was recorded in a micro-plate reader (CLARIOstar-Plus) using λ_{excitation}=470 nm/λₑₘᵢₛₛᵢₒₙ=508 nm, respectively.

### Electron Microscopy Sample Preparation and Data Acquisition

Purified NTCP_{EM}-Nb or -Mb complexes were applied to glow-discharged Au 300 mesh Quantifoil R1.2/1.3. Typically, 4 μl of sample at 3-4 mg/ml were applied to the grids, and the Vitrobot chamber was maintained at 100% humidity and 4° C. Grids were screened in 200 kV Talos Arctica microscope (ThermoFisher) at IECB cryo-EM imaging facility. Final data collection was performed in 300 kV Titan Krios microscope (ThermoFisher) at EMBL-Heidelberg Cryo-Electron Microscopy Service Platform, equipped with K3 direct electron detector (Gatan). Final images were recorded with SerialEM⁵⁸ at a pixel size of 0.504 Å. Dose rate was 15-20 electrons/pixel/s.

For NTCP_{EM}-Nb87 complexes, 21,792 movies were recorded with −0.5 to −1.5 μm defocus range. Images were collected with 0.7 s subframes (total 40 subframes), corresponding to a total dose of 57.8 electrons/Å². For NTCP_{EM}-Mb91 complexes, 21,390 movies were recorded with −0.6 to −1.75 μm defocus range. Images were collected with 0.7 s subframes (total 40 subframes), corresponding to a total dose of 56.5 electrons/Å².

### Cryo-EM Data Processing, Model Building, and Structure Analysis

All data sets were processed with cryoSPARC v2 and v3⁵⁹. Movies were gain corrected, and aligned using in-built patch-motion correction routine. CTF parameters were estimated using in-built patch-CTF routine in cryoSPARC. Low-quality images were discarded manually upon visual inspection.

For NTCP_{EM}-Mb91 complex, 5,796,802 particles were template-picked from 21,390 micrographs, and selected through several rounds of 2D, as well as 3D ab initio classifications. Particles from 3D ab initio classes displaying interpretable density for transmembrane helices were pooled, and used for homogenous refinement (FIG. 6). Cryo-EM density corresponding to both detergent micelle and megabody scaffold were masked out, and particles were further subjected to local refinement using a fulcrum that localized to center of NTCP_{EM} transmembrane region. Focused refinement yielded a final map at an overall ˜3.3 Å resolution, based on the “gold-standard° 0.143 FSC cut-off.

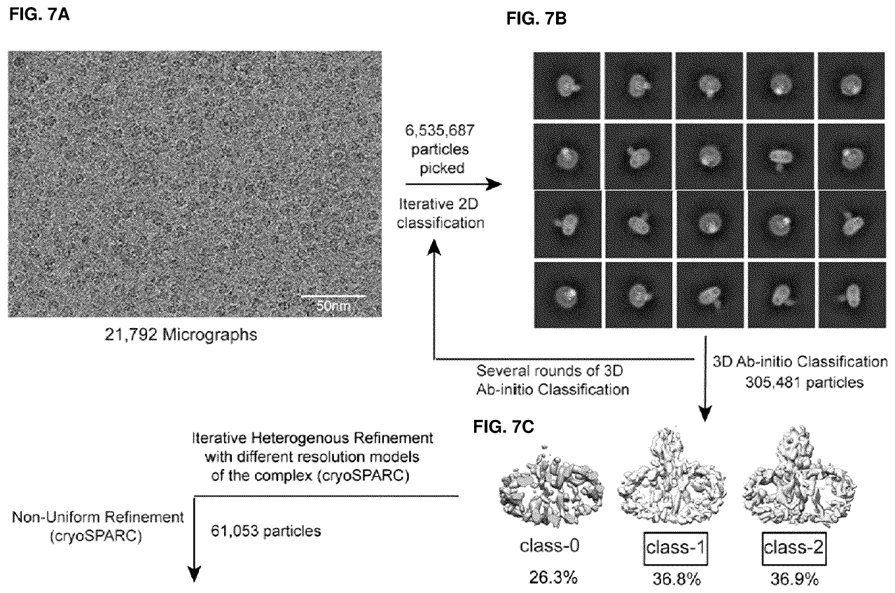

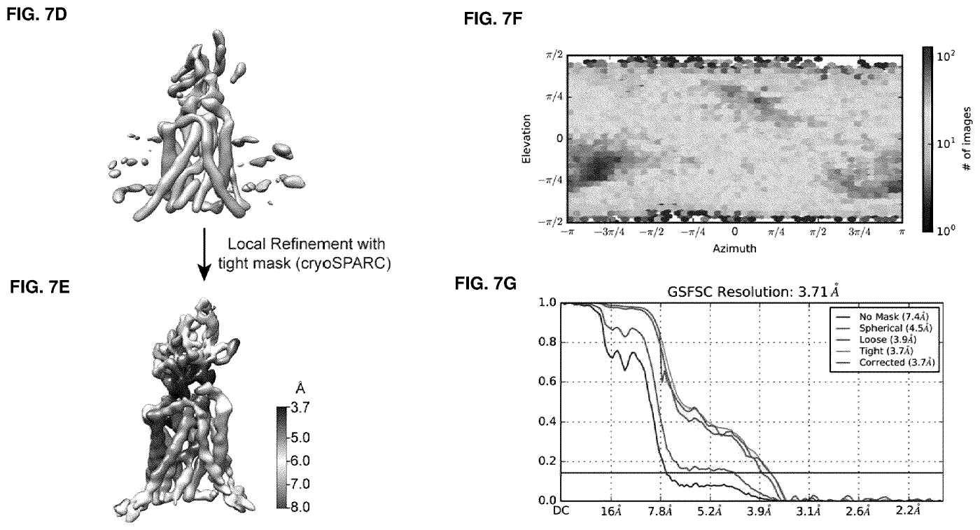

For NTCP_{EM}-Nb87 complex 6,535,687 particles were template-picked from 21,792 micrographs, and classified through several rounds of 2D, and 3D ab initio classifications (FIG. 7). ˜220,000 selected particles were further classified by heterogenous refinement yielding a final set of 61,053 particles that were processed by non-uniform refinement⁶⁰. Further focused refinement excluding nanodisc scaffold yielded a final map at an overall ˜3.7 Å resolution, based on the “gold-standard° 0.143 FSC cut-off. Maps were visualized using UCSF Chimera⁶¹ and Chimerax⁶².

Cryo-EM map of NTCP_{EM}-Mb91 complex showed clear density for most sidechains in the transmembrane helices, although TM1 and TM6 in the panel domain display less molecular features, and was used to build an atomic model of NTCP_{EM} using Coot^{63,64}. Secondary-structure predictions using Psipred⁶⁵ and bacterial homolog structure (PDB 3ZUY) were used to help initial sequence assignment. Initial Nb models were created with I-TASSER⁶⁶, and then fitted as rigid-bodies into the density, followed by manual building and modification in Coot^{63,64}. The inward-facing conformation in NTCP_{EM}-Nb87 complex was built by fitting core and panel domains from NTCP_{EM}-Mb91 structure as separate rigid-bodies into the density, followed by manual modification in coot. All atomic models were refined using PHENIX⁶⁷.

Structural analyses were carried out as follows: protein cavity calculations with CASTp 3.0⁶⁸, pore calculations MOLEonline 2.5⁶⁹, protein-protein interfaces with PISA⁷⁰, and amino acid conservation surface mapping with ConSurf⁷¹.

Data availability: Structural models of NTCP_{EM}-Nb87 and NTCP_{EM}-Mb91 complexes have been deposited in Protein Data Bank (PDB) with accession codes 7PQG and 7PQQ, respectively, and the corresponding cryo-EM maps were deposited in the Electron Microscopy Data Bank (EMDB) under accession numbers EMD-13593, and EMD-13596.

### Cell Surface Labelling Assay

Adherent HEK293T cells were seeded at 0.3×10⁶/mL using 500 μl per well in a 24-well plate and incubated for 2 days. 5 μg of cDNA coding for NTCP, or control neurotransmitter (EAAT1) was transfected as follows: (1) 5 μg DNA was mixed with 50 μl Opti-MEM and incubated for 5 min; (2)² μl lipofeactamine was mixed with 50 μl of Opti-MEM and incubated for 5 min: (3) DNA and lipofectamine solutions were mixed in 1:1 ratio and incubated for 20 min. 100 μl of the mixture was added to each well. After incubation for 6-8 h the medium was changed to DMEM medium, and incubated for 2 days post transfection. Cells were washed once with fresh medium and incubated for 1 h with 20 μM of the primary unlabeled monovalent Nb87 (containing an His-EPEA C-terminal tag). Excess of primary unlabeled Nb was removed and 10 μM of the secondary labelled Nb ((AntiC)2-mCherryXL) was added following a 1 h incubation. Cells were washed once with fresh medium prior to detection of fluorescence on an inverted microscope. Two negative controls were included: (1) cells transfected with an unrelated neurotransmitter transporter, and (2) cells labelled with the secondary Nb, but lacking the treatment with the primary nanobody Nb87.

Sequence listing

>SEQ ID NO: 1: human wild type Sodium taurocholate co-transporting polypeptide (NTCP_{WT}); Uniprot
ID Q14973; 349 amino acids
>SEQ ID NO: 2: N-glycosylation knocked-out NTCP (NTCP_{WT}_KOG); 349 amino acids
>SEQ ID NO: 3: NTCP consensus-mutant construct used for animal immunization (NTCP_{CO}); 327 amino
acids
>SEQ ID NO: 4: NTCP consensus-mutant construct used for cryo-electron microscopy structure
determination (NTCP_{EM}); 327 amino acids
Nb_{NTCP} sequences used herein, with CDRs according to Kabat annotation underlined, and amino acid
substitutions in variants in bold:
>SEQ ID NO: 5: amino acid sequence of Nb_{NTCP}_87
QVQLVESGGGLVQAGGSLRLSCAVSGRTTANYNMGWFRQAPGKEREFVAGIKWSSGSTYVADSAKGRFTISRDNA
KNSVYLQMDSLKPEDTALYYCAANYYGVSWFLISPSSYDYWGQGTQVTVSS
>SEQ ID NO: 6: amino acid sequence of > Nb_{NTCP}_88
QVQLVESGGGLVQAGGSLRLSCAASGRTSDNYNMGWFRQAPGKEREFVAGIKWSSGSTYHADSAKGRFTISRDN
AKNTVYLQMNSLKPEDTALYYCAANYYGVSWFLISPSSYDYWGQGTQVTVSS
>SEQ ID NO: 7: amino acid sequence of Nb_{NTCP}_91
QVQLVESGGGLVEAGGSLRLSCAASTNLRSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 8: amino acid sequence of Nb_{NTCP}_79
QVQLVESGGGLVQVGDSLRLSCAASGRTFSSYVMGWFRQAPGKDREFVATVSRRGINTYYADSVKGRFTISRDNAK
NTVYLQMNNLKPEDAAAYYCAADLNPKYPVTILTSDYDYWGQGTQVTVSS
>SEQ ID NO: 9: amino acid sequence of Nb_{NTCP}_80
QVQLVESGGGLVQVGDSLRLSCAASGRTFSSYVMGWFRQAPGKEREFVATISRRGINTYYADSVKGRFTISRDNAK
NTVYLQMNNLKPEDAAGYYCAADLNPKYPVTILTSDYDYWGQGTQVTVSS
>SEQ ID NO: 10: amino acid sequence of Nb_{NTCP}_81
QVQLVESGGGLVQVGDSLRLSCAASGRTFSDYVMGWFRQAPGKEREFVATVSRRGINTYYADSVKDRFTISRDNAK
NTVYLQMNNLKPEDAAAYYCAADFNPKYPLTIKTSDYDYWGQGTQVTVSS
>SEQ ID NO: 11: amino acid sequence of Nb_{NTCP}_83
QVQLVESGGGLVQPGGSLRLSCAASGSIFSLNTLGWYRQAPGKQRELVATITHGGSTKYADSVLGRFTISRDNAKNT
VYLQMYSLKPEDTAVYYCHADGGSSWFEDWPDYWGQGTQVTVSS
>SEQ ID NO: 12: amino acid sequence of Nb_{NTCP}_82
QVQLVESGGGLVQPGGSLRLSCAASGSIFSINTLGWYRQAPGKQRELVATITHGGSTKYADSVSGRFTISRDNAKNT
VYLQMNSLKPEDTAVYYCNADGGSSWFEEWPDYWGQGTQVTVSS
>SEQ ID NO: 13: amino acid sequence of Nb_{NTCP}_84
QVQLVESGGGLVQAGGSLRLSCAASGTFFSINAMGWYRQTPGQQRELVAAITSGGSTNYADSVKGRFTISRDNAK
NTVYLQMNSLKPEDTAVYYCHADGGSSWFESWDYWGQGTQVTVSS
>SEQ ID NO: 14: amino acid sequence of Nb_{NTCP}_53
QVQLVESGGGLVQAGGSLRLSCTASGRIFSWLDMAWYRQAPGKQRELVASITSVGTANYADSVKGRFTISRDNAK
NTVYLQMNSLKPEDTAVYSCNAKYSSWSGERSDWGQGTQVTVSS
>SEQ ID NO: 15: amino acid sequence of Nb_{NTCP}_74
QVQLVESGGGLVQSGGSLRLSCAASTNMVSINSMGWYRQTPGKQRELVAGATTGGSTDYADSVKGRFTISRDNA
MNTVYLQMNGLKPEDTATYYCNAIGVSRWGARYDYWGQGTQVTVSS
>SEQ ID NO: 16: amino acid sequence of Nb_{NTCP}_75
QVQLVESGGGLVQSGGSLTLSCAASVSSVSINSMAWYRQAPGKERELVAAVTSGGRIDYSDSVKGRFTISRDNAKNT
LYLQMNGLKPEDTAAYYCNLMGTSRWGARYDYWGQGTQVTVSS
>SEQ ID NO: 17: amino acid sequence of Nb_{NTCP}_76
QVQLVESGGGLVQTGGSLRLSCAASANAVRINAMAWYRQAPGKQRELVAVVTSGGNTDYGDSVKGRFTISRDNA
KKSLYLQMNSLKPEDTAVYYCNVDGTSVWGAKYDYWGQGTQVTVSS
>SEQ ID NO: 18: amino acid sequence of Nb_{NTCP}_77
QVQLVESGGGLVQTGGSLRLSCAYSASALRINTMGWYRQVPGKERESVAHITSGDTNYADSVKGRFTISRDNAKNT
VTLQMNSLKPEDTAVYYCHALGRSRWGATYDYWGQGTQVTVSS
>SEQ ID NO: 19: amino acid sequence of Nb_{NTCP}_66
QVQLVESGGGLVQAGGSLRLSCTASGLPFTSYAMGWFRQAPGKEREFVAGITSSGGSTSLADSVKGRFTISRDNAK
NTMYLQMNSLKPDDTANYYCAAQRRWGSRWYFADSDYWGQGTQVTVSS
>SEQ ID NO: 20: amino acid sequence of Nb_{NTCP}_67
QVQLVESGGGLVQAGGSLRLSCTASGLPFTSYAMGWFRQAPGKEREFVAGITPSGGSTSLADSVKGRFTISRDNAK
NTMYLQMNSLKPNDTAVYYCAAQRRWGSRWYFADSDYWGQGTQVTVSS
>SEQ ID NO: 21: amino acid sequence of Nb_{NTCP}_68
QVQLVESGGGLVQAGGSLRLSCTASGLPFTSYAMAWFRQAPGKEREFVAGINQSGGSTSLADSVKGRFTISRDNAK
NTMYLQMNSLKPDDTAVYYCAAERRWGSRWYFADSDYWGQGTQVTVSS
>SEQ ID NO: 22: amino acid sequence of Nb_{NTCP}_69
QVQLVESGGGLVQAGGSLRLSCTASGLPFTSYAMGWFRQAPGKEREFVAGITPSGGSTSLADSVKGRFTISRDNAK
NTMYLQMNSLKPDDTAVYYCAAQRRWGSRWYFADSDYWGQGTQVTVSS
>SEQ ID NO: 23 amino acid sequence of Nb_{NTCP}_70
QVQLVESGGGLVQAGGSLRLSCTASGLPFTSYAMGWFRQAPGKEREFVAGITSSGGSTSLADSVKGRFTISRDNAK
NTMYLQMNSLKPDDTADYYCAAQRRWGTRWYFADSDYWGQGTQVTVSS
>SEQ ID NO: 24: amino acid sequence of Nb_{NTCP}_71
QVQLVESGGGLVQAGGSLRLSCTASGLPFTSYAMGWFRQAPGKEREFVAGISPSGGSTSLADSVKGRFTISRDNAK
NTMYLQMNSLKPDDTAIYYCAAQRRWGTRWYFADSDYWGQGTQVTVSS
>SEQ ID NO: 25: amino acid sequence of Nb_{NTCP}_87 - S55Q variant
QVQLVESGGGLVQAGGSLRLSCAVSGRTTANYNMGWFRQAPGKEREFVAGIKWSQGSTYVADSAKGRFTISRDN
AKNSVYLQMDSLKPEDTALYYCAANYYGVSWFLISPSSYDYWGQGTQVTVSS
>SEQ ID NO: 26: amino acid sequence of Nb_{NTCP}_87 - S55E variant
QVQLVESGGGLVQAGGSLRLSCAVSGRTTANYNMGWFRQAPGKEREFVAGIKWSEGSTYVADSAKGRFTISRDNA
KNSVYLQMDSLKPEDTALYYCAANYYGVSWFLISPSSYDYWGQGTQVTVSS
>SEQ ID NO: 27: amino acid sequence of Nb_{NTCP}_87 -A30Q variant
QVQLVESGGGLVQAGGSLRLSCAVSGRTTQNYNMGWFRQAPGKEREFVAGIKWSSGSTYVADSAKGRFTISRDN
AKNSVYLQMDSLKPEDTALYYCAANYYGVSWFLISPSSYDYWGQGTQVTVSS
>SEQ ID NO: 28: amino acid sequence of Nb_{NTCP}_87- S104G variant
QVQLVESGGGLVQAGGSLRLSCAVSGRTTANYNMGWFRQAPGKEREFVAGIKWSSGSTYVADSAKGRFTISRDNA
KNSVYLQMDSLKPEDTALYYCAANYYGVGWFLISPSSYDYWGQGTQVTVSS
>SEQ ID NO: 29: amino acid sequence of Nb_{NTCP}_87 - S111R variant
QVQLVESGGGLVQAGGSLRLSCAVSGRTTANYNMGWFRQAPGKEREFVAGIKWSSGSTYVADSAKGRFTISRDNA
KNSVYLQMDSLKPEDTALYYCAANYYGVSWFLISPRSYDYWGQGTQVTVSS
>SEQ ID NO: 30: amino acid sequence of Nb_{NTCP}_91- N27D variant
QVQLVESGGGLVEAGGSLRLSCAASTDLRSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 31: amino acid sequence of Nb_{NTCP}_91- N27Q variant
QVQLVESGGGLVEAGGSLRLSCAASTQLRSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 32: amino acid sequence of Nb_{NTCP}_91- N27E variant
QVQLVESGGGLVEAGGSLRLSCAASTELRSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 33: amino acid sequence of Nb_{NTCP}_91- R29N variant
QVQLVESGGGLVEAGGSLRLSCAASTNLNSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 34: amino acid sequence of NbNTOP_91- R29G variant
QVQLVESGGGLVEAGGSLRLSCAASTNLGSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 35: amino acid sequence of Nb_{NTCP}_91- R29S variant
QVQLVESGGGLVEAGGSLRLSCAASTNLSSYAMAWFRQAPGKEREFVSFINWNYGNTRYADSVKGRFTISRDNAKI
TVYLQMNSLKPEDTAVYYCAAATIGRLAGIDSTTLYDYWGQGTQVTVSS
>SEQ ID NO: 36: amino acid sequence of Nb_{NTCP}_91-N53D variant
QVQLVESGGGLVEAGGSLRLSCAASTNLRSYAMAWFRQAPGKEREFVSFINWDYGNTRYADSVKGRFTISRDNAKI
TVYLQMNSLKPEDTAVYYCAAATIGRLAGIDSTTLYDYWGQGTQVTVSS
>SEQ ID NO: 37: amino acid sequence of Nb_{NTCP}_91- L103N variant
QVQLVESGGGLVEAGGSLRLSCAASTNLRSYAMAWFRQAPGKEREFVSFINWNYGNTRYADSVKGRFTISRDNAKI
TVYLQMNSLKPEDTAVYYCAAATIGRNAGIDSTTLYDYWGQGTQVTVSS
>SEQ ID NO: 38: 6xHis EPEA tag
>SEQ ID NO: 39: human HBV myristoylated-PreS1 domain
>SEQ ID NO: 40: CDR1 of Nb 87 (Kabat annotation)
>SEQ ID NO: 41: CDR2 of Nb 87 (Kabat annotation)
>SEQ ID NO: 42: CDR3 of Nb 87 (Kabat annotation)
>SEQ ID NO: 43: CDR1 of Nb 91(Kabat annotation)
>SEQ ID NO: 44: CDR2 of Nb 91 (Kabat annotation)
>SEQ ID NO: 45: CDR3 of Nb 91 (Kabat annotation)
>SEQ ID NO: 46: CDR2 of Nb_{NTCP}_87 - S55Q variant
>SEQ ID NO: 47: CDR2 of Nb_{NTCP}_87 - S55E variant
>SEQ ID NO: 48: CDR3 of Nb_{NTCP}_87 - S104G variant
>SEQ ID NO: 49: CDR3 of Nb_{NTCP}_87 - S111R variant
>SEQ ID NO: 50: CDR2 of Nb_{NTCP}_91-N53D variant
>SEQ ID NO: 51: CDR3 of Nb_{NTCP}_91- L103N variant
>SEQ ID NOs: 52-66: sequences disclosed in FIG. 9.
>SEQ ID NO: 67: bivalent Nb87-(GGGGS)x4-Nb87 amino acid sequence
>SEQ ID NO: 68: N-terminal Signal peptide for expression of bivalent Nbs
>SEQ ID NO: 69: human_IGHV3-JH_consensus
>SEQ ID NO: 70-73: humanized variants of Nb87
>SEQ ID NO: 74-75: humanized variants of Nb91
>SEQ ID NO: 76: AntiC2mCherryXL fusion protein with N-terminal signal peptide and C-terminal His-
EPEA tag.

### REFERENCES

1 Doring, B., Lutteke, T., Geyer, J. & Petzinger, E. The SLC10 carrier family: transport functions and molecular structure. Curr Top Membr 70, 105-168 (2012).
2 Yan, H. et al. Sodium taurocholate cotransporting polypeptide is a functional receptor for human hepatitis B and D virus. Elife 1, e00049 (2012).
3 Ni, Y. et al. Hepatitis B and D viruses exploit sodium taurocholate co-transporting polypeptide for species-specific entry into hepatocytes. Gastroenterology 146, 1070-1083 (2014).
4 Lempp, F. A. & Urban, S. Inhibitors of hepatitis B virus attachment and entry. Intervirology 57, 151-157 (2014).
5 Hagenbuch, B. & Dawson, P. The sodium bile salt cotransport family SLC10. Pflugers Arch 447, 566-570 (2004).
6 Claro da Silva, T., Polli, J. E. & Swaan, P. W. The solute carrier family 10 (SLC10): beyond bile acid transport. Mol Aspects Med 34, 252-269 (2013).
7 Hagenbuch, B. & Meier, P. J. Molecular cloning, chromosomal localization, and functional characterization of a human liver Na⁺/bile acid cotransporter. J Clin Invest 93, 1326-1331 (1994).
8 Wong, M. H., Oelkers, P., Craddock, A. L. & Dawson, P. A. Expression cloning and characterization of the hamster ileal sodium-dependent bile acid transporter. J Biol Chem 269, 1340-1347 (1994).
9 Kramer, W. Transporters, Trojan horses and therapeutics: suitability of bile acid and peptide transporters for drug delivery. Biol Chem 392, 77-94 (2011).
10 Tolle-Sander, S., Lentz, K. A., Maeda, D. Y., Coop, A. & Polli, J. E. Increased acyclovir oral bioavailability via a bile acid conjugate. Mol Pharm 1, 40-48 (2004).
11 Kullak-Ublick, G. A. et al. Chlorambucil-taurocholate is transported by bile acid carriers expressed in human hepatocellular carcinomas. Gastroenterology 113, 1295-1305 (1997).
12 Bhat, L., Jandeleit, B., Dias, T. M., Moors, T. L. & Gallop, M. A. Synthesis and biological evaluation of novel steroidal pyrazoles as substrates for bile acid transporters. Bioorg Med Chem Lett 15, 85-87 (2005).
13 Balakrishnan, A. & Polli, J. E. Apical sodium dependent bile acid transporter (ASBT, SLC10A2): a potential prodrug target. Mol Pharm 3, 223-230 (2006).
14 Ho, R. H. et al. Drug and bile acid transporters in rosuvastatin hepatic uptake: function, expression, and pharmacogenetics. Gastroenterology 130, 1793-1806 (2006).
15 Fujino, H., Saito, T., Ogawa, S. & Kojima, J. Transporter-mediated influx and efflux mechanisms of pitavastatin, a new inhibitor of HMG-COA reductase. J Pharm Pharmacol 57, 1305-1311 (2005).
16 Roscam Abbing, R. L. P. et al. Blocking Sodium-Taurocholate Cotransporting Polypeptide Stimulates Biliary Cholesterol and Phospholipid Secretion in Mice. Hepatology 71, 247-258 (2020).
17 Donkers, J. M. et al. Inhibition of Hepatic Bile Acid Uptake by Myrcludex B Promotes Glucagon-Like Peptide-1 Release and Reduces Obesity. Cell Mol Gastroenterol Hepatol 10, 451-466, (2020).
18 Levrero, M., Testoni, B. & Zoulim, F. HBV cure: why, how, when? Curr Opin Virol 18, 135-143, (2016).
19 Zeisel, M. B. et al. Towards an HBV cure: state-of-the-art and unresolved questions—report of the ANRS workshop on HBV cure. Gut 64, 1314-1326, (2015).
20 Engelke, M. et al. Characterization of a hepatitis B and hepatitis delta virus receptor binding site. Hepatology 43, 750-760, (2006).
21 Le Seyec, J., Chouteau, P., Cannie, I., Guguen-Guillouzo, C. & Gripon, P. Infection process of the hepatitis B virus depends on the presence of a defined sequence in the pre-S1 domain. J Virol 73, 2052-2057, (1999).
22 Blanchet, M. & Sureau, C. Infectivity determinants of the hepatitis B virus pre-S domain are confined to the N-terminal 75 amino acid residues. J Virol 81, 5841-5849, (2007).
23 Gripon, P., Cannie, I. & Urban, S. Efficient inhibition of hepatitis B virus infection by acylated peptides derived from the large viral surface protein. J Virol 79, 1613-1622, (2005).
24 Petersen, J. et al. Prevention of hepatitis B virus infection in vivo by entry inhibitors derived from the large envelope protein. Nat Biotechnol 26, 335-341, (2008).
25 Schulze, A., Schieck, A., Ni, Y., Mier, W. & Urban, S. Fine mapping of pre-S sequence requirements for hepatitis B virus large envelope protein-mediated receptor interaction. J Virol 84, 1989-2000, (2010).
26 Glebe, D. et al. Mapping of the hepatitis B virus attachment site by use of infection-inhibiting preS1 lipopeptides and tupaia hepatocytes. Gastroenterology 129, 234-245, (2005).
27 Hu, N. J., Iwata, S., Cameron, A. D. & Drew, D. Crystal structure of a bacterial homologue of the bile acid sodium symporter ASBT. Nature 478, 408-411, (2011).
28 Zhou, X. et al. Structural basis of the alternating-access mechanism in a bile acid transporter. Nature 505, 569-573, (2014).
29 Cirri, E. et al. Consensus designs and thermal stability determinants of a human glutamate transporter. Elife 7 (2018).
30 Uchanski, T. et al. Megabodies expand the nanobody toolkit for protein structure determination by single-particle cryo-EM. Nat Methods 18, 60-68, (2021).
   31 Zahner, D., Eckhardt, U. & Petzinger, E. Transport of taurocholate by mutants of negatively charged amino acids, cysteines, and threonines of the rat liver sodium-dependent taurocholate cotransporting polypeptide Ntcp. Eur J Biochem 270, 1117-1127, (2003).
32 Yan, H. et al. Viral entry of hepatitis B and D viruses and bile salts transportation share common molecular determinants on sodium taurocholate cotransporting polypeptide. J Virol 88, 3273-3284, (2014).
33 Weinman, S. A., Carruth, M. W. & Dawson, P. A. Bile acid uptake via the human apical sodium-bile acid cotransporter is electrogenic. J Biol Chem 273, 34691-34695, (1998).
34 Hagenbuch, B. & Meier, P. J. Sinusoidal (basolateral) bile salt uptake systems of hepatocytes. Semin Liver Dis 16, 129-136, (1996).
35 Ho, R. H., Leake, B. F., Roberts, R. L., Lee, W. & Kim, R. B. Ethnicity-dependent polymorphism in Na+-taurocholate cotransporting polypeptide (SLC10A1) reveals a domain critical for bile acid substrate recognition. J Biol Chem 279, 7213-7222, (2004).
36 Liu, R. et al. Homozygous p.Ser267Phe in SLC10A1 is associated with a new type of hypercholanemia and implications for personalized medicine. Sci Rep 7, 9214, (2017).
37 Pan, W. et al. Genetic polymorphisms in Na⁺-taurocholate co-transporting polypeptide (NTCP) and ileal apical sodium-dependent bile acid transporter (ASBT) and ethnic comparisons of functional variants of NTCP among Asian populations. Xenobiotica 41, 501-510, (2011).
38 Quistgaard, E. M., Low, C., Guettou, F. & Nordlund, P. Understanding transport by the major facilitator superfamily (MFS): structures pave the way. Nat Rev Mol Cell Biol 17, 123-132, (2016).
39 Drew, D. & Boudker, O. Shared Molecular Mechanisms of Membrane Transporters. Annu Rev Biochem 85, 543-572, (2016).
40 Li, H. et al. Clinical and molecular characterization of four patients with NTCP deficiency from two unrelated families harboring the novel SLC10A1 variant c.595A>C (p.Ser199Arg). Mol Med Rep 20, 4915-4924 (2019).
41 Novak, D. A., Ryckman, F. C. & Suchy, F. J. Taurocholate transport by basolateral plasma membrane vesicles isolated from human liver. Hepatology 10, 447-453, (1989).
42 Craddock, A. L. et al. Expression and transport properties of the human ileal and renal sodium-dependent bile acid transporter. Am J Physiol 274, G157-169, (1998).
43 Han, Y. H. et al. Transporter studies with the 3-O-sulfate conjugate of 17alpha-ethinylestradiol: assessment of human kidney drug transporters. Drug Metab Dispos 38, 1064-1071, (2010).
44 Lauger, P. Kinetic properties of ion carriers and channels. J Membr Biol 57, 163-178 (-RETURN) (1980).
45 West, I. C. Ligand conduction and the gated-pore mechanism of transmembrane transport. Biochim Biophys Acta 1331, 213-234, (1997).
46 Ashcroft, F., Gadsby, D. & Miller, C. Introduction. The blurred boundary between channels and transporters. Philos Trans R Soc Lond B Biol Sci 364, 145-147, (2009).
47 Cheng, D., Han, B., Zhang, W. & Wu, W. Clinical effects of NTCP-inhibitor myrcludex B. J Viral Hepat 28, 852-858, (2021).
48 Hehle, V. et al. Potent human broadly neutralizing antibodies to hepatitis B virus from natural controllers. J Exp Med 217, (2020).
49 Corti, D., Benigni, F. & Shouval, D. Viral envelope-specific antibodies in chronic hepatitis B virus infection. Curr Opin Virol 30, 48-57, (2018).
50 Waterhouse, A. M., Procter, J. B., Martin, D. M., Clamp, M. & Barton, G. J. Jalview Version 2—a multiple sequence alignment editor and analysis workbench. Bioinformatics 25, 1189-1191, (2009).
51 Edgar, R. C. MUSCLE: multiple sequence alignment with high accuracy and high throughput. Nucleic Acids Res 32, 1792-1797, (2004).
52 Hattori, M., Hibbs, R. E. & Gouaux, E. A fluorescence-detection size-exclusion chromatography-based thermostability assay for membrane protein precrystallization screening. Structure 20, 1293-1299, (2012).
53 Canul-Tec, J. C. et al. Structure and allosteric inhibition of excitatory amino acid transporter 1. Nature 544, 446-451, (2017).
54 Ritchie, T. K. et al. Chapter 11-Reconstitution of membrane proteins in phospholipid bilayer nanodiscs. Methods Enzymol 464, 211-231, (2009).
55 Pardon, E. et al. A general protocol for the generation of Nanobodies for structural biology. Nat Protoc 9, 674-693, (2014).
56 De Bruyn, T. et al. Confocal imaging with a fluorescent bile acid analogue closely mimicking hepatic taurocholate disposition. J Pharm Sci 103, 1872-1881, (2014).
57 Yamaguchi, K., Murai, T., Yabuuchi, H., Hui, S. P. & Kurosawa, T. Measurement of bile salt export pump transport activities using a fluorescent bile acid derivative. Drug Metab Pharmacokinet 25, 214-219, (2010).
58 Mastronarde, D. N. Automated electron microscope tomography using robust prediction of specimen movements. J Struct Biol 152, 36-51, (2005).
59 Punjani, A., Rubinstein, J. L., Fleet, D. J. & Brubaker, M. A. cryoSPARC: algorithms for rapid unsupervised cryo-EM structure determination. Nat Methods 14, 290-296, (2017).
60 Punjani, A., Zhang, H. & Fleet, D. J. Non-uniform refinement: adaptive regularization improves single-particle cryo-EM reconstruction. Nat Methods 17, 1214-1221, (2020).
61 Pettersen, E. F. et al. UCSF Chimera—a visualization system for exploratory research and analysis. J Comput Chem 25, 1605-1612, (2004).
62 Goddard, T. D. et al. UCSF ChimeraX: Meeting modern challenges in visualization and analysis. Protein Sci 27, 14-25, (2018).
63 Emsley, P. & Cowtan, K. Coot: model-building tools for molecular graphics. Acta Crystallogr D Biol Crystallogr 60, 2126-2132, (2004).
64 Emsley, P., Lohkamp, B., Scott, W. G. & Cowtan, K. Features and development of Coot. Acta Crystallogr D Biol Crystallogr 66, 486-501, (2010).
65 Buchan, D. W. A. & Jones, D. T. The PSIPRED Protein Analysis Workbench: 20 years on. Nucleic Acids Res 47, W402-W407, (2019).
66 Yang, J. et al. The I-TASSER Suite: protein structure and function prediction. Nat Methods 12, 7-8, (2015).
67 Adams, P. D. et al. PHENIX: a comprehensive Python-based system for macromolecular structure solution. Acta Crystallogr D Biol Crystallogr 66, 213-221, (2010).
68 Tian, W., Chen, C., Lei, X., Zhao, J. & Liang, J. CASTp 3.0: computed atlas of surface topography of proteins. Nucleic Acids Res 46, W363-W367, (2018).
69 Pravda, L. et al. MOLEonline: a web-based tool for analyzing channels, tunnels and pores (2018 update). Nucleic Acids Res 46, W368-W373, (2018).
70 Krissinel, E. & Henrick, K. Inference of macromolecular assemblies from crystalline state. J Mol Biol 372, 774-797, (2007).
71 Glaser, F. et al. ConSurf: identification of functional regions in proteins by surface-mapping of phylogenetic information. Bioinformatics 19, 163-164, (2003).
72 Salhab, A., Amer, J., Lu, Y. & Safadi, R. Sodium(+)/taurocholate cotransporting polypeptide as target therapy for liver fibrosis. Gut, (2021).
73 Eller, C. et al. The functional role of sodium taurocholate cotransporting polypeptide NTCP in the life cycle of hepatitis B, C and D viruses. Cell Mol Life Sci 75, 3895-3905, (2018).
74 Gracia-Sancho, J. & Dufour, J. F. NTCP: a pharmacological target for multiple liver conditions. Gut, (2021).
75 Donkers, J. M., Appelman, M. D. & van de Graaf, S. F. J. Mechanistic insights into the inhibition of NTCP by myrcludex B. JHEP Rep 1, 278-285, (2019).
76 Zhang, Q. et al. A safe and efficient hepatocyte-selective carrier system based on myristoylated preS1/21-47 domain of hepatitis B virus. Nanoscale 7, 9298-9310, (2015).
77 Witzigmann, D. et al. Optimization-by-design of hepatotropic lipid nanoparticles targeting the sodium-taurocholate cotransporting polypeptide. Elife 8, (2019).
78 Liu, Q., Somiya, M., lijima, M., Tatematsu, K. & Kuroda, S. A hepatitis B virus-derived human hepatic cell-specific heparin-binding peptide: identification and application to a drug delivery system. Biomater Sci 7, 322-335, (2018).
79 Pardon, E. et al. A general protocol for the generation of Nanobodies for structural biology. Nat Protoc 9, 674-693, (2014).
80 Fanning, G. C., Zoulim, F., Hou, J. & Bertoletti, A. Therapeutic strategies for hepatitis B virus infection: towards a cure. Nat Rev Drug Discov 18, 827-844, (2019).
81 Lucifora, J. & Baumert, T. F. Silencing of the HBV episome through degradation of HBx protein: Towards functional cure? J Hepatol 74, 497-499, (2021).
82 Prescott, N. A., Bram, Y., Schwartz, R. E. & David, Y. Targeting Hepatitis B Virus Covalently Closed Circular DNA and Hepatitis B Virus X Protein: Recent Advances and New Approaches. ACS Infect Dis 5, 1657-1667, (2019).
83 Dong, Z., Ekins, S. & Polli, J. E. Structure-activity relationship for FDA approved drugs as inhibitors of the human sodium taurocholate cotransporting polypeptide (NTCP). Mol Pharm 10, 1008-1019, (2013).
84 Poelstra, K., Prakash, J. & Beljaars, L. Drug targeting to the diseased liver. J Control Release 161, 188-197, (2012).
85 Reddy, L. H. & Couvreur, P. Nanotechnology for therapy and imaging of liver diseases. J Hepatol 55, 1461-1466, (2011).
86 Sawitza, I., Kordes, C., Gotze, S., Herebian, D. & Haussinger, D. Bile acids induce hepatic differentiation of mesenchymal stem cells. Sci Rep 5, 13320 (2015).
87 Takemori T, et al. Establishment of a Monoclonal Antibody against Human NTCP That Blocks Hepatitis B Virus Infection. J Virol. March 9; 96(5): e0168621. (2022).
88 Xu, M. et al. A potent neutralizing antibody with therapeutic potential against all four serotypes of dengue virus. NPJ Vaccines 2, (2017).
89 Schlothauer, T. et al. Novel human IgG1 and IgG4 Fc-engineered antibodies with completely abolished immune effector functions. Protein Eng. Des. Sel. PEDS 29, 457-466 (2016).

1. A composition comprising:
a means for specifically binding the human Na⁺-taurocholate co-transporting polypeptide (NTCP), and
a carrier, diluent, or excipient; and
wherein the means is an allosteric inhibitor of bile salt transport.

2. The composition of claim 1, wherein the means comprises an antibody, an antibody mimetic, a single domain antibody, an immunoglobulin single variable domain (ISVD), or a VHH.

3. The composition of claim 1, wherein the means for specifically binding NTCP comprises an ISVD that stabilizes an inward-facing or open pore NTCP conformational state.

4. A binding agent comprising and antigen binding protein specific for the human Na⁺-taurocholate co-transporting polypeptide (NTCP), wherein the antigen-binding protein comprises an ISVD comprising:
a sequence comprising the complementarity determining regions (CDRs) as presented in any of SEQ ID NOs: 5, 6, 14, or 19-29, wherein the CDRs are annotated according to Kabat, MacCallum, IMGT, AbM, or Chothia, or wherein CDR1 comprises SEQ ID NO: 40, CDR2 comprises SEQ ID NO: 41, 46 or 47, and CDR3 comprises SEQ ID NO: 42, 48, or 49; and/or
a sequence comprising the CDRs as presented in any of SEQ ID NOs: 7-13, 15-18 or 30-37, wherein the CDRs are annotated according to Kabat, MacCallum, IMGT, AbM, or Chothia, or wherein CDR1 comprises SEQ ID NO:43, CDR2 comprises SEQ ID NO:44 or 50, and CDR3 comprises SEQ ID NO: 45 or 51.

5. (canceled)

6. The binding agent of claim 4, wherein the ISVD comprises a sequence selected from the group of sequences of SEQ ID NOs: 5-37, or a functional variant of any one thereof with at least 90% identity over the full length of the ISVD sequence wherein the non-identical amino acids are located in one or more Framework residues.

7. The binding agent of claim 4, wherein the binding agent is a multivalent or multispecific agent, which may comprise an Fc fusion or an antibody.

8. The binding agent of claim 4, wherein the ISVD is labelled, or is conjugated to a functional moiety.

9. The binding agent of claim 8, wherein the functional moiety comprises a therapeutic moiety, a half-life extension, a small-molecule compound, an enzyme, antibody, a genome-editing component, a nucleic acid molecule, a nanoparticle, or a liposome.

10. The binding agent of claim 4, further comprising a therapeutic agent, a carrier, excipient or diluent.

11. (canceled)

12. (canceled)

13. (canceled)

14. (canceled)

15. A screening method to identify a conformation-selective compound of human NTCP, said method comprising the steps of:
a) Combining the means for specifically binding NTCP of claim 1 and a sample comprising NTCP, and
b) Adding a test compound to the sample of a), under suitable conditions to allow binding to NTCP, and
c) measuring that the test compound specifically binds to human NTCP in complex with the binding agent,
and wherein the binding agent is in complex with human NTCP which is present in an inward-facing or an open pore conformational state.

16. The binding agent of claim 4, wherein the ISVD comprises a humanized variant of any one of the Nb87 sequences as presented in any one of SEQ ID NOs: 5, or 25-29, comprising one or more amino acid substitutions at positions corresponding to those in SEQ ID NO: 5 (according to Kabat numbering) selected from Q1, A14, V59, A63, S77, D82a, K83, or Q108.

17. The binding agent of claim 4, wherein the ISVD comprises a humanized variant of any one of Nb91 sequences as presented in any one of SEQ ID NOs: 7, or 30-37, comprising one or more amino acid substitutions at positions corresponding to those in SEQ ID NO:7 (according to Kabat numbering) selected from Q1, E13, A14, I76, K83, or Q108.
